(19) United States
(12) Patent Application Publication
(10) Pub. No.: US 2026/0184725 A1
(43) Pub. Date: Jul. 2, 2026

(54) CYCLIC 2-AMINOPYRIMIDINE COMPOUND AND PHARMACEUTICAL COMPOSITION AND USE THEREOF

(71) Applicants: SHANGHAI INSTITUTE OF ORGANIC CHEMISTRY, CHINESE ACADEMY OF SCIENCES, Shanghai (CN); SHANGHAI INSTITUTE OF MATERIA MEDICA, CHINESE ACADEMY OF SCIENCES, Shanghai (CN)

(72) Inventors: Ke DING, Shanghai (CN); Jian DING, Shanghai (CN); Hao CHEN, Shanghai (CN); Meiyu GENG, Shanghai (CN); Xiaoyun LU, Shanghai (CN); Hua XIE, Shanghai (CN); Zhen WANG, Shanghai (CN); Linjiang TONG, Shanghai (CN)

(73) Assignees: SHANGHAI INSTITUTE OF ORGANIC CHEMISTRY, CHINESE ACADEMY OF SCIENCES, Shanghai (CN); SHANGHAI INSTITUTE OF MATERIA MEDICA, CHINESE ACADEMY OF SCIENCES, Shanghai (CN)

(21) Appl. No.: 19/547,679

(22) Filed: Feb. 24, 2026

Related U.S. Application Data
(63) Continuation of application No. PCT/CN2024/113986, filed on Aug. 22, 2024.

(30) Foreign Application Priority Data
Aug. 25, 2023 (CN) 202311080772.6

(51) Int. Cl. C07D 498/22; A61K 31/529; A61K 31/5377; A61P 35/00

(52) U.S. Cl. CPC C07D 498/22; A61K 31/529; A61K 31/5377; A61P 35/00

(57) ABSTRACT

Provided are a cyclic 2-aminopyrimidine compound having a structure as shown in formula (I), or a pharmaceutically acceptable salt, stereoisomer or prodrug molecule thereof, and a use thereof. The compound can effectively inhibit the activity of EGFR protein kinase drug-resistant mutants (such as EGFR^{T790M} and EGFR^{19del/T790M/C797S}), and can overcome the clinical drug resistance of patients suffering from tumors such as non-small cell lung cancer induced by existing third-generation selective EGFR^{T790M} small molecule inhibitors Osimertinib (AZD9291), Olmutinib (HM6171), Rociletinib (CO-1686) and the like.

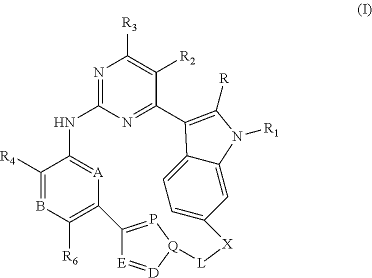

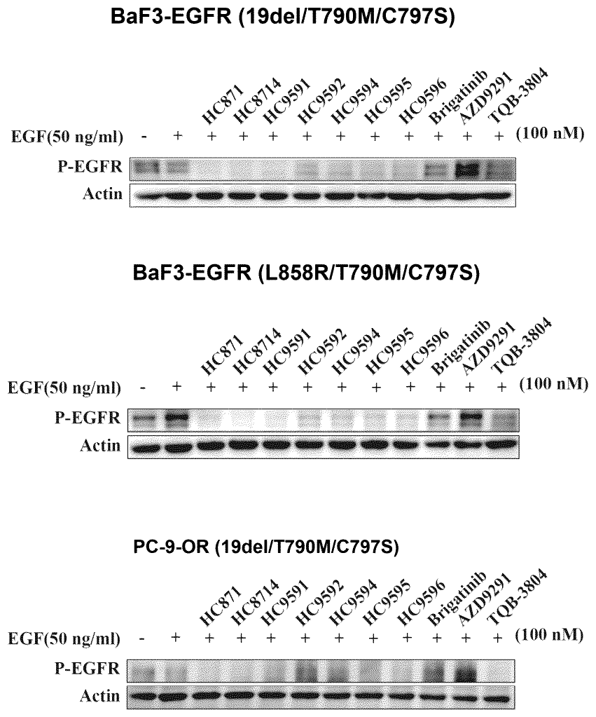

### CROSS-REFERENCE TO RELATED APPLICATION

This application is a continuation of international application of PCT application serial no. PCT/CN2024/113986, filed on Aug. 22, 2024, which claims the priority benefit of China application no. 202311080772.6 filed on Aug. 25, 2023. The entirety of each of the above mentioned patent applications is hereby incorporated by reference herein and made a part of this specification.

### TECHNICAL FIELD

The present disclosure relates to the field of chemical and medical technology, in particular to an application of a class of cyclic 2-aminopyrimidine compound and pharmaceutical composition and use thereof.

### RELATED ART

Continuous proliferation is one of the most important characteristics of tumor cells. Meanwhile, the continuous development and proliferation of tumor cells can damage the structure and function of tissues and organs, leading to the ultimate death of patients due to organ failure. Usually, cell proliferation is strictly regulated by cell proliferation signals, but tumor cells can evade the regulation of proliferation signals through a series of mutations. Therefore, blocking the tumor cell-related mechanism through drugs is an important means of tumor treatment.

Wherein, receptor tyrosine kinases represented by EGFR are key proteins that receive growth factor proliferation signals. Tumor cells can fix the protein in its active conformation through EGFR mutations, enabling it to continuously activate downstream proliferation related signaling pathways without relying on exogenous ligands. In patients with non-small cell lung cancer, approximately 20% have EGFR mutations and exhibit EGFR mutation dependent tumor cell proliferation, with the vast majority being deletion mutations in exon 19 and point mutations in exon 21 (L858R). Therefore, in recent years, various treatment methods targeting EGFR mutations have been applied in clinical practice, such as monoclonal antibodies panitumumab and cetuximab that competitive block of EGFR binding to its ligands, for example EGF and TGF-α, as well as kinase inhibitors gefitinib and osimertinib that mimic ATP structure and block EGFR phosphorylation. The therapy of EGFR inhibition has greatly improved the survival of patients with non-small cell lung cancer. For example, using gefitinib has increased the survival period of patients from less than 10 months in traditional chemotherapy to nearly 30 months. The above evidence indicates that EGFR is a reliable and effective drug target.

The EGFR kinase inhibitors gefitinib and erlotinib, which were launched in 2003 and 2004, respectively, exhibit resistance after a period of use, manifested as a decrease in tumor cell sensitivity to the drugs. One of the main reasons (about 25%) is the occurrence of a second point mutation (T790M) in the kinase region of EGFR, which, on the one hand, hinders drug binding by increasing the steric hindrance of amino acids, reducing the drug's ability to bind to EGFR. On the other hand, it enhances the interaction between the kinase and the substrate ATP, thereby rendering competitive tyrosine kinase inhibitors inactive.

The inhibitor osimertinib, which was launched in 2015, effectively overcomes the resistance problem of T790M point mutation by introducing covalent interaction. It was initially approved as a second-line treatment for patients who developed resistance after treated with gefitinib. Furthermore, research has shown that when used as a first-line medication, Osimertinib can achieve better results than traditional EGFR therapy and effectively target patients with brain metastases. Therefore, covalent EGFR inhibitors represented by Osimertinib are expected to become the preferred medication for patients with non-small cell lung cancer in the foreseeable future.

However, Osimertinib can also develop resistance. Studies have shown that the third point mutation in the kinase region (C797S) can eliminate the ability of covalent binding inhibitors to form covalent bonds, causing the drug to lose its inhibitory activity. This type of mutation has been found in approximately 10% to 20% of patients who are resistant to Osimertinib.

Currently, there are no clinically available inhibitors targeting EGFR^{T790M/C797S} point mutations for the treatment of Osimertinib resistance. Existing literature reports a few inhibitors, but most have significant drawbacks. For example, the allosteric inhibitor EAI045 and its derivatives cannot act on EGFR with exon 19 deletion activation mutation, the ALK inhibitor brigatinib and the quinazoline EGFR inhibitors reported by Park et al. lack sufficient cellular activity, while brigatinib needs to be combined with antibodies to exert in vivo efficacy.

### SUMMARY OF INVENTION

To address the above problems, the present disclosure provides a class of cyclic 2-aminopyrimidine compounds or pharmaceutically acceptable salts or stereoisomers thereof, which can effectively inhibit the activity of the resistant mutants of EGFR protein kinase (such as EGFR^{L858R/T790M}, EGFR^{L858R/T790M/C797S}, EGFR^{19del/T790M/C797S}, etc.) and can be used to treat highly cell proliferative diseases, such as cancer.

The present disclosure includes the following technical solutions.

A Cyclic 2-aminopyrimidine compound with the structure as shown in formula (I), or its pharmaceutically acceptable salts, stereoisomers, or prodrug molecules:

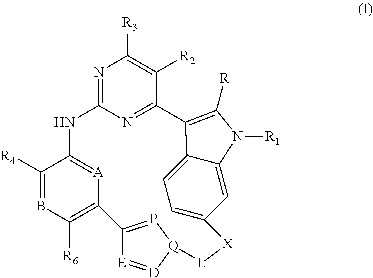

wherein A is selected from: CR₅, N;
B is selected from: CR₇, N;
D. E, Q, and P are independently selected from CR₈ and N, respectively;
X is selected from: —O—, —O(═O)C—, —S—, —S(═O)₂—, —S(═O)—, —C(═O)O—, —C(═O)NH—, —CH₂—, —C═C—, —C≡C—, —NH—, —NH(C═O)—;
R₁ is selected from: H, hydroxyl, C₆˜C₁₀ aryl, 5-10-membered heteroaryl, —C(═O)R₉, —S(═O)₂R₉, —S(═O)R₉, —P(═O)(R₉)₂, R₁₀ substituted or unsubstituted C₁˜C₈ alkyl, R₁₀ substituted or unsubstituted C₃˜C₈ cycloalkyl, R₁₀ substituted or unsubstituted 3-8-membered heterocyclic group; each R₉ is independently selected from: R₁₀ substituted or unsubstituted C₁˜C₈ alkyl, R₁₀ substituted or unsubstituted C₁˜C₈ alkoxy, R₁₀ substituted or unsubstituted C₃˜C₈ cycloalkyl, R₁₀ substituted or unsubstituted C₃˜C₈ cycloalkoxy, amino group, C₁˜C₈ alkylamino, (C₁˜C₈ alkyl)₂ amino; each R₁₀ is independently selected from: H, deuterium, C₁˜C₈ alkyl, C₃˜C₈ cycloalkyl, C₁˜C₈ alkoxy, C₁˜C₈ alkylthio, C₁˜C₈ alkylamino, 3-8-membered heterocyclic group, halogen, hydroxyl, cyano, amino group, —C(═O)R₁₁, —S(═O)₂R₁₁, —S(═O)R₁₁, C₆˜C₁₀ aryl, 5-10-membered heteroaryl; each R₁₁ is independently selected from: C₁˜C₈ alkyl, halogen substituted C₁˜C₈ alkyl, C₁˜C₈ alkoxy, C₂˜C₈ alkenyl;
R₂, R₃, R₄, R₅, R₇, R₈ are independently selected from H, halogen, cyano, nitro, amino group, 5-10-membered heteroaryl, C₆˜C₁₀ aryl, R₁₀ substituted or unsubstituted C₁˜C₈ alkyl, R₁₀ substituted or unsubstituted C₂˜C₈ alkenyl, R₁₀ substituted or unsubstituted C₃˜C₈ cycloalkyl, R₁₀ substituted or unsubstituted C₁˜C₈ alkoxy, R₁₀ substituted or unsubstituted C₃˜C₈ cycloalkoxy, —NR₁₂C(═O)R₁₁, —C(═O)R₁₁, —C(═O)N(R₁₂)₂; each R₁₂ is independently selected from: H, C₁˜C₈ alkyl;
alternatively, R₂, R₃, together with the attached carbon atoms form: a R₁₀ substituted or unsubstituted benzene ring, R₁₀ substituted or unsubstituted C₃˜C₈ cycloalkyl, R₁₀ substituted or unsubstituted 5-8-membered heterocyclic ring, R₁₀ substituted or unsubstituted 5-10-membered heteroaromatic ring;
alternatively, R₄ and R₇, together with the attached carbon atoms form: R₁₀ substituted or unsubstituted benzene ring, R₁₀ substituted or unsubstituted C₃-C₈ cycloalky, R₁₀ substituted or unsubstituted 5-8-membered heterocyclic ring, R₁₀ substituted or unsubstituted 5-10-membered heteroaromatic ring;
R₆ is selected from: —NR₁₃R₁₄, —CR₁₃R₁₄R₁₅, —(CH₂)ₙR₁₃R₁₄R₁₅, —(CH₂)ₙNR₁₃R₁₄; each R₁₃ and each R₁₄ is independently selected from: H, R₁₀ substituted or unsubstituted C₁˜C₈ alkyl, or R₁₃, R₁₄ together with the attached N or C form a substituted or unsubstituted 3 to 12 membered heterocyclic group; R₁₅ is selected from: H, C₁˜C₈ alkyl; N is selected from: 1, 2, 3;
R is selected from H, C₁˜C₈ alkyl, —C(═O)R₁₁, —C(═O)N(R₁₂)₂, or R together with the attached carbon form —C(═O);
L is selected from R₁₆ substituted or unsubstituted C₁˜C₈ alkylene, R₁₆ substituted or unsubstituted C₂˜C₈ alkenediyl;
R₁₆ is selected from: H, deuterium, C₁˜C₈ alkyl, halogen substituted C₁˜C₈ alkyl, C₃˜C₈ cycloalkyl, amino, halogen, hydroxyl, cyano, C₁˜C₈ alkoxy, C₁˜C₈ alkylthio, C₁˜C₈ alkylamino, 3-8-membered heterocyclic group; alternatively, two R₁₆ connected to the same carbon atom or two R₁₆ connected to adjacent carbon atoms to form a C₃˜C₈ cyclic alkyl group.

In some embodiments, the cyclic 2-aminopyrimidine compound has the structure as shown in formula (II) below:

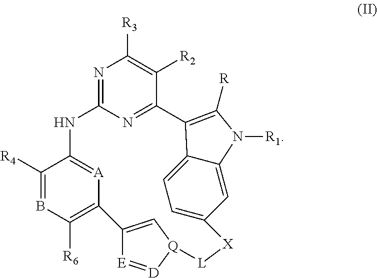

In some embodiments, A is selected from CH, N.

In some embodiments, B is selected from CR₇, N; R₇ is selected from: H, C₅˜C₆ cycloalkyl, C₂˜C₆ alkenyl, and C₁˜C₅ alkyl.

In some embodiments, B is selected from CR₇, N; R₇ is selected from: H, cyclopentyl, methyl vinyl, 2-methylpropenyl, isobutyl, 3,3-dimethylbutenyl, 3,3-dimethylbutyl.

In some embodiments, E is CH; D is CH or N; Q is N.

In some embodiments, E is CH; D is CH or N; Q is N; P is CH.

In some embodiments, E is CH; D is CH or N; Q is N; P is N.

In some embodiments, R₁ is selected from: H, —C(═O)R₉, —S(═O)₂R₉, —S(═O)R₉, —P(═O)(R₉)₂, R₁₀ substituted or unsubstituted C₁˜C₄ alkyl; each R₉ is independently selected from: C₁˜C₄ alkyl, C₁˜C₄ alkoxy, amino, C₁˜C₄ alkylamino, (C₁˜C₄ alkyl) 2-amino, halogen substituted C₁˜C₄ alkyl; R₁₀ in R₁ is selected from H, deuterium, C₁˜C₄ alkyl, C₁˜C₄ alkoxy, halogen, hydroxyl, cyano, and amino.

In some embodiments, R₁ is selected from: H, ethanesulfonyl, methylsulfonyl, propylsulfonyl, methyl, ethyl, propyl, acetyl, trifluoroacetyl, aminoacyl, methylaminoacyl, dimethylaminoacyl, aminosulfonyl, methylaminosulfonyl, dimethylaminosulfonyl, P(═O)(OCH₃)₂, —P(═O)(CH₃)₂.

In some embodiments, R₂ and R₃ are independently selected from: H, halogen, cyano, nitro, amino, R₁₀ substituted or unsubstituted C₁˜C₄ alkyl, R₁₀ substituted or unsubstituted C₂˜C₄ alkenyl, R₁₀ substituted or unsubstituted C₃˜C₆ cycloalkyl, R₁₀ substituted or unsubstituted C₁˜C₄ alkoxy, R₁₀ substituted or unsubstituted C₃˜C₆ cycloalkoxy, —NR₁₂C(═O)R₁₁, —C(═O)R₁₁, —C(═O)N(R₁₂)₂;
each R₁₀ in R₂ and R₃ is independently selected from H, deuterium, C₁˜C₄ alkyl, C₁˜C₄ alkoxy, halogen, hydroxyl, cyano, and amino;
each R₁₁ in R₂ and R₃ is independently selected from: C₁˜C₄ alkyl, halogen substituted C₁˜C₄ alkyl, C₁˜C₄ alkoxy, C₂˜C₄ alkenyl;
each R₁₂ in R₂ and R₃ is independently selected from: H, C₁˜C₄ alkyl;
alternatively, R₂, R₃, together with the attached carbon atoms form R₁₀ substituted or unsubstituted benzene ring, R₁₀ substituted or unsubstituted C₅˜C₆ cycloalkyl, R₁₀ substituted or unsubstituted 5-6-membered heterocyclic ring, or R₁₀ substituted or unsubstituted 5-6 membered heteroaromatic ring.

In some embodiments, R₂ is selected from the group consisting of H, chlorine, bromine, fluorine, iodine, methyl, methoxy, methoxycarbonyl, ethoxycarbonyl, isopropoxycarbonyl, nitro, amino, trifluoromethyl, acetamido, acrylamido, isobutyramido, propionamido, and cyano; R₃ is H;
alternatively, R₂, R₃, together with the attached carbon atoms form: benzene ring, pyrrole ring, thiophene ring, pyrazole ring, pyrazole ring, pyridine ring, furan ring.

In some embodiments, when R₄ is selected from H or methyl, R₂ is not H.

In some embodiments, R₄ is selected from: H, halogen, cyano, nitro, amino, R₁₀ substituted or unsubstituted C₁˜C₄ alkyl, R₁₀ substituted or unsubstituted C₂˜C₄ alkenyl, R₁₀ substituted or unsubstituted C₃˜C₆ cycloalkyl, R₁₀ substituted or unsubstituted C₁˜C₄ alkoxy, R₁₀ substituted or unsubstituted C₃˜C₆ cycloalkoxy, —NR₁₂C(═O)R₁₁, —C(═O)R₁₁, —C(═O)N(R₁₂)₂;
each R₁₀ in R₄ is independently selected from: H, deuterium, C₁˜C₄ alkyl, C₁˜C₄ alkoxy, halogen, hydroxyl, cyano, and amino;
each R₁₁ in R₄ is independently selected from: C₁˜C₄ alkyl, halogen substituted C₁˜C₄ alkyl, C₁˜C₄ alkoxy, C₂˜C₄ alkenyl;
each R₁₂ in R₄ is independently selected from: H and C₁˜C₄ alkyl.

In some embodiments, R₄ is selected from: H, methyl, ethyl, isopropyl, methoxy, ethoxy, isopropoxy, n-propoxy, isobutyloxy, deuterated methoxy, trifluoromethoxy, cyclopropoxy, trifluoromethyl, and cyclopropyl.

In some embodiments, R₄ is selected from H, methyl; R₂ is selected from: chlorine, bromine, fluorine, iodine, methyl, methoxy, methoxycarbonyl, ethoxycarbonyl, isopropoxycarbonyl, nitro, amino, trifluoromethyl, acetamido-, acrylamido-, isobutyramido-, propionamido-; R₃ is H.

In some embodiments, R₄ is selected from: methoxy, ethoxy, isopropoxy, n-propoxy, isobutyloxy, deuterated methoxy, trifluoromethoxy, cyclopropoxy, trifluoromethyl, cyclopropyl, isopropyl, and ethyl; R₂ is selected from: H, chlorine, bromine, fluorine, iodine, methyl, methoxy, methoxycarbonyl, ethoxycarbonyl, isopropoxycarbonyl, nitro, amino, trifluoromethyl, acetamido-, acrylamido-, isobutyramido-, propionamido-, cyano; R₃ is H.

In some embodiments, R₁ is selected from: ethanesulfonyl, methylsulfonyl, and propanesulfonyl; one of R₂ and R₄ is H, and the other is not H; R₃ is H.

In some embodiments, R₆ is selected from: —NR₁₃R₁₄, —CR₁₃R₁₄R₁₅, —(CH₂)ₙR₁₃R₁₅, —(CH₂)ₙNR₁₃R₁₄; each R₁₃ and R₁₄ is independently selected from H, C₁˜C₄ alkyl, dimethylamino substituted C₁˜C₄ alkyl, or R₁₃, R₁₄ together with the attached N or C form R₁₇ substituted or unsubstituted 3-12 membered heterocyclic groups; R₁₅ is selected from: H, C₁˜C₄ alkyl;
each R₁₇ is independently selected from: H, deuterium, C₁˜C₈ alkyl, hydroxyl substituted C₁˜C₈ alkyl, C₃˜C₈ cycloalkyl, C₁˜C₈ alkoxy, C₁˜C₈ alkylthio, C₁˜C₈ alkylamino, halogen, hydroxyl, cyano, amino, R₁₈ substituted or unsubstituted 3-8-membered heterocyclic group, —C(═O)R₁₁, —S(═O)₂R₁₁, —S(═O)R₁₁, or R₁₇ together with the attached carbon atom form C(═O); R₁₈ is selected from: H, C₁˜C₈ alkyl.

In some embodiments, each R₁₇ is independently selected from: H, deuterium, C₁˜C₄ alkyl, hydroxyl substituted C₁˜C₄ alkyl, C₅˜C₆ cycloalkyl, C₁˜C₄ alkoxy, C₁˜C₄ alkylthio, dimethylamino, halogen, hydroxyl, cyano, amino, R₁₈ substituted or unsubstituted 3-8-membered heterocyclic group, —C(═O)R₁₁, —S(═O)₂R₁₁, —S(═O)R₁₁, or R₁₇ forms C(═O) together with the attached carbon atom; R₁₈ is selected from: H, C₁˜C₄ alkyl;
each R₁₁ in R₁₇ is independently selected from: C₁˜C₄ alkyl, C₁˜C₄ alkoxy.

In some embodiments, R₆ is selected from:

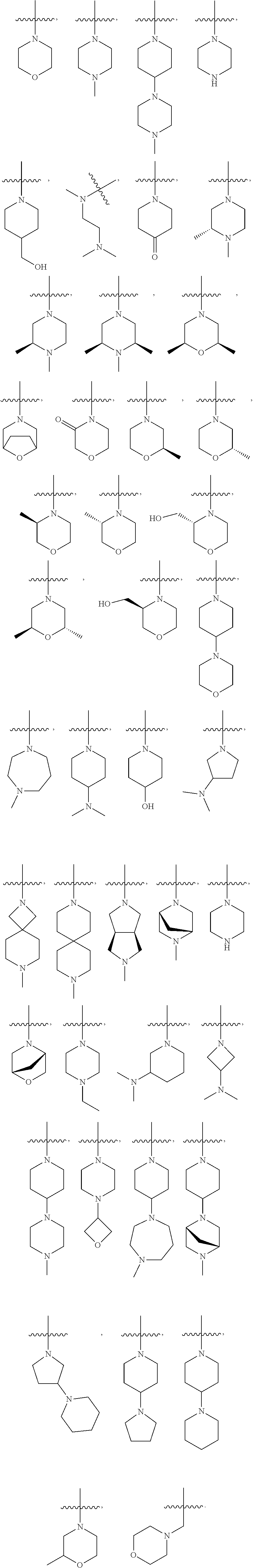

In some embodiments, R is selected from H, methoxycarbonyl, isopropoxycarbonyl, aminoformyl, methyl, ethoxycarbonyl, or R together with the attached carbon atom forms C(═O).

In some embodiments, L is selected from R₁₆ substituted or unsubstituted C₂˜C₆ alkylene, R₁₆ substituted or unsubstituted C₂˜C₆ alkenediyl;

R₁₆ is selected from: H, deuterium, C₁˜C₄ alkyl, halogen substituted C₁˜C₄ alkyl, amino, halogen, hydroxyl, cyano, C₁˜C₄ alkoxy, C₁˜C₄ alkylthio, C₁˜C₄ alkylamino; alternatively, a cyclopropyl group formed by two R₁₆ connected to the same carbon atom.

In some embodiments, L is selected from: —(CH₂)₂—, —(CH₂)₃—, —(CH₂)₄—,

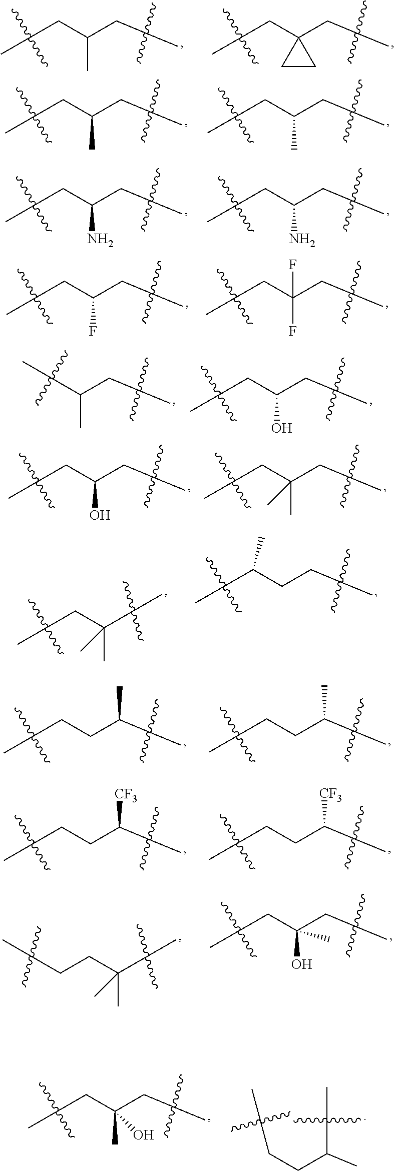

The present disclosure also provides an application of the cyclic 2-aminopyrimidine compound, its pharmaceutically acceptable salt, stereoisomer, or its prodrug molecule, including the following technical solutions.

Use of the cyclic 2-aminopyrimidine compound or its pharmaceutically acceptable salts or stereoisomers or prodrug molecules in the preparation of EGFR inhibitors.

Use of the cyclic 2-aminopyrimidine compound or its pharmaceutically acceptable salts or stereoisomers or prodrug molecules in the preparation of mutant EGFR inhibitors, wherein the mutant EGFR is EGFR^{T790M}, EGFRT^{790M/C797S}, EGFR^{L858R/T790M}, EGFR^{19del/T790M/C797S}, EGFR^{L858R/C797S}, EGFR^{L858R/T790M/C797S}.

Use of the cyclic 2-aminopyrimidine compound or its pharmaceutically acceptable salts or stereoisomers or prodrug molecules in the preparation of drugs for preventing and/or treating tumors.

In some embodiments, the tumor is a malignant tumor with EGFR gene mutation.

In some embodiments, the tumor is a malignant tumor with EGFR^{19del/T790M/C797S} mutation, EGFR^{T790M} mutation, EGFR^{T790M/C797S} mutation, EGFR^{L858R/T790M} mutation, EGFR^{L858R/C797S} mutation and/or EGFR^{L858R/T790M/C797S} mutation.

In some embodiments, the tumor is: non-small cell lung cancer, small cell lung cancer, lung adenocarcinoma, lung squamous cell cancer, pancreatic cancer, breast cancer, prostate cancer, liver cancer, skin cancer, epithelial cell cancer, gastrointestinal stromal tumor, leukemia, histiocytic lymphoma, nasopharyngeal cancer, head and neck tumor, colon cancer, rectal cancer or glioma.

The present disclosure also provides a pharmaceutical composition for preventing and/or treating tumors, comprising the following technical solutions.

A pharmaceutical composition for preventing and/or treating tumors, being prepared from an active ingredient and a pharmaceutically acceptable excipient, wherein the active ingredient comprises a cyclic 2-aminopyrimidine compound or its pharmaceutically acceptable salt or stereoisomer or a prodrug molecule as described in the present invention.

The present disclosure also provides a method of preventing and/or treating tumors, wherein the method comprises: administering to a patient a safe and effective amount of the cyclic 2-aminopyrimidine compound or its pharmaceutically acceptable salts or stereoisomers or prodrug molecules as described in the present invention; and/or,
administering a safe and effective amount of the pharmaceutical composition.

In some embodiments, the tumors are non-small cell lung cancer, small cell lung cancer, lung adenocarcinoma, lung squamous cell cancer, pancreatic cancer, breast cancer, prostate cancer, liver cancer, skin cancer, epithelial cell cancer, gastrointestinal stromal tumor, leukemia, histiocytic lymphoma, nasopharyngeal cancer, head and neck tumor, colon cancer, rectal cancer or glioma.

The present disclosure also provides a method for selectively inhibiting mutant EGFR kinase activity, comprising: administering to a patient a safe and effective amount of cyclic 2-aminopyrimidine compounds or their pharmaceutically acceptable salts or stereoisomers or prodrug molecules as described in the present invention; and/or, administering a safe and effective amount of a pharmaceutical composition thereof.

In some embodiments, the mutant EGFR kinase is EGFR^{T790M}, EGFRT^{790M/C797S}, EGFR^{19del/T790M/C797S}, EGFR^{L858R/T790M}, EGFR^{L858R/C797S}, or EGFR^{L858R/T790M/C797S}.

Compared with the existing technology, the present disclosure has the following beneficial effects:
(1) The cyclic 2-aminopyrimidine compound or its pharmaceutically acceptable salts or stereoisomers or prodrug molecules of the present disclosure can inhibit the growth of various tumor cells by inhibiting EGFR family proteases. Compared to wild-type cancer cells, the compounds of the present disclosure exhibit higher selectivity on mutant cancer cells. The compounds of the present disclosure are particularly effective in inhibiting the activity of EGFR protein kinase resistant mutants (such as EGFR^{T790M}, EGFR^{T790M/C797S}, EGFR^{19del}, EGFR^{858R/T790M/C797S}, and EGFR^{19del/T790M/C797S}, etc.), selectively acting on EGFR^{858R/T790M}, EGFR^{19del}, and EGFR^{19del/T790M/C797S} lung cancer cells, and overcoming clinical resistance induced by existing third-generation selective EGFR^{T790M} small molecule inhibitors Osimertinib (AZD9291), Olmutinib (HM6171), Rociletinib (CO-1686), etc, in patients with non-small cell lung cancer and other tumors.
(2) The compound of the present disclosure can be used to prepare anti-tumor drugs and overcome resistance induced by existing drugs such as gefitinib, erlotinib, especially Osimertinib (AZD9291). It is a novel protein kinase inhibitor that can overcome resistance to existing EGFR tyrosine kinase inhibitors and has selective and good pharmacokinetic properties, and can be used to treat hyperproliferative diseases such as tumors in humans and other mammals.

### BRIEF DESCRIPTION OF DRAWINGS

FIGURE shows the inhibition of EGFR phosphorylation levels by the cyclic 2-aminopyrimidine compounds in different EGFR mutant cells.

### DESCRIPTION OF EMBODIMENTS

In the compound of the present disclosure, when any variable (e.g. R₉, etc.) occurs more than once in any component, its definition at each occurrence is independent from the definition at each other occurrence. Similarly, combinations of substituents and variables are permissible only if the compound with such combinations are stabilized. A line from a substituent to a ring system indicates that the indicated bond may be attached to any substitutable ring atom. If the ring system is polycyclic, it means that such bonds are only attached to any suitable carbon atoms adjacent to the ring. It is understood that an ordinary skilled in the art can select substituents and substitution patterns of the compound of the present disclosure to provide compounds that are chemically stable and can be readily synthesized from the available starting materials by the methods described below. If a substituent itself is substituted by more than one group, it should be understood that these groups may be on the same carbon atom or on different carbon atoms, so long as the structure is stabilized.

The phrase “R_{f} substituted”, “R substituted” used in the present disclosure are considered equivalent to the phrase “substituted by at least one substituent”, and in this case, preferred embodiments will have 1 to 4 identical or different substituents from each other

The term “alkyl” in the present disclosure is meant to include branched and straight chain saturated aliphatic hydrocarbon groups having the specified number of carbon atoms. For example, the definition of “C₁-C₈” in “C₁-C₈ alkyl” includes groups having 1, 2, 3, 4, 5 or 6 carbon atoms arranged in a straight or branched chain. For example, “C₁-C₄ alkyl” specifically includes methyl, ethyl, n-propyl, isopropyl, n-butyl, tert-butyl, isobutyl, pentyl, hexyl.

The term “cycloalkyl” refers to a monocyclic saturated fatty hydrocarbon group with a specific number of carbon atoms. For example, “cycloalkyl” includes cyclopropyl, cyclobutyl, cyclopentyl, or cyclohexyl groups.

The term “alkoxy” refers to a group with an —O-alkyl structure, such as —OCH₃, —OCH₂CH₃, —OCH₂CH₂CH₃, —O—CH₂CH(CH₃)₂, —OCH₂CH₂CH₂CH₃, —O—CH(CH₃)₂, etc.

The term “heterocycle” refers to a saturated or partially unsaturated monocyclic or polycyclic ring substituent (including monocyclic, spirocyclic, fused cyclic, bridged cyclic, etc.), in which one or more ring atoms are heteroatoms selected from N, O, or S(O)m (where m is an integer from 0 to 2), and the remaining ring atoms are carbon.

The term “heteroaromatic group” refers to an aromatic ring containing 1, 2, or 3 heteroatoms selected from O, N, or S. Heteroaromatic groups within the scope of the present disclosure include but are not limited to: quinazoline, quinoline, pyrazole, pyrrole, thiophene, furan, pyridine, pyrimidine, pyrazine, triazole, imidazole, oxazole, isoxazole, pyridazine, etc.

The term ‘halogen’ or ‘halogen’ refers to chlorine, fluorine, bromine, and iodine.

The present disclosure includes free forms of compound of Formula I, as well as pharmaceutically acceptable salts and stereoisomers thereof. The stereoisomers in the present disclosure (depending on their structure) are enantiomers, diastereomers, syn/anti isomers, cis/trans isomers, epimers, and (E)/(Z) isomers. The compound of formula (I) can be used in the context of the present invention in the form of pure stereoisomers or any mixture of stereoisomers, in which latter case a racemate is preferred.

The term “free form” refers to the amine compounds in non-salt form. Some specific exemplary compounds in the present disclosure are protonation salts of amine compounds. The pharmaceutically acceptable salts include not only exemplary salts of the particular compounds described herein, but also typical pharmaceutically acceptable salts of free form of all compounds of Formula I. The free forms of specific salts of the compounds can be isolated using techniques known in the art. For example, the free form can be regenerated by treating the salt with an appropriate dilute aqueous base, such as dilute aqueous NaOH, dilute aqueous potassium carbonate, dilute aqueous ammonia, and dilute aqueous sodium bicarbonate. The free forms differ somewhat from their respective salt forms in certain physical properties such as solubility in polar solvents, but for the purposes of the disclosure, such salts of acid or base are otherwise pharmaceutically equivalent to their respective free forms.

The pharmaceutically acceptable salts of the present disclosure can be synthesized from the compounds containing a basic or acidic moiety in the present disclosure by conventional chemical methods. Generally, salts of basic compounds can be prepared by ion exchanged chromatography or by reacting the free base with a stoichiometric or excess amount of inorganic or organic acid in the desired salt form in a suitable solvent or combination of solvents. Similarly, salts of acidic compounds can be formed by reaction with a suitable inorganic or organic base.

Therefore, the pharmaceutically acceptable salts of the compounds in the present disclosure include conventional non-toxic salts of the compounds in the present disclosure formed by reacting a basic compound of the present disclosure with an inorganic or organic acid. For example, conventional non-toxic salts include those derived from inorganic acids such as hydrochloric acid, hydrobromic acid, sulfuric acid, sulfamic acid, phosphoric acid, nitric acid, etc. They also include salts derived from organic acids such as acetic acid, propionic acid, succinic acid, glycolic acid, hard Fatty acid, lactic acid, malic acid, tartaric acid, citric acid, ascorbic acid, pamoic acid, maleic acid, hydroxymaleic acid, phenylacetic acid, glutamic acid, benzoic acid, salicylic acid, p-aminobenzenesulfonic acid, 2-acetoxy-benzoic acid, fumaric acid, toluenesulfonic acid, methanesulfonic acid, ethanedisulfonic acid, oxalic acid, isethionic acid, and trifluoroacetic acid, etc.

If the compounds of the present disclosure are acidic, the appropriate “pharmaceutically acceptable salts” refer to the salts prepared from pharmaceutically acceptable non-toxic bases including inorganic bases and organic bases. The salts derived from inorganic bases include aluminum, ammonium, calcium, copper, iron, ferrous, lithium, magnesium, manganese, manganous, potassium, sodium, zinc, etc. Particularly preferred, ammonium salts, calcium salts, magnesium salts, potassium salts, and sodium salts. The salts derived from pharmaceutically acceptable organic non-toxic bases include salts of primary, secondary and tertiary amines. Substituted amines include naturally occurring substituted amines, cyclic amines and basic ion exchange resins such as Amino acid, betaine, caffeine, choline, N,N′-dibenzylethylenediamine, diethylamine, 2-diethylaminoethanol, 2-dimethylaminoethanol, aminoethanol, ethanolamine, ethyl Diamine, N-ethylmorpholine, N-ethylpiperidine, Glucosamine, Glucosamine, Histidine, Hydroxocobalamin, Isopropylamine, Lysine, Methylglucamine, Morpholine, Piperazine, Piperidine, quack, polyamine resin, procaine, purine, theobromine, triethylamine, trimethylamine, tripropylamine, tromethamine, etc.

The preparation of the pharmaceutically acceptable salts described above and other typical pharmaceutically acceptable salts was described in more detail by Berg et al., in “Pharmaceutical Salts,” J. Pharm. Sci. 1977:66:1-19.

Since under physiological conditions, the deprotonated acidic moieties (such as carboxyl groups) in compounds can be anions, which carry electric charges and can be neutralized by internally cationic protonated or alkylated basic moieties (such as tetravalent nitrogen atoms), so it should be noted that the compounds of the present disclosure are potential internal salts or zwitterions.

In one embodiment, the present disclosure provides a method for treating transitional proliferative diseases or symptoms such as human or other mammalian tumors using compounds with the structure shown in formula (I) and its pharmaceutically acceptable salts.

In one embodiment, the compound and its pharmaceutically acceptable salt can be used to treat or control non-small cell lung cancer, small cell lung cancer, lung adenocarcinoma, lung squamous cell cancer, pancreatic cancer, breast cancer, prostate cancer, liver cancer, skin cancer, epithelial cell cancer, gastrointestinal stromal tumor, leukemia, histiocytic lymphoma, nasopharyngeal cancer, head and neck tumors, colon cancer, rectal cancer or glioma, etc.

The present disclosure also provides a pharmaceutical composition comprising active ingredients within a safe and effective dosage range, as well as pharmaceutically acceptable excipients.

The “active ingredient” described in the present disclosure includes the compound of formula I described in the present disclosure.

The “active ingredient” and pharmaceutical combination described in the present disclosure can be used as mutant EGFR inhibitors. In another preferred embodiment, for the preparation of drugs for the prevention and/or treatment of tumors.

“Safe and effective dosage” refers to the amount of the active ingredients that are sufficient to significantly improve the condition without causing serious side effects.

“Pharmaceutically acceptable carrier or excipient” refers to one or more compatible solid or liquid fillers or gel substances, which are suitable for human use, and must have sufficient purity and sufficiently low toxicity.

“Compatibility” here refers to the ability of each component of the composition to be mixed with the active ingredients of the present disclosure intermingled between each other without significantly reducing the efficacy of the active ingredients.

Examples of pharmaceutically acceptable carriers or excipients include cellulose and its derivatives (such as sodium carboxymethyl cellulose, sodium ethylcellulose, cellulose acetate, etc.), gelatin, talc, solid lubricants (such as stearic acid, magnesium stearate), calcium sulfate, vegetable oil (such as soybean oil, sesame oil, peanut oil, olive oil, etc.), polyols (such as propylene glycol, glycerin, mannitol, sorbitol, etc.), emulsifier (such as Tween®), wetting agent (such as sodium dodecyl sulfate), colorant, flavoring agent, stabilizer, antioxidant, preservative, pyrogen-free water, etc.

There are no special restrictions on application methods of the active ingredients or drug composition of the present disclosure, and typical administration methods include (but not limited to) oral, intratumoral, rectal, parenteral (intravenous, intramuscular or subcutaneous), etc.

The solid dosage forms used for oral administration include capsules, tablets, pills, powders and granules.

In these solid dosage forms, the active ingredient is mixed with at least one conventional inert excipient (or carrier), such as sodium citrate or dicalcium phosphate, or with the mixture of following ingredients:
(a) fillers or compatibilizers, such as starch, lactose, sucrose, glucose, mannitol, and silicic acid;
(b) adhesives, such as hydroxymethyl cellulose, alginate, gelatin, polyvinylpyrrolidone, sucrose, and arabic gum;
(c) humectants, such as glycerin;
(d) disintegrating agents, such as agar, calcium carbonate, potato starch or cassava starch, algic acid, some composite silicates, and sodium carbonate;
(e) slow solvent, such as paraffin;
(f) absorption accelerators, such as quaternary amine compounds;
(g) wetting agents, such as cetyl alcohol and glyceryl monostearate;
(h) adsorbents, such as kaolin; and
(i) lubricants, such as talc, calcium stearate, magnesium stearate, solid polyethylene glycol, sodium dodecyl sulfate, or their mixtures. In the case of capsules, tablets and pills, the dosage form may also include buffers.

The solid dosage form can also be prepared with coating and shell materials, such as casings and other materials known in the art. They may comprise an opaque agent. Furthermore, the active ingredients from such compositions may be released in certain part of the digestive tract in a delayed manner. Examples of embedding components that can be used are polymers and waxes.

Liquid dosage forms for oral administration include pharmaceutically acceptable lotion, solutions, suspensions, syrups or tinctures. In addition to the active ingredients, the liquid dosage form may include inert diluents commonly used in the art, such as water or other solvents, solubilizers, emulsifiers (such as ethanol, isopropanol, ethyl carbonate, ethyl acetate, propylene glycol, 1,3-butanediol, dimethylformamide), and oil (especially cottonseed oil, peanut oil, corn germ oil, olive oil, castor oil and sesame oil or mixtures of these substances). In addition to these inert diluents, the composition may also include auxiliary agents, such as wetting agents, emulsifiers and suspending agents, sweeteners, flavoring agents and spices.

In addition to the active ingredients, the suspension may contain suspension agents, such as ethoxylated isooctadecanol, polyoxyethylene sorbitol and dehydrated sorbitol ester, microcrystalline cellulose, aluminum methoxide and agar, or mixtures of these substances.

Compositions for parenteral injection may include physiologically acceptable sterile aqueous or anhydrous solutions, dispersions, suspensions or emulsions, and sterile powders for re-dissolution into sterile injectable solutions or dispersions. Suitable aqueous and non-aqueous carriers, diluents, solvents or excipients include water, ethanol, polyols, and their suitable mixtures.

The compounds of the present disclosure can be administered alone or in combination with other therapeutic agents, such as hypoglycemic agents.

When using as a pharmaceutical composition, a safe and effective amount of the compound of the present disclosure is administered to a mammal (such as a human) in need of treatment, wherein the dosage is considered to be an effective drug delivery dose in pharmacology. Of course, the specific dosage should also take into account factors such as the route of administration and the patient's health status, these are all within the scope of a skilled physician's expertise.

Combination therapy; the compounds of formula (I) can be used in combination with other drugs known to treat or ameliorate similar conditions. In the case of combined administration, the administration method and dosage of the original drug remain unchanged, while the compound of Formula (I) is administered simultaneously or subsequently. When the compound of Formula (I) is administered concomitantly with one or more other drugs, it is preferred to use a pharmaceutical composition containing one or more known drugs and the compound of Formula (I). Drug combination also includes administration of the compound of Formula (I) with one or more other drugs in overlapping time periods. When the compound of Formula (I) is used in combination with one or more other drugs, the compound of Formula (I) or the drugs may be administered at lower doses than that when they are administered alone.

The drugs or active ingredients that can be used in combination with compounds of formula (I) include but are not limited to:

Estrogen receptor modulators, androgen receptor modulators, retinal like receptor modulators, cytotoxic/cell inhibitors, anti proliferative agents, protein transferase inhibitors, HMG CoA reductase inhibitors, HIV protein kinase inhibitors, reverse transcriptase inhibitors, angiogenesis inhibitors, cell proliferation and survival signal inhibitors, drugs that interfere with cell cycle checkpoints, and apoptosis inducers, cytotoxic drugs, tyrosine protein inhibitors EGFR inhibitors, VEGFR inhibitors, serine/threonine protein inhibitors, Bcr Abl inhibitors, c-Kit inhibitors, Met inhibitors, Raf inhibitors, MEK inhibitors, MMP inhibitors, topoisomerase inhibitors, histidine deacetylase inhibitors, proteasome inhibitors, CDK inhibitors, Bcl-2 family protein inhibitors, MDM2 family protein inhibitors, IAP family protein inhibitors, STAT family protein inhibitors, PI3K inhibitors, AKT inhibitors, integrin blockers, interferon-α, interleukin-12, COX-2 inhibitors, p53, p53 activators, VEGF antibodies, EGF antibodies, etc.

The present disclosure will be further explained in conjunction with specific embodiments. It should be understood that these embodiments are only used to illustrate the present disclosure and not to limit the scope of the present disclosure. The experimental methods without specific conditions specified in the following embodiments are usually carried out under conventional conditions or conditions recommended by the manufacturer. Unless otherwise specified, percentages and portions are calculated by weight.

Unless otherwise defined, all professional and scientific terms used in the text have the same meanings as those familiar to technical personnel in this field. In addition, any method and material similar or equivalent to the described content can be applied to the method of the present disclosure. The preferred implementation methods and materials described in the article are for demonstration purposes only.

The following are specific implementation embodiments.

### Embodiment 1

### Preparation of (Z)-2⁵-Chloro-1¹-(ethylsulfonyl)-4⁴-morpholino-1¹H,5¹H-9-oxa-3-aza-1(3,6)-indola-2 (4,2)-pyrimidina-5(4,1)-pyrazola-4(1,3)-benzenacyclononaphane9-oxa-3-azaHC871)

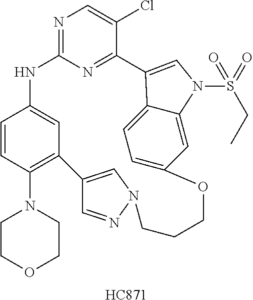 [SMILES: CCS(=O)(=O)n1cc2c3ccc(cc31)OCCCn1cc(cn1)-c1cc(ccc1N1CCOCC1)Nc1ncc(Cl)c-2n1 ; "HC871"]

### Step 1. Preparation of 3-(2,5-dichloropyrimidin-4-yl)-6-methoxy-1H-indole (3)

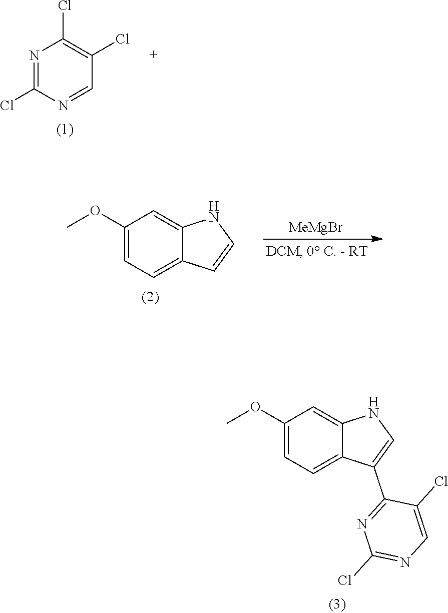 [SMILES: "MeMgBr" ; Clc1ncc(Cl)c(Cl)n1 ; COc1ccc2cc[nH]c2c1 ; COc1ccc2c(-c3nc(Cl)ncc3Cl)c[nH]c2c1]

6-methoxy-1H-indole (1 g, 6.79 mmol) was dissolved in 25 mL of dry dichloromethane, and cooled to 0° C. in an ice bath under the argon protection. A 3 mol/L solution of methyl magnesium bromide in diethyl ether (3.4 mL, 10.18 mmol) was added dropwise to the solution, followed with dissolving 2,4,5-trichloropyrimidine (1.25 g, 6.79 mmol) in another 25 mL of dry dichloromethane, and the latter was added dropwise to the reaction system. Then, the reaction was moved to room temperature and continued to proceed for 3 hours. The reaction progress was monitored by TLC (developing agent: petroleum ether:ethyl acetate=5:1, v/v). After the reaction was completed, the ice bath was restored and water (50 mL) was added dropwise for quenching to precipitate a yellow solid. The obtained mixture was filtered and the filter cake was washed twice with ethyl acetate until it turned light yellow. The filter cake was collected and dried to obtain 1.1 g of light yellow solid, i.e. the compound in title, with a yield of 55%.

¹H NMR (400 MHz, DMSO-d₆) δ 12.09 (s, 1H), 8.69 (s, 1H), 8.62 (s, 1H), 8.39 (d, 1H), 7.03 (s, 1H), 6.91 (d, J=8.7 Hz, 1H), 3.81 (s, 3H).

### Step 2. Preparation of 3-(2,5-dichloropyrimidin-4-yl)-1-(ethylsulfonyl)-6-methoxy-1H-indole (4)

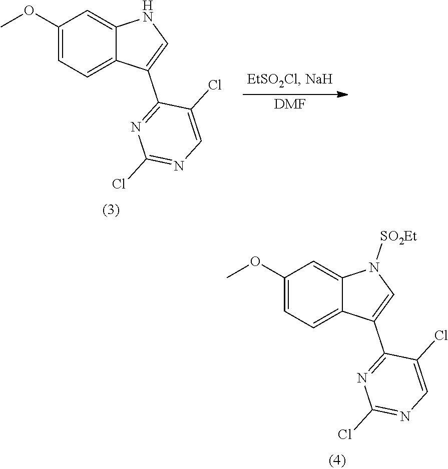

3-(2,5-dichloropyrimidin-4-yl)-6-methoxy-1H-indole (1.1 g, 3.74 mmol) was dissolved in 10 mL DMF and the reaction was cooled in an ice bath to 0° C. 60% by mass of sodium hydride (0.22 g, 5.61 mmol) dispersed in mineral oil was added in portions. The mix was reacted in an ice bath for 10 minutes, and ethyl sulfonyl chloride (0.42 mL, 4.49 mmol) was slowly added. Then, the reaction was moved to room temperature and continued to proceed for 1 hour. The reaction progress was monitored by TLC (developing agent: petroleum ether:ethyl acetate=5:1, v/v). After the reaction was completed, water (50 mL) was added dropwise for quenching to precipitate a white solid. The obtained mixture was filtered and the filter cake was washed twice with methanol. The filter cake was collected and dried to obtain 2 g of white solid with a yield of 83%, i.e. the compound in title.

¹H NMR (400 MHz, DMSO-d₆) δ 8.95 (s, 1H), 8.50 (s, 1H), 8.32 (d, J=8.9 Hz, 1H), 7.41 (d, J=2.2 Hz, 1H), 7.13 (dd, J=8.9, 2.3 Hz, 1H), 3.87 (s, 3H), 3.87-3.82 (m, 2H), 1.14 (t, J=7.3 Hz, 3H).

### Step 3. Preparation of 3-(2,5-dichloropyrimidin-4-yl)-1-(ethylsulfonyl)-1H-indole-6-ol (5)

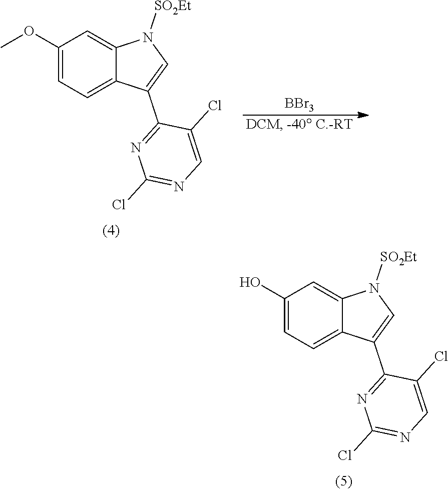

3-(2,5-dichloropyrimidin-4-yl)-1-(ethylsulfonyl)-6-methoxy-1H-indole (0.2 g, 0.5 mmol) was dissolved in 3 mL of dry dichloromethane and the reaction mixture was cooled to −40° C. 1 mol/L solution of boron tribromide (3 mL, 3.11 mmol) was added dropwise into dichloromethane. After the addition completed, the reaction was moved to room temperature and continued for 5 hours. During the progress, a sample was diluted with ethyl acetate and saturated sodium bicarbonate solution. The upper layer of the mixture was taken, and the reaction progress was monitored by TLC (developing agent: petroleum ether:ethyl acetate=5:1, v/v). After the reaction was completed, water (30 mL) was added for quenching, and a yellow solid was precipitated during the process. The obtained mixture was filtered, and the filter cake was washed with methanol. The filter cake was collected and dried to obtain 0.17 g of yellow solid, i.e. the title compound with a yield of 88%.

¹H NMR (400 MHz, DMSO) δ 9.64 (s, 1H), 8.52 (s, 1H), 7.78 (d, J=2.4 Hz, 1H), 7.74 (d, J=9.0 Hz, 1H), 6.98 (dd, J=9.0, 2.4 Hz, 1H), 3.77 (q, J=7.3 Hz, 2H), 3.33 (s, 1H), 1.12 (t, J=7.3 Hz, 3H).

### Step 4. Preparation of 4-(2-bromo-4-nitrophenyl) morpholine (8)

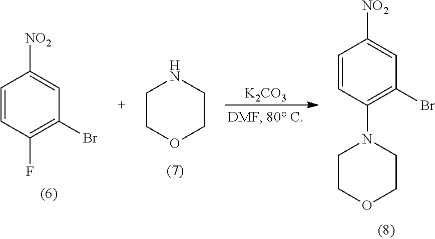

2-bromo-1-fluoro-4-nitrobenzene (5.0 g, 22.73 mmol) was dissolved in 50 mL DMF, then the solution was added with potassium carbonate (2.97 g, 45.46 mmol) and morpholine (2.97 g, 34.09 mmol). The reaction was heated in a preheated 80° C. oil bath for 4 hours, and the reaction process was monitored by TLC (developing agent: petroleum ether:ethyl acetate=5:1, v/v). After the reaction was complete, 80 mL of water was added, and the mixture was extracted with ethyl acetate. The organic phase was washed twice with saturated saline solution, then dried and concentrated. The residue was purified by rapid silica gel column chromatography (mobile phase: petroleum ether:ethyl acetate=5:1, v/v), and the effluent was collected one by one. The components containing the target substance were concentrated and dried to obtain 4.7 g of yellow solid with a yield of 72%.

¹H NMR (500 MHz, CDCl₃) δ 9.10 (d, J=2.8 Hz, 1H), 8.46 (dd, J=8.8, 2.8 Hz, 1H), 7.61 (d, J=8.8 Hz, 1H), 4.01-3.96 (m, 4H), 3.17-3.11 (m, 4H).

### Step 5. Preparation of 4-(4-nitro-2-(1H-pyrazol-4-yl)phenyl) morpholine (10)

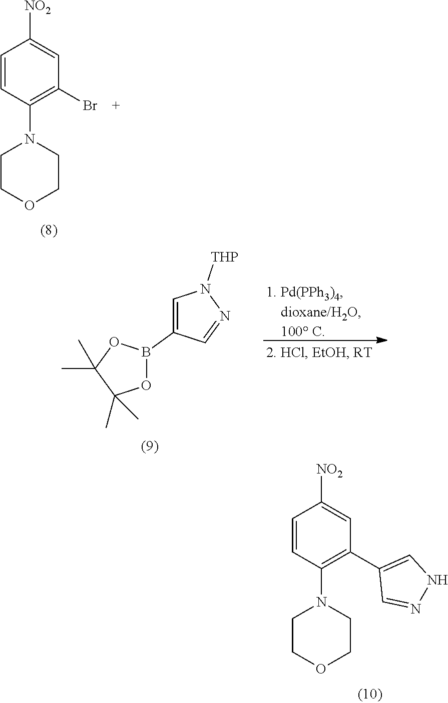

Intermediate 8 (3.0 g, 10.45 mmol), 1-(tetrahydro-2H-pyran-2-yl)-4-(4,4,5,5-tetramethyl-1,3,2-dioxaboran-2-yl)-1H pyrazole (3.78 g, 13.58 mmol), sodium carbonate (3.32 g, 31.35 mmol), and tetratriphenylphosphine palladium (1.21 g, 1.04 mmol) were added to a mixed solvent of 30 mL of water and 30 mL of dioxane, and the system was purged with nitrogen gas three times. The obtained mixture was reacted at 95° C. for 5 hours under a nitrogen atmosphere, and monitored by thin layer chromatography at 254 nm UV. The mobile phase was petroleum ether:ethyl acetate=5:1 (v/v). After the reaction was completed, the reaction solution was diluted with 80 mL of ethyl acetate and extracted with 100 ml of water. The organic phase was dried over anhydrous sodium sulfate and concentrated under reduced pressure. The residue was purified by rapid column chromatography using petroleum ether:ethyl acetate=2:1 (v/v) as the mobile phase. Collect the components containing the target substance, concentrate under reduced pressure, and obtain a yellow solid. The solid was dissolved in 15 mL of ethanol, add 3 mL of concentrated hydrochloric acid. The mixture was stirred at room temperature for 3 hours, and then quenched with 50 mL of saturated sodium bicarbonate aqueous solution, then extracted twice with 30 mL of ethyl acetate. The organic phases were combined, dried, and concentrated to obtain the target substance as a light yellow solid (2.1 g, 73%).

¹H NMR (500 MHz, CDCl₃) δ 8.18 (t, J=2.3 Hz, 1H), 8.15-8.09 (m, 3H), 7.10 (dd, J=8.9, 1.9 Hz, 1H), 3.81-3.76 (m, 4H), 3.03-2.96 (m, 4H).

### Step 6. Preparation of 3-(4-(2-morpholino-5-nitrophenyl)-1H-pyrazol-1-yl) propan-1-ol (12)

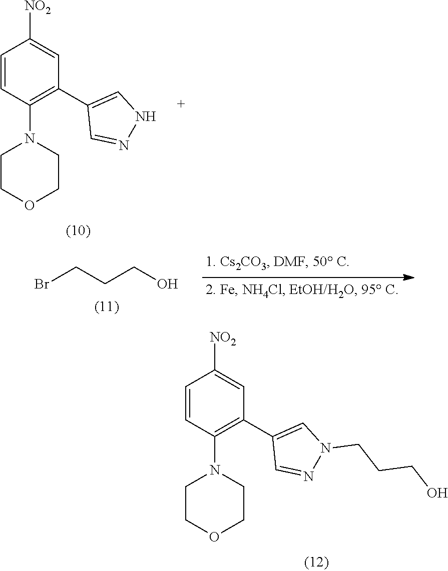 [SMILES: O=[N+]([O-])c1ccc(N2CCOCC2)c(-c2cn[nH]c2)c1 ; OCCCBr ; O=[N+]([O-])c1ccc(N2CCOCC2)c(-c2cnn(CCCO)c2)c1]

Intermediate 10 (0.50 g, 1.82 mmol), 3-bromopropanol (0.38 g, 2.73 mmol, 1.5 equivalents), and cesium carbonate (1.78 g, 5.47 mmol) were dissolved in 10 mL of DMF. The mixture was reacted at 50° C. for 3 hours and monitored by thin-layer chromatography at 254 nm UV. The mobile phase was dichloromethane:methanol=15:1 (v/v). After the reaction was complete, the reaction solution was diluted with 20 mL of ethyl acetate and extracted with 30 mL of water. The organic phase was washed with saturated saline solution (30 mL×2), dried with anhydrous sodium sulfate, and concentrated under reduced pressure. The residue was dissolved in a mixed solvent of 20 mL of ethanol and 10 mL of water, and reduced iron powder (0.41 g, 7.29 mmol) and ammonium chloride (0.39 g, 7.29 mmol) were added to it. The mixture was heated to 95° C. and reacted for 4 hours. During the process, thin-layer chromatography was used to monitor under 254 nm UV, and the mobile phase was dichloromethane:methanol=15:1 (v/v). After the reaction was completed, the reaction solution was filtered through diatomaceous earth and washed with ethyl acetate. The filtrate was extracted with ethyl acetate, and the organic phase was dried and concentrated. The residue was purified by rapid column chromatography using dichloromethane:methanol=15:1 (v/v) as the mobile phase. The components containing the target substance was collected, concentrated under reduced pressure, to obtain the target substance as a brown solid (0.42 g, 69%).

¹H NMR (500 MHz, CDCl₃) δ 8.00 (s, 1H), 7.92 (s, 1H), 6.95 (d, J=8.4 Hz, 1H), 6.76 (d, J=2.8 Hz, 1H), 6.58 (dd, J=8.4, 2.8 Hz, 1H), 4.32 (t, J=6.3 Hz, 2H), 3.78-3.72 (m, 4H), 3.66 (t, J=5.7 Hz, 2H), 2.94 (s, 2H), 2.84-2.79 (m, 4H), 2.12-2.02 (m, 2H), 1.31-1.23 (m, 1H).

### Step 7. Preparation of 3-(5-Chloro-2-((3-(1-(3-hydroxypropyl)-1H-pyrazol-4-yl)-4-morpholinylphenyl)amino) pyrimidin-4-yl)-1-(ethylsulfonyl)-1H-indole-6-ol (13)

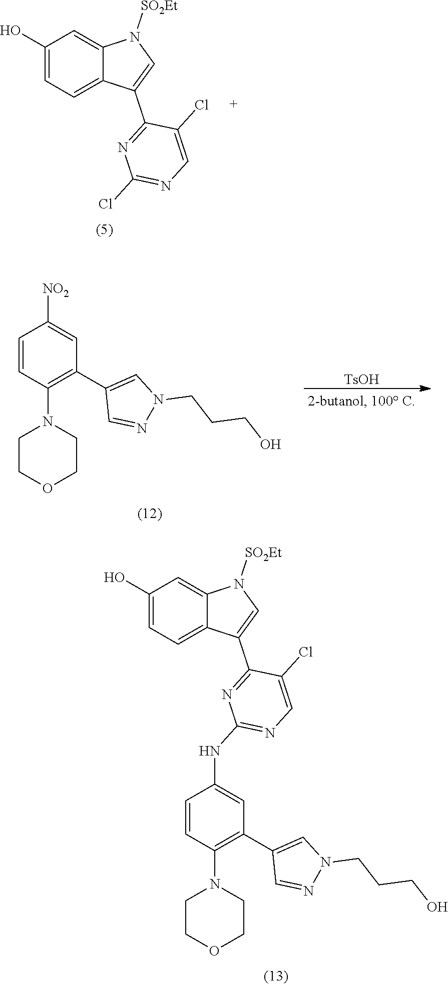

Intermediate 5 (0.30 g, 0.81 mmol), Intermediate 12 (0.24 g, 0.81 mmol), and p-toluenesulfonic acid (0.04 g, 0.24 mmol) were dissolved in 10 mL of sec-butanol, and the reaction was heated to 100° C. for 12 hours. During the process, thin-layer chromatography was used to monitor under 254 nm UV, and the mobile phase was dichloromethane:methanol=15:1 (v/v). After completed, the reaction was quenched with 20 mL of saturated sodium bicarbonate aqueous solution. The mixture was extracted twice with 15 mL of ethyl acetate, and the organic phases were combined, dried, and concentrated. The residue was purified by rapid column chromatography using dichloromethane:methanol=15:1 (v/v) as the mobile phase. The components containing the target substance was collected, concentrated under reduced pressure to obtain the target substance as a yellow solid (0.26 g, 51%).

¹H NMR (500 MHz, CDCl₃) δ 8.41 (s, 1H), 8.26 (d, J=5.6 Hz, 1H), 8.20-8.14 (m, 2H), 7.96 (s, 1H), 7.74 (s, 1H), 7.32 (d, J=2.1 Hz, 1H), 7.11 (d, J=8.5 Hz, 1H), 7.07 (d, J=5.6 Hz, 1H), 7.04 (dd, J=9.3, 2.7 Hz, 1H), 5.82 (d, J=8.3 Hz, 1H), 4.34-4.26 (m, 2H), 3.99 (s, 2H), 3.81 (t, J=4.6 Hz, 5H), 3.37 (q, J=7.4 Hz, 2H), 2.97-2.89 (m, 5H), 1.97-1.89 (m, 2H), 1.23 (t, J=7.4 Hz, 3H).

### Step 8. Preparation of (Z)-2⁵-Chloro-1¹-(ethylsulfonyl)-4⁴-morpholino-1¹H,5¹H-9-oxa-3-aza-1(3,6)-indola-2(4,2)-pyrimidina-5(4,1)-pyrazola-4(1,3)-benzenacyclononaphane (HC871)

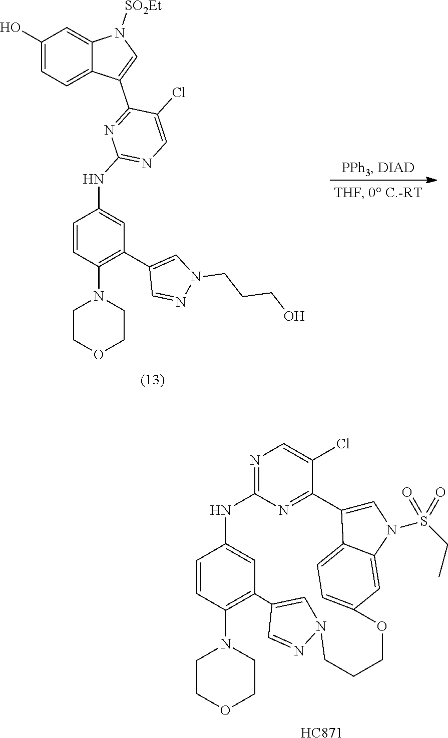

Intermediate 13 (0.10 g, 0.16 mmol) and triphenylphosphine (0.41 g, 1.57 mmol) were dissolved in 70 mL of THF. The mixture was cooled to 0° C., and slowly added DIAD (0.31 g, 1.57 mmol) at 0° C., followed with moving to room temperature for 5 hours. During the process, thin-layer chromatography was used to monitor under 254 nm UV, and the mobile phase was dichloromethane:methanol=15:1 (v/v). After the reaction was completed, the reaction solution was concentrated and the residue was purified by rapid column chromatography using a mobile phase of petroleum ether:ethyl acetate=1:1, v/v. The components containing the target substance was collected, concentrated under reduced pressure to obtain the target substance as a yellow solid (0.04 g, 41%).

¹H NMR (500 MHz, CDCl₃) δ 8.61 (d, J=2.7 Hz, 1H), 8.47 (s, 1H), 8.46 (s, 1H), 8.28 (s, 1H), 8.21 (d, J=8.9 Hz, 1H), 7.65 (d, J=2.2 Hz, 1H), 7.09 (d, J=8.5 Hz, 1H), 6.80 (dd, J=8.5, 2.7 Hz, 1H), 6.67 (s, 1H), 6.45 (dd, J=9.0, 2.2 Hz, 1H), 4.37 (t, J=5.2 Hz, 2H), 3.94 (t, J=5.8 Hz, 2H), 3.81 (t, J=4.5 Hz, 4H), 3.45 (q, J=7.3 Hz, 2H), 2.89 (t, J=4.5 Hz, 4H), 2.52 (p, J=5.4 Hz, 2H), 1.36 (t, J=7.4 Hz, 3H). ¹³C NMR (126 MHz, CDCl₃) δ 158.4, 158.0, 157.6, 156.3, 144.9, 139.7, 136.0, 135.7, 129.8, 129.5, 128.0, 123.9, 123.0, 120.1, 119.9, 117.9, 117.6, 117.2, 117.0, 112.2, 102.5, 67.3 (2C), 65.0, 52.1 (2C), 49.0, 47.4, 30.5, 8.1. HRMS (m/z): [M+H]⁺ calculated for C₃₀H₃₁ClN₇O₄S, 620.1841. found, 620.1846. HPLC purity: 97.17%.

### Embodiment 2

### Preparation of 2⁵-Chloro-4⁶-Methyl-4⁴-morpholino-1¹H,5¹H-9-oxa-3-aza-1(3,6)-Indole-2(4,2)-Pyrimidin-5(4,1)Pyrazol-4(1,3)-benzenacyclononaphane (HC8990)

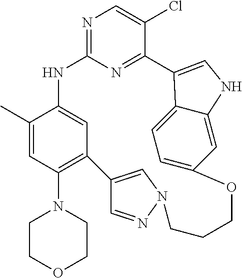 [SMILES: Cc1cc(N2CCOCC2)c2cc1Nc1ncc(Cl)c(n1)-c1c[nH]c3cc(ccc13)OCCCn1cc-2cn1]

The synthesis method followed Embodiment 1, where Intermediate 6 in step 4 was replaced with 1-bromo-2-fluoro-4-methyl-5-nitrobenzene, and Intermediate 4 in step 3 is replaced with Intermediate 3.

¹H NMR (400 MHz, CDCl₃) δ 8.77 (s, 1H), 8.43 (s, 1H), 8.35 (s, 1H), 8.29 (d, J=8.8 Hz, 1H), 8.06 (s, 1H), 7.04 (s, 1H), 7.00 (s, 1H), 6.97 (s, 1H), 6.82 (s, 1H), 6.29 (d, J=9.0 Hz, 1H), 4.36 (t, J=5.7 Hz, 2H), 3.93 (d, J=5.8 Hz, 2H), 3.86 (s, 3H), 3.82 (t, J=4.5 Hz, 4H), 2.91 (t, J=4.4 Hz, 5H), 2.51-2.43 (m, 2H), 2.42 (s, 4H).

### Embodiment 3

### Preparation of 2⁵-Chloro-1¹,4⁶-dimethyl-4⁴-morpholino-1¹H,5¹H-9-oxa-3-aza-1(3,6)-Indole-2(4,2)-Pyrimidin-5(4,1)-Pyrazolol-4(1,3)-benzenacyclononaphane (HC8991)

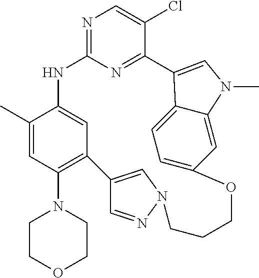

The synthesis method followed Embodiment 1, where Intermediate 6 in step 4 was replaced with 1-bromo-2-fluoro-4-methyl-5-nitrobenzene, and in Step 2, ethylsulfonyl chloride was replaced with iodomethane.

¹H NMR (400 MHz, CDCl₃) δ 8.77 (s, 1H), 8.51 (s, 1H), 8.42 (s, 1H), 8.38 (s, 1H), 8.28 (d, J=8.9 Hz, 1H), 8.18 (d, J=2.8 Hz, 1H), 7.10-7.05 (m, 2H), 6.97 (s, 1H), 6.81 (s, 1H), 6.31 (d, J=9.0 Hz, 1H), 4.34 (t, J=5.5 Hz, 2H), 3.92 (t, J=5.6 Hz, 2H), 3.85-3.73 (m, 4H), 2.98-2.83 (m, 4H), 2.50-2.44 (m, 2H), 2.42 (s, 3H), 1.28 (s, 3H).

### Embodiment 4

### Preparation of 2⁵-chloro-4⁵-cyclopentyl-1¹-ethylsulfonyl-4⁴-morpholino-1¹H,5¹H-9-oxa-3-aza-1(3,6)-indole-2(4,2)-pyrimidin-5(4,1)-pyrazol-4(1,3)-benzenacyclononaphane (HC9001)

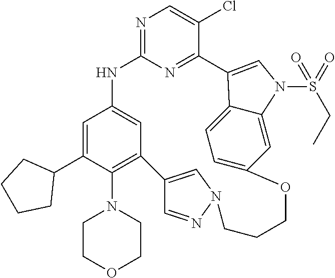 [SMILES: CCS(=O)(=O)n1cc2c3ccc(cc31)OCCCn1cc(cn1)-c1cc(cc(C3CCCC3)c1N1CCOCC1)Nc1ncc(Cl)c-2n1]

### Step 1. Preparation of 4-(2,6-dibromo-4-nitrophenyl) morpholine (15)

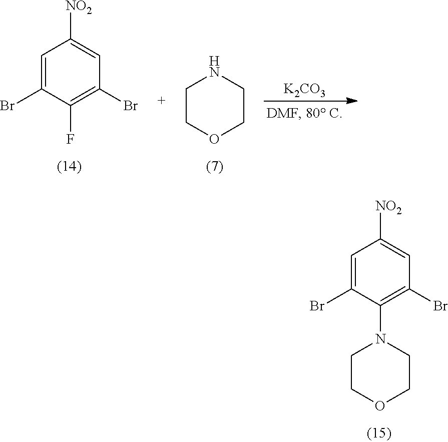

The synthesis method follows step 4 of Embodiment 1, and Intermediate 6 is replaced with 1,3-dibromo-2-fluoro-5-nitrobenzene. ¹H NMR (500 MHz, CDCl₃) δ 8.45-8.33 (m, 2H), 3.91-3.86 (m, 4H), 3.37-3.29 (m, 4H).

### Step 2. Preparation of 4-(2,6-dibromo-4-nitrophenyl) morpholine (16)

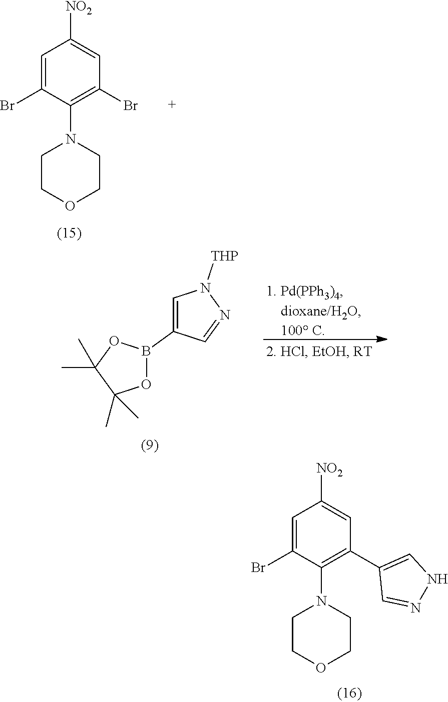

The synthesis method followed step 5 of Embodiment 1, and Intermediate 8 was replaced with Intermediate 15.

¹H NMR (500 MHz, cdcl₃) δ 8.41 (d, J=2.8 Hz, 1H), 8.09 (d, J=2.8 Hz, 1H), 7.73 (s, 2H), 7.72 (s, 1H), 3.77-3.74 (m, 4H), 3.04-2.94 (m, 4H).

### Step 3. Preparation of 3-(4-(3-(cyclopentan-1-en-1-yl)-2-morpholino-5-nitrophenyl)-1H-pyrazol-1-yl) propan-1-ol (18)

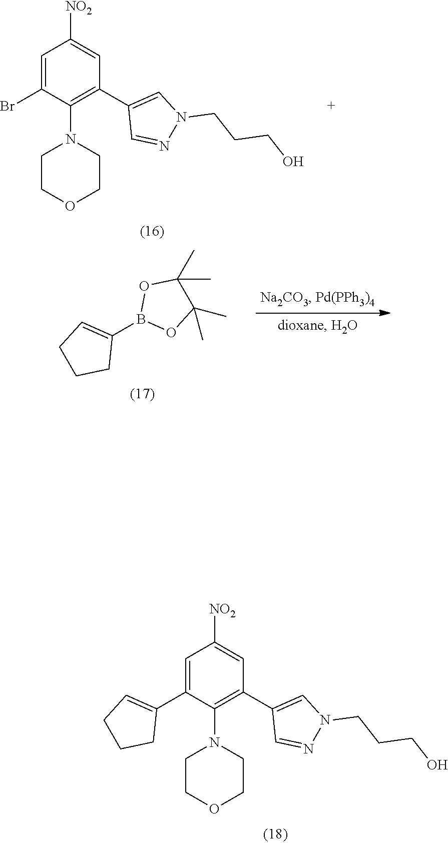 [SMILES: O=[N+]([O-])c1cc(Br)c(N2CCOCC2)c(-c2cnn(CCCO)c2)c1 ; CC1(C)OB(C2=CCCC2)OC1(C)C ; O=[N+]([O-])c1cc(C2=CCCC2)c(N2CCOCC2)c(-c2cnn(CCCO)c2)c1]

Intermediate 16 (0.3 g, 0.73 mmol), Intermediate 17 (0.21 g, 1.09 mmol), sodium carbonate (0.23 g, 2.19 mmol, 3 equivalents), and tetratriphenylphosphine palladium (0.1 g, 0.07 mmol, 0.1 equivalents) were added to a mixed solvent of 10 mL of water and 10 mL of dioxane. The system was purged with nitrogen gas three times. The obtained mixture was reacted at 95° C. for 5 hours under a nitrogen atmosphere, and monitored by thin-layer chromatography at 254 nm UV. The mobile phase was dichloromethane:methanol=30:1 (v/v). After the reaction was complete, the reaction solution was diluted with 20 mL of ethyl acetate and extracted with 50 mL of water. The organic phase was dried over anhydrous sodium sulfate and concentrated under reduced pressure. The residue was purified by rapid column chromatography using a mobile phase of dichloromethane:methanol=30:1, v/v. The components containing the target substance was collected, concentrated under reduced pressure to obtain a yellow solid (0.21 g, 72%).

¹H NMR (500 MHz, CDCl₃) δ 7.95 (dd, J=2.9, 1.4 Hz, 1H), 7.89 (dd, J=2.9, 1.4 Hz, 1H), 7.75 (s, 1H), 7.65 (s, 1H), 5.77-5.71 (m, 1H), 4.38 (t, J=6.4 Hz, 2H), 3.71-3.68 (m, 2H), 3.63-3.60 (m, 4H), 2.98-2.94 (m, 4H), 2.72-2.66 (m, 2H), 2.59-2.54 (m, 2H), 2.16-2.11 (m, 2H), 2.09-2.05 (m, 2H).

### Step 4. Preparation of 3-(4-(5-amino-3-cyclopentyl-2-morpholinophenyl)-1H-pyrazol-1-yl) propan-1-ol (19)

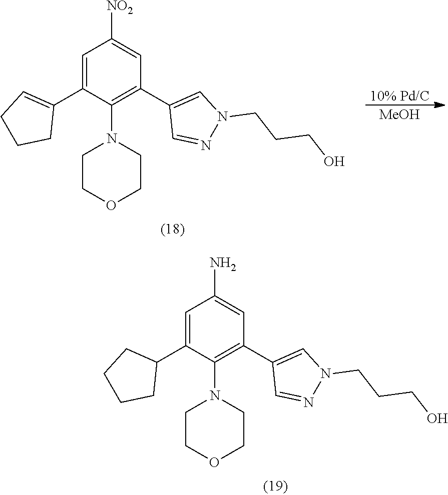 [SMILES: "MeOH" ; O=[N+]([O-])c1cc(C2=CCCC2)c(N2CCOCC2)c(-c2cnn(CCCO)c2)c1 ; "10% Pd/C" ; Nc1cc(-c2cnn(CCCO)c2)c(N2CCOCC2)c(C2CCCC2)c1]

Intermediate 18 (0.21 g, 0.53 mmol) was dissolved in 10 mL of methanol, 0.05 g of 10% palladium carbon was added, then the reaction system was purged with hydrogen gas and reacted at room temperature under a hydrogen atmosphere for 5 hours. After the reaction, the mixture was filtered through diatomite, washed with ethyl acetate, and the filtrate was concentrated to obtain a dark yellow foam like solid (0.16 g, 83%).

¹H NMR (500 MHz, CDCl₃) δ 7.60 (s, 1H), 7.55 (s, 1H), 6.59-6.53 (m, 1H), 6.48-6.44 (m, 1H), 4.36-4.27 (m, 2H), 3.76-3.70 (m, 2H), 3.67-3.58 (m, 4H), 3.44 (h, J=8.9, 8.4 Hz, 1H), 3.14-3.04 (m, 2H), 2.86-2.76 (m, 2H), 2.12-2.07 (m, 2H), 2.06-2.00 (m, 2H), 1.87-1.78 (m, 2H), 1.73-1.65 (m, 2H), 1.59-1.50 (m, 2H).

### Step 5. Preparation of 3-(5-Chloro-2-((3-(1-(3-hydroxypropyl)-1H-pyrazol-4-yl)-4-morpholinylphenyl)amino) pyrimidin-4-yl)-1-(ethylsulfonyl)-1H-indole-6-ol (20)

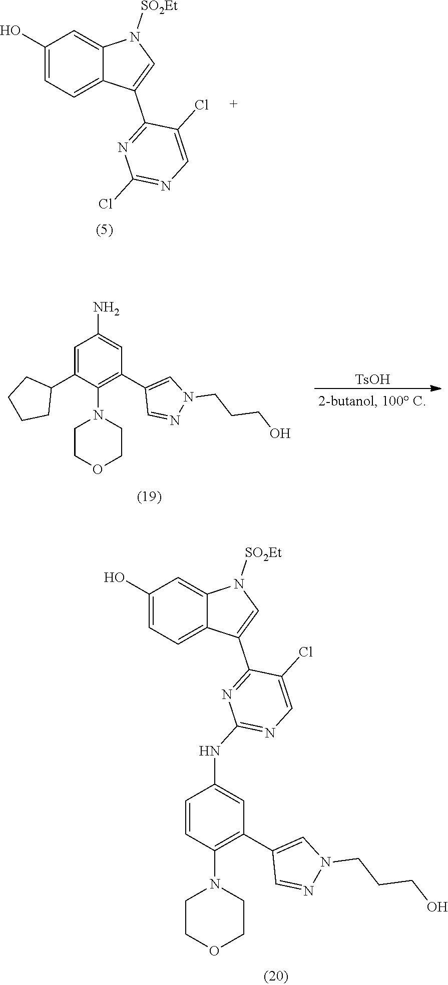

The synthesis method followed step 7 of Embodiment 1, and Intermediate 12 was replaced with Intermediate 19.

¹H NMR (500 MHz, CDCl₃) δ 8.39 (s, 1H), 8.36 (s, 1H), 8.04 (d, J=8.7 Hz, 1H), 7.99 (s, 1H), 7.80 (s, 1H), 7.47 (s, 1H), 7.38 (d, J=2.1 Hz, 1H), 6.92 (s, 1H), 6.18 (d, J=8.5 Hz, 1H), 4.31 (t, J=5.6 Hz, 2H), 3.94 (t, J=5.2 Hz, 2H), 3.80-3.70 (m, 4H), 3.67 (s, 4H), 3.50 (t, J=8.7 Hz, 1H), 3.38 (q, J=7.4 Hz, 2H), 3.16 (s, 2H), 2.82 (d, J=12.1 Hz, 2H), 1.88-1.85 (m, 2H), 1.74-1.71 (m, 2H), 1.59-1.55 (m, 2H), 1.29-1.27 (m, 3H).

### Step 6. Preparation of 2⁵-Chloro-4⁵-Cyclopentyl-1¹-Ethylsulfonyl-4⁴-morpholin-1¹H, 5¹H-9-oxa-3-aza-1(3,6)-Indole-2(4,2)-Pyrimidin-5(4,1)-Pyrazol-4(1,3)-benzenacyclononaphane (HC9001)

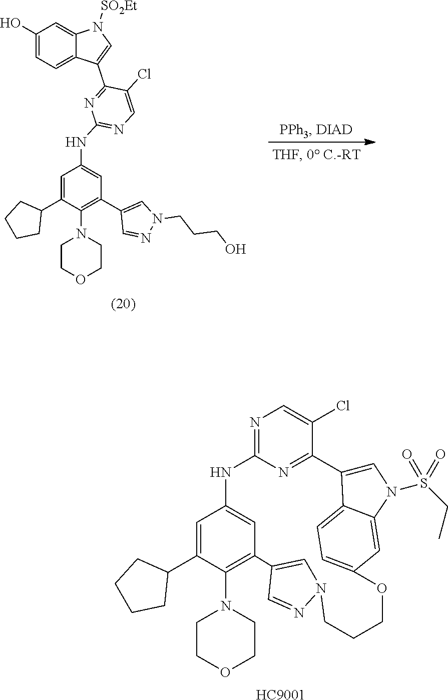

The synthesis method followed step 8 of Embodiment 1, and Intermediate 13 was replaced with Intermediate 20.

¹H NMR (500 MHz, CDCl₃) δ 8.36 (s, 1H), 8.25 (d, J=2.7 Hz, 1H), 8.14 (s, 1H), 8.04 (d, J=8.9 Hz, 1H), 7.80 (s, 1H), 7.47 (d, J=2.2 Hz, 1H), 7.29 (s, 1H), 6.70 (s, 1H), 6.68 (d, J=2.7 Hz, 1H), 6.21 (dd, J=9.0, 2.3 Hz, 1H), 4.39 (t, J=5.8 Hz, 2H), 3.97-3.90 (m, 2H), 3.68-3.58 (m, 4H), 3.48-3.39 (m, 1H), 3.34 (q, J=7.4 Hz, 2H), 3.12-3.04 (m, 2H), 2.81-2.73 (m, 2H), 2.34 (t, J=5.8 Hz, 2H), 2.07-1.98 (m, 2H), 1.85-1.75 (m, 2H), 1.71-1.64 (m, 2H), 1.53-1.45 (m, 2H), 1.25 (t, J=7.4 Hz, 3H). ¹³C NMR (126 MHz, CDCl₃) δ 157.4, 156.6, 156.2, 155.4, 147.1, 140.9, 139.1, 136.0, 134.9, 132.2, 128.8, 128.4, 122.6, 121.8, 120.4, 116.7, 116.2, 115.8, 115.5, 110.1, 101.1, 66.5 (2C), 63.6, 50.4 (2C), 47.8, 46.4, 39.2, 34.9, 28.9, 25.1, 7.1. HRMS (m/z): [M+H]⁺ calculated for C₃₅H₃₉ClN₇O₄S, 668.2467. found, 668.2471. HPLC purity: 98.42%.

### Embodiment 5

### Preparation of 2⁵-Chloro-1¹-ethylsulfonyl-4⁴-morpholino-4⁵-propenyl-1¹H,5¹H-9-oxa-3-aza-1(3,6)-indole-2(4,2)-pyrimidine-5(4,1)-pyrazole-4(1,3)-benzenacyclononaphane (HC9002)

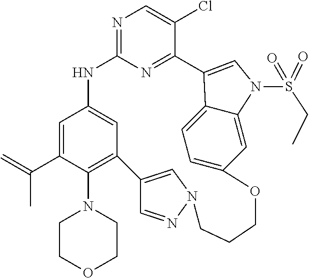 [SMILES: C=C(C)c1cc2cc(c1N1CCOCC1)-c1cnn(c1)CCCOc1ccc3c(cn(S(=O)(=O)CC)c3c1)-c1nc(ncc1Cl)N2]

The synthesis method followed Embodiment 4, where Intermediate 17 in step 3 was replaced with 4,4,5,5-tetramethyl-2-propene-2-propenyl-1,3,2-dioxaborane.

¹H NMR (500 MHz, CDCl₃) δ 8.45 (s, 1H), 8.39 (d, J=2.8 Hz, 1H), 8.24 (s, 1H), 8.12 (d, J=8.9 Hz, 1H), 8.01-7.96 (m, 1H), 7.56 (d, J=2.2 Hz, 1H), 7.27 (s, 1H), 6.87-6.82 (m, 1H), 6.59 (d, J=2.8 Hz, 1H), 6.27 (dd, J=9.0, 2.3 Hz, 1H), 5.20 (t, J=1.8 Hz, 1H), 4.93 (dd, J=2.1, 1.0 Hz, 1H), 4.50 (t, J=5.8 Hz, 2H), 4.10-3.99 (m, 2H), 3.65 (t, J=4.6 Hz, 4H), 3.43 (q, J=7.4 Hz, 2H), 2.98 (s, 4H), 2.49-2.37 (m, 2H), 2.17 (d, J=1.2 Hz, 3H), 1.34 (t, J=7.4 Hz, 3H). ¹³C NMR (126 MHz, CDCl₃) δ 158.4, 157.7, 157.3, 156.4, 145.9, 143.8, 141.3, 140.0, 136.0, 135.6, 132.0, 129.6, 129.4, 123.6, 122.8, 121.3, 119.0, 118.8, 117.8, 116.8, 115.6, 110.8, 102.0, 67.2 (2C), 64.5, 51.6 (2C), 48.9, 47.4, 29.8, 29.7, 25.5, 8.1. HRMS (m/z): [M+H]⁺ calculated for C₃₃H₃₅ClNO₄S, 660.2154. found, 660.2146. HPLC purity: 96.62%.

### Embodiment 6

### Preparation of 2⁵-Chloro-1¹-ethylsulfonyl-4⁴-morpholino-4⁵-2-methylpropan-1-en-1-yl-1¹H,5¹H-9-oxa-3-aza-1(3,6)-indole-2(4,2)-pyrimidine-5(4,1)-pyrazole-4(1,3)-benzenacyclononaphane (HC9003)

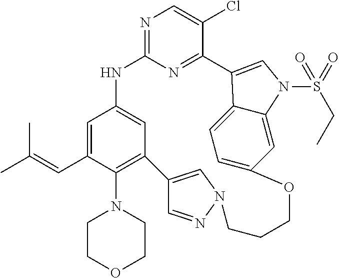 [SMILES: CCS(=O)(=O)n1cc2c3ccc(cc31)OCCCn1cc(cn1)-c1cc(cc(C=C(C)C)c1N1CCOCC1)Nc1ncc(Cl)c-2n1]

The synthesis method followed Embodiment 4, where Intermediate 17 in step 3 was replaced with 4,4,5,5-tetramethyl-2-(2-methylpropen-1-yl)-1,3,2-dioxaborane.

¹H NMR (400 MHz, DMSO-d₆) δ 9.77 (s, 1H), 8.63 (s, 1H), 8.19 (d, J=11.4 Hz, 2H), 8.05 (d, J=8.8 Hz, 1H), 7.69 (s, 1H), 7.36 (s, 2H), 6.96 (s, 1H), 6.38 (s, 1H), 6.12 (d, J=9.0 Hz, 1H), 4.39 (s, 2H), 4.06 (s, 2H), 3.74 (d, J=7.5 Hz, 2H), 3.53 (s, 4H), 2.76 (s, 4H), 2.17 (s, 2H), 1.91 (s, 3H), 1.73 (s, 3H), 1.14 (t, J=7.4 Hz, 3H).

### Embodiment 7

### Preparation of 2⁵-chloro-1¹-ethylsulfonyl-4⁴-morpholino-4⁵-2-methylpropyl-1¹H,5¹H-9-oxa-3-aza-1(3,6)-indole-2(4,2)-pyrimidin-5(4,1)-pyrazol-4(1,3)-benzenacyclononaphane (HC90032)

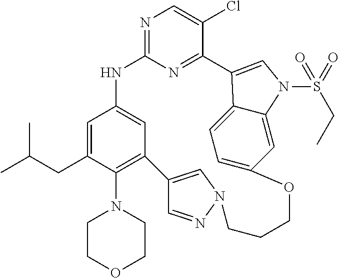 [SMILES: CCS(=O)(=O)n1cc2c3ccc(cc31)OCCCn1cc(cn1)-c1cc(cc(CC(C)C)c1N1CCOCC1)Nc1ncc(Cl)c-2n1]

The synthesis method followed Embodiment 4, wherein Intermediate 17 in step 3 was replaced with 4,4,5,5-tetramethyl-2-(2-methylpropen-1-yl)-1,3,2-dioxaborane.

¹H NMR (500 MHz, CDCl₃) δ 8.42 (s, 1H), 8.26 (d, J=2.8 Hz, 1H), 8.18 (s, 1H), 8.07 (d, J=8.9 Hz, 1H), 7.72 (s, 1H), 7.51 (d, J=2.2 Hz, 1H), 7.22 (s, 1H), 6.84 (s, 1H), 6.68 (d, J=2.8 Hz, 1H), 6.24 (dd, J=9.0, 2.3 Hz, 1H), 4.50 (t, J=6.0 Hz, 2H), 4.11-4.05 (m, 2H), 3.65 (s, 5H), 3.39 (q, J=7.4 Hz, 2H), 2.86 (d, J=4.7 Hz, 4H), 2.56 (d, J=7.2 Hz, 2H), 2.36 (q, J=5.9 Hz, 3H), 0.97 (s, 3H), 0.95 (s, 3H).

### Embodiment 8

### Preparation of 2⁵-chloro-4⁵-(E)-3,3-dimethylbut-1-en-1-yl)-1¹-ethylsulfonyl-4⁴-morpholin-1¹H,5¹H-9-oxa-3-aza-1(3,6)-indole-2(4,2)-pyrimidin-5(4,1)-pyrazolol-4 (1,3)-benzenacyclononaphane (HC9004)

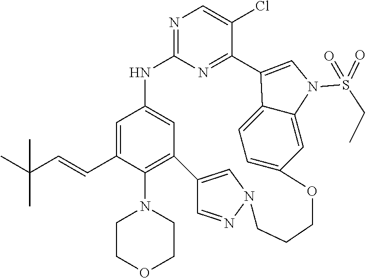 [SMILES: CCS(=O)(=O)n1cc2c3ccc(cc31)OCCCn1cc(cn1)-c1cc(cc(/C=C/C(C)(C)C)c1N1CCOCC1)Nc1ncc(Cl)c-2n1]

The synthesis method followed Embodiment 4, where Intermediate 17 in step 3 was replaced with 2-(3,3-dimethylbut-1-en-1-yl)-4,4,5,5-tetramethyl-1,3,2-dioxaborane.

¹H NMR (500 MHz, CDCl₃) δ 8.45 (s, 1H), 8.37 (d, J=2.7 Hz, 1H), 8.23 (s, 1H), 8.12 (d, J=8.9 Hz, 1H), 7.85 (s, 1H), 7.56 (d, J=2.2 Hz, 1H), 7.50-7.45 (m, 1H), 6.92 (d, J=2.7 Hz, 1H), 6.80 (s, 1H), 6.77 (d, J=16.1 Hz, 1H), 6.27 (dd, J=9.0, 2.3 Hz, 1H), 6.09 (d, J=16.1 Hz, 1H), 4.49 (t, J=5.8 Hz, 2H), 4.12-3.99 (m, 2H), 3.72 (t, J=4.6 Hz, 4H), 3.43 (q, J=7.4 Hz, 2H), 2.94 (s, 4H), 2.49-2.33 (m, 2H), 1.72 (s, 1H), 1.34 (t, J=7.4 Hz, 3H), 1.18 (s, 6H). ¹³C NMR (126 MHz, CDCl₃) δ 158.4, 157.8, 157.2, 156.4, 143.2, 141.4, 140.1, 138.8, 136.2, 136.0, 131.8, 129.7, 129.4, 123.7, 122.8, 122.8, 121.4, 118.6, 117.7, 116.8, 116.4, 110.9, 102.0, 67.6 (2C), 64.5, 51.4 (2C), 48.9, 47.4, 33.7, 29.9, 29.7 (3C), 8.1. HRMS (m/z): [M+H]⁺ calculated for C₃₆H₄₁ClN₇O₄S, 702.2624. found, 702.2624. HPLC purity: 98.11%.

### Embodiment 9

### Preparation of 2⁵-chloro-4⁵-(E)-3,3-dimethylbutyl)-1¹-ethylsulfonyl-4⁴-morpholin-1¹H,5¹H-9-oxa-3-aza-1(3,6)-indole-2(4,2)-pyrimidin-5(4,1)-pyrazolol-4 (1,3)-benzenacyclononaphane (HC90042)

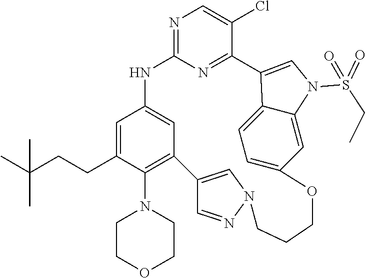 [SMILES: CCS(=O)(=O)n1cc2c3ccc(cc31)OCCCn1cc(cn1)-c1cc(cc(CCC(C)(C)C)c1N1CCOCC1)Nc1ncc(Cl)c-2n1]

The synthesis method followed Embodiment 4, where Intermediate 17 in step 3 was replaced with 2-(3,3-dimethylbut-1-en-1-yl)-4,4,5,5-tetramethyl-1,3,2-dioxaborane.

¹H NMR (500 MHz, CDCl₃) δ 8.45 (s, 1H), 8.29 (d, J=2.7 Hz, 1H), 8.22 (s, 1H), 8.11 (d, J=9.0 Hz, 1H), 7.80 (s, 1H), 7.55 (d, J=2.2 Hz, 1H), 6.85 (s, 1H), 6.72 (d, J=2.8 Hz, 1H), 6.33-6.23 (m, 1H), 4.52 (t, J=6.0 Hz, 2H), 4.07 (t, J=5.7 Hz, 2H), 3.71 (q, J=6.2, 4.5 Hz, 4H), 3.42 (q, J=7.3 Hz, 2H), 3.04-2.93 (m, 2H), 2.92-2.81 (m, 2H), 2.79-2.61 (m, 2H), 2.41 (p, J=5.8 Hz, 2H), 1.63 (s, 2H), 1.58-1.47 (m, 2H), 1.34 (t, J=7.4 Hz, 3H), 1.18 (s, 1H), 1.04 (s, 6H). ¹³C NMR (126 MHz, CDCl₃) δ 158.4, 157.7, 157.1, 156.4, 144.0, 142.4, 140.1, 136.4, 135.9, 132.9, 129.6, 129.4, 123.6, 122.8, 121.8, 119.3, 118.0, 117.8, 116.8, 110.8, 102.0, 67.5 (2C), 64.5, 51.7 (2C), 48.8, 47.5, 46.4, 30.8, 29.8, 29.7, 29.6 (2C), 27.3, 8.1. HRMS (m/z): [M+H]⁺ calculated for C₃₆H₄₃ClN₇O₄S, 704.2780. found, 704.2781. HPLC purity: 95.19%.

### Embodiment 10

### Preparation of 2⁵-chloro-1¹-ethylsulfonyl-4⁶-methyl-4⁴-morpholino-1¹H,5¹H-9-oxa-3-aza-1(3,6)-indole-2(4,2)-pyrimidin-5(4,1)-pyrazol-4(1,3)-benzenacyclononaphane (HC916)

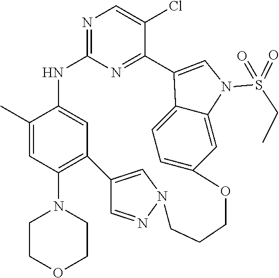 [SMILES: CCS(=O)(=O)n1cc2c3ccc(cc31)OCCCn1cc(cn1)-c1cc(c(C)cc1N1CCOCC1)Nc1ncc(Cl)c-2n1]

The synthesis method followed Embodiment 1, where Intermediate 6 in step 4 was replaced with 1-bromo-2-fluoro-4-methyl-5-nitrobenzene.

¹H NMR (500 MHz, CDCl₃) δ 8.56 (s, 1H), 8.44 (s, 1H), 8.33 (s, 1H), 8.27 (s, 1H), 8.17 (d, J=8.9 Hz, 1H), 7.58 (d, J=2.2 Hz, 1H), 7.10 (s, 1H), 6.94 (s, 1H), 6.80 (s, 1H), 6.32 (dd, J=9.0, 2.3 Hz, 1H), 4.36 (t, J=5.5 Hz, 2H), 3.98-3.89 (m, 2H), 3.78 (t, J=4.5 Hz, 4H), 3.42 (q, J=7.3 Hz, 2H), 2.87 (t, J=4.6 Hz, 4H), 2.47 (q, J=5.6 Hz, 2H), 2.40 (s, 3H), 1.32 (t, J=7.3 Hz, 3H). ¹³C NMR (126 MHz, CDCl₃) δ 158.4, 157.8, 157.7, 156.3, 144.6, 139.5, 135.9, 133.7, 129.5, 129.5, 125.6, 124.3, 123.9, 123.0, 121.6, 120.0, 118.9, 117.6, 116.9, 112.0, 102.3, 67.3 (2C), 65.1, 52.1 (2C), 48.9, 47.6, 30.5, 18.0, 8.1. HRMS (m/z): [M+H]⁺ calculated for C₃₁H₃₃ClN₇O₄S, 634.1998. found, 634.1999. HPLC purity: 99.36%.

### Embodiment 11

### Preparation of 2⁵-chloro-1¹-ethylsulfonyl-7-methyl-4⁴-morpholino-1¹H, 5¹H-9-oxa-3-aza-1(3,6)-indole-2(4,2)-pyrimidine-5(4,1)-pyrazole-4(1,3)-benzenacyclononaphane (HC9281)

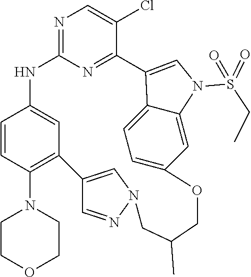 [SMILES: CCS(=O)(=O)n1cc2c3ccc(cc31)OCC(C)Cn1cc(cn1)-c1cc(ccc1N1CCOCC1)Nc1ncc(Cl)c-2n1]

The synthesis method followed Embodiment 1, where Intermediate 11 in step 6 was replaced with 3-bromo-2-methyl-1-propanol.

¹H NMR (500 MHz, CDCl₃) δ 8.53 (d, J=2.7 Hz, 1H), 8.43 (s, 1H), 8.39 (s, 1H), 8.26 (s, 1H), 8.20 (d, J=8.9 Hz, 1H), 7.60 (d, J=2.2 Hz, 1H), 7.29 (s, 1H), 7.06 (d, J=8.6 Hz, 1H), 6.79 (dd, J=8.5, 2.7 Hz, 1H), 6.71 (s, 1H), 6.40 (dd, J=9.0, 2.3 Hz, 1H), 4.37 (dd, J=12.8, 3.1 Hz, 1H), 4.17 (dd, J=12.8, 2.7 Hz, 1H), 3.92-3.85 (m, 1H), 3.83-3.68 (m, 4H), 3.65-3.57 (m, 1H), 3.42 (qd, J=7.3, 2.2 Hz, 2H), 2.99-2.90 (m, 2H), 2.83-2.75 (m, 2H), 2.75-2.68 (m, 1H), 1.37-1.25 (m, 6H). ¹³C NMR (126 MHz, CDCl₃) δ 158.4, 157.9, 157.7, 156.3, 145.1, 139.7, 135.9, 135.6, 129.5, 129.5, 128.0, 123.9, 123.1, 120.0, 119.9, 118.3, 117.6, 117.5, 117.0, 112.7, 102.7, 70.2, 67.3 (2C), 53.6 (2C), 52.1, 48.9, 35.5, 15.7, 8.1. HRMS (m/z): [M+H]⁺ calculated for C₃₁H₃₃ClN₇O₄S, 634.1998. found, 634.1994. HPLC purity: 99.02%.

### Embodiment 12

### Preparation of (Z)-5′-Chloro-1′(Ethylsulfonyl)-4′-morpholino spiro [Cyclopropane-1,7′-9-oxa-3-aza-1(3,6)-indole-2(4,2)-pyrimidine-5(4,1)-pyrazolol-4(1,3)-benzenacyclononaphane] (HC9282)

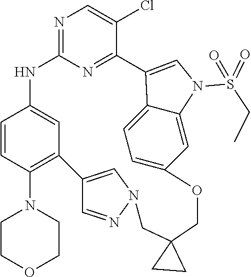 [SMILES: CCS(=O)(=O)n1cc2c3ccc(cc31)OCC1(CC1)Cn1cc(cn1)-c1cc(ccc1N1CCOCC1)Nc1ncc(Cl)c-2n1]

The synthesis method followed Embodiment 1, where Intermediate 11 in step 6 was replaced with 1-(bromomethyl)cyclopropylmethanol.

¹H NMR (500 MHz, CDCl₃) δ 8.57 (d, J=2.7 Hz, 1H), 8.50 (s, 1H), 8.37 (s, 1H), 8.20 (s, 1H), 8.15 (d, J=8.9 Hz, 1H), 7.63 (d, J=2.2 Hz, 1H), 7.21 (s, 1H), 7.01 (d, J=8.6 Hz, 1H), 6.70 (dd, J=8.6, 2.7 Hz, 1H), 6.51 (s, 1H), 6.47 (dd, J=8.9, 2.3 Hz, 1H), 4.03 (s, 2H), 3.79-3.69 (m, 4H), 3.59 (s, 2H), 3.37 (q, J=7.4 Hz, 2H), 2.84-2.71 (m, 4H), 1.27 (t, J=7.4 Hz, 3H), 0.86 (d, J=4.5 Hz, 2H), 0.81 (d, J=4.5 Hz, 2H). ¹³C NMR (126 MHz, CDCl₃) δ 157.4, 156.9, 156.6, 155.2, 143.8, 139.2, 134.9, 134.8, 128.9, 128.5, 127.1, 122.8, 122.2, 119.3, 118.5, 116.5, 116.5, 116.0, 116.0, 111.9, 102.1, 72.2, 66.2 (2C), 55.1, 51.2 (2C), 47.9, 21.1, 9.6, 7.1 (2C). HRMS (m/z): [M+H]⁺ calculated for C₃₂H₃₃ClN₇O₄S, 646.1998. found, 646.1989. HPLC purity: 95.96%.

### Embodiment 13

### Preparation of 2⁵-Chloro-1¹-ethylsulfonyl-4⁶-methoxy-4⁴-morpholin-1¹H,5¹H-9-oxa-3-aza-1(3,6)-indole-2(4,2)-pyrimidine-5(4,1)-pyrazole-4(1,3)-benzenacyclononaphane (HC9284)

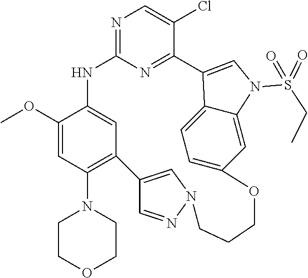 [SMILES: CCS(=O)(=O)n1cc2c3ccc(cc31)OCCCn1cc(cn1)-c1cc(c(OC)cc1N1CCOCC1)Nc1ncc(Cl)c-2n1]

The synthesis method followed Embodiment 1, where Intermediate 6 in step 4 was replaced with 1-bromo-2-fluoro-4-methoxy-5-nitrobenzene.

¹H NMR (500 MHz, CDCl₃) δ 8.56 (s, 1H), 8.38 (s, 1H), 8.35 (s, 1H), 8.17 (s, 1H), 8.12 (d, J=8.9 Hz, 1H), 7.89 (s, 1H), 7.56 (d, J=2.2 Hz, 1H), 6.66 (s, 1H), 6.45 (s, 1H), 6.38 (dd, J=9.0, 2.2 Hz, 1H), 4.27 (t, J=5.2 Hz, 2H), 3.90 (s, 3H), 3.83 (t, J=5.8 Hz, 2H), 3.73 (dd, J=6.0, 3.2 Hz, 4H), 3.36 (q, J=7.4 Hz, 2H), 2.86-2.74 (m, 4H), 2.48-2.38 (m, 2H), 1.27 (t, J=7.4 Hz, 3H). ¹³C NMR (126 MHz, CDCl₃) δ 158.4, 158.1, 157.6, 156.2, 146.6, 144.2, 139.6, 136.0, 129.4, 129.4, 125.4, 123.9, 123.1, 119.7, 119.7, 117.6, 117.4, 117.1, 112.2, 102.5, 102.2, 67.3 (2C), 65.0, 56.0, 52.2 (2C), 48.9, 47.2, 30.5, 8.1. HRMS (m/z): [M+H]⁺ calculated for C₃₁H₃₃ClN₇O₅S, 650.1947. found, 650.1939. HPLC purity: 99.32%.

### Embodiment 14

### Preparation of 2⁵-chloro-1¹-ethylsulfonyl-4⁶-methyl-4⁴-(4-methylpiperazin-1-yl)-1¹H,5¹H-9-oxa-3-aza-1(3,6)-indole-2(4,2)-pyrimidine-5(4,1)-pyrazole-4(1,3)-benzenacyclononaphane (HC9452)

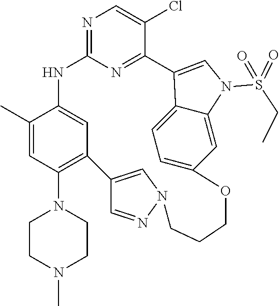 [SMILES: CCS(=O)(=O)n1cc2c3ccc(cc31)OCCCn1cc(cn1)-c1cc(c(C)cc1N1CCN(C)CC1)Nc1ncc(Cl)c-2n1]

The synthesis method followed Embodiment 1. In Step 4, Intermediate 6 was replaced with 1-bromo-2-fluoro-4-methyl-5-nitrobenzene, and Intermediate 7 was replaced with N-methylpiperazine.

¹H NMR (500 MHz, CDCl₃) δ 8.53 (s, 1H), 8.37 (s, 1H), 8.18 (s, 1H), 8.07 (d, J=8.8 Hz, 1H), 7.54 (d, J=2.2 Hz, 2H), 7.15 (s, 1H), 6.91 (s, 1H), 6.59 (s, 1H), 6.30 (dd, J=9.0, 2.2 Hz, 1H), 4.32-4.23 (m, 2H), 4.11-3.99 (m, 2H), 3.90-3.81 (m, 2H), 3.39-3.28 (m, 4H), 3.00 (t, J=4.6 Hz, 4H), 2.54 (s, 3H), 2.45-2.37 (m, 2H), 2.32 (s, 3H), 1.97-1.91 (m, 3H).

### Embodiment 15

### Preparation of 1¹-ethylsulfonyl-4⁶-methyl-4⁴-(4-methylpiperazin-1-yl)-1¹H,5¹H-9-oxa-3-aza-1(3,6)-indole-2(4,2)-pyrimidine-5(4,1)-pyrazole-4(1,3)-benzenacyclononaphane (HC950)

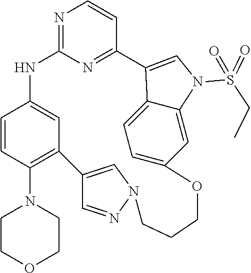 [SMILES: CCS(=O)(=O)n1cc2c3ccc(cc31)OCCCn1cc(cn1)-c1cc(ccc1N1CCOCC1)Nc1nccc-2n1]

The synthesis method followed Embodiment 1, wherein Intermediate 1 in Step 1 was replaced with 2,4-dichloropyrimidine.

¹H NMR (500 MHz, DMSO-d₆) δ 9.51 (s, 1H), 8.45 (d, J=5.2 Hz, 1H), 8.35 (d, J=2.4 Hz, 1H), 8.32 (t, J=4.4 Hz, 2H), 8.03 (s, 1H), 7.84 (s, 1H), 7.40 (d, J=5.2 Hz, 1H), 7.28 (d, J=2.2 Hz, 1H), 7.13-7.03 (m, 2H), 5.78 (dd, J=9.1, 2.3 Hz, 1H), 4.44-4.30 (m, 2H), 4.10-4.00 (m, 2H), 3.78-3.62 (m, 6H), 2.86-2.73 (m, 4H), 2.21-2.11 (m, 2H), 1.13 (t, J=7.3 Hz, 3H). ¹³C NMR (126 MHz, DMSO-d₆) δ 160.8, 160.5, 158.9, 156.4, 145.0, 138.9, 136.8, 136.5, 130.1, 128.2, 127.5, 124.4, 121.9, 121.8, 120.7, 119.7, 119.7, 119.3, 110.2, 108.5, 101.8, 66.8 (2C), 64.8, 52.2 (2C), 48.8, 48.1, 30.3, 8.3. HRMS (m/z): [M+H]⁺ calculated for C₃₀H₃₂N₇O₄S, 586.2231. found, 586.2221. HPLC purity: 97.67%.

### Embodiment 16

### Preparation of (Z)-1¹-Ethylsulfonyl-4⁶-methyl-4⁴-morpholino-1¹H,5¹H-9-oxa-3-aza-1 (3,6)-indole-2(4,2)-pyrimidine-5(4,1) pyrazol-4(1,3)-benzenacyclononaphane (HC950M)

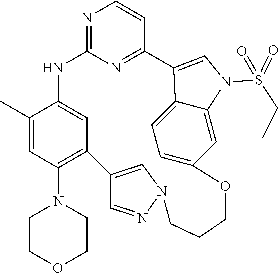 [SMILES: CCS(=O)(=O)n1cc2c3ccc(cc31)OCCCn1cc(cn1)-c1cc(c(C)cc1N1CCOCC1)Nc1nccc-2n1]

The synthesis method followed Embodiment 1, wherein Intermediate 1 in Step 1 was replaced with 2,4-dichloropyrimidine, and Intermediate 6 in Step 4 was replaced with 1-bromo-2-fluoro-4-methyl-5-nitrobenzene.

¹H NMR (500 MHz, DMSO-d₆) δ 8.66 (s, 1H), 8.49 (s, 1H), 8.40 (d, J=5.2 Hz, 1H), 8.30-8.22 (m, 2H), 7.91 (s, 1H), 7.89 (s, 1H), 7.33 (d, J=5.2 Hz, 1H), 7.15 (d, J=2.3 Hz, 1H), 6.92 (s, 1H), 5.39 (dd, J=9.2, 2.4 Hz, 1H), 4.34 (t, J=7.1 Hz, 2H), 4.29-4.21 (m, 2H), 3.90-3.70 (m, 4H), 3.63 (q, J=7.3 Hz, 2H), 2.90-2.72 (m, 4H), 2.30 (s, 3H), 2.08 (q, J=7.8 Hz, 2H), 1.07 (t, J=7.3 Hz, 3H). ¹³C NMR (126 MHz, DMSO-d₆) δ 161.7, 160.6, 158.8, 156.1, 146.3, 138.4, 136.6, 133.4, 130.4, 130.1, 127.8, 127.5, 124.7, 124.6, 121.8, 121.2, 120.6, 119.0, 109.0, 108.4, 101.0, 66.6, 65.4, 52.1, 48.7, 48.6, 31.0, 18.4, 8.2. [M+H]⁺ calculated for C₃₁H₃₄N₇O₄S, 600.2387; found, 600.2378. HPLC purity: 99.40%.

### Embodiment 17

### Preparation of (Z)-1¹-Ethylsulfonyl-4⁶-methoxy-4⁴-morpholin-1¹H,5¹H-9-oxa-3-aza-1 (3,6)-indole-2(4,2)-pyrimidine-5(4,1) pyrazole-4(1,3)-benzenacyclononaphane (HC9591)

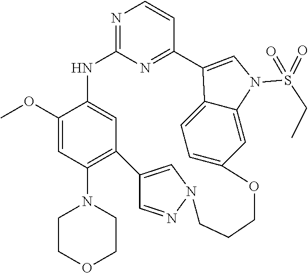 [SMILES: CCS(=O)(=O)n1cc2c3ccc(cc31)OCCCn1cc(cn1)-c1cc(c(OC)cc1N1CCOCC1)Nc1nccc-2n1]

The synthesis method followed Embodiment 1, wherein Intermediate 1 in Step 1 was replaced with 2,4-dichloropyrimidine, and Intermediate 6 in Step 4 was replaced with 1-bromo-2-fluoro-4-methoxy-5-nitrobenzene.

¹H NMR (500 MHz, CDCl₃) δ 8.79 (s, 1H), 8.44 (d, J=5.1 Hz, 1H), 8.42 (s, 1H), 8.35 (d, J=8.9 Hz, 1H), 7.90 (s, 1H), 7.83 (s, 1H), 7.53 (d, J=2.2 Hz, 1H), 7.01 (d, J=5.1 Hz, 1H), 6.83 (s, 1H), 6.75 (s, 1H), 6.21 (dd, J=9.0, 2.3 Hz, 1H), 4.44 (t, J=5.5 Hz, 2H), 3.98 (s, 3H), 3.95-3.89 (m, 2H), 3.87-3.76 (m, 4H), 3.42 (q, J=7.4 Hz, 2H), 2.98-2.84 (m, 4H), 2.51-2.43 (m, 2H), 1.34 (t, J=7.4 Hz, 3H). ¹³C NMR (126 MHz, CDCl₃) δ 160.5, 159.9, 158.5, 157.5, 146.9, 144.2, 139.4, 136.9, 129.4, 126.1, 125.7, 124.3, 122.2, 120.7, 120.3, 119.6, 118.6, 110.9, 108.2, 102.2, 102.0, 67.3 (2C), 64.1, 56.0, 52.2 (2C), 48.8, 47.2, 30.0, 8.1. [M+H]⁺ calculated for C₃₁H₃₄N₇O₄S, 616.2337. found, 616.2340. HPLC purity: 98.31%.

### Embodiment 18

### Preparation of (Z)-1¹-ethylsulfonyl-4⁶-methoxy-4⁴-(4-methylpiperazin-1-yl)-1¹H,5¹H-9-oxa-3-aza-1(3,6)-indole-2(4,2)-pyrimidine-5(4,1) pyrazole-4(1,3)-benzenacyclononaphane (HC9592)

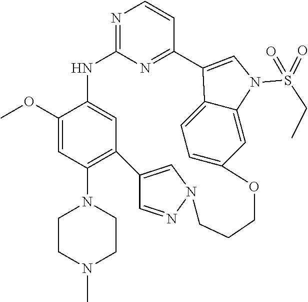 [SMILES: CCS(=O)(=O)n1cc2c3ccc(cc31)OCCCn1cc(cn1)-c1cc(c(OC)cc1N1CCN(C)CC1)Nc1nccc-2n1]

The synthesis method followed Embodiment 1, wherein Intermediate 1 in Step 1 was replaced with 2,4-dichloropyrimidine, Intermediate 6 in Step 4 was replaced with 1-bromo-2-fluoro-4-methoxy-5-nitrobenzene, and Intermediate 7 was replaced with N-methylpiperazine.

¹H NMR (500 MHz, DMSO) δ 10.40 (s, 1H), 9.69 (s, 1H), 8.35 (d, J=4.5 Hz, 2H), 8.26 (s, 1H), 8.21-8.10 (m, 2H), 7.94 (s, 1H), 7.84 (s, 1H), 7.36 (d, J=5.3 Hz, 1H), 7.27 (d, J=2.2 Hz, 1H), 6.81 (s, 1H), 6.53 (s, 1H), 4.26 (t, J=6.6 Hz, 2H), 3.85 (s, 3H), 3.69-3.62 (m, 2H), 3.59 (t, J=6.3 Hz, 2H), 3.34 (s, 3H), 3.21-3.03 (m, 4H), 2.93-2.61 (m, 4H), 2.23 (p, J=6.6 Hz, 2H), 1.09 (t, J=7.2 Hz, 3H).

### Embodiment 19

### Preparation of (Z)-1¹-ethylsulfonyl-4⁶-methoxy-4⁴-(4-methylpiperazin-1-yl) piperidin-1-yl)-1¹H,5¹H-9-oxa-3-aza-1(3,6)-indole-2(4,2)-pyrimidine-5(4,1)-pyrazolol-4(1,3)-benzenacyclononaphane (HC9594)

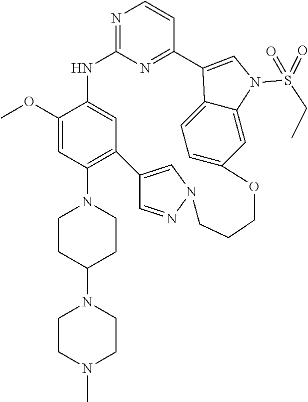 [SMILES: CCS(=O)(=O)n1cc2c3ccc(cc31)OCCCn1cc(cn1)-c1cc(c(OC)cc1N1CCC(N3CCN(C)CC3)CC1)Nc1nccc-2n1]

The synthesis method followed Embodiment 1, where Intermediate 1 in Step 1 was replaced with 2,4-dichloropyrimidine, Intermediate 6 in Step 4 was replaced with 1-bromo-2-fluoro-4-methoxy-5-nitrobenzene, and Intermediate 7 was replaced with 1-methyl-4-piperidin-4-ylpiperazine.

¹H NMR (500 MHz, CDCl₃) δ 8.75 (s, 1H), 8.44 (d, J=5.1 Hz, 1H), 8.35 (d, J=8.9 Hz, 1H), 7.89 (s, 1H), 7.53 (d, J=2.2 Hz, 1H), 7.01 (d, J=5.1 Hz, 1H), 6.89 (s, 1H), 6.72 (s, 1H), 6.22 (s, 1H), 4.43 (t, J=5.6 Hz, 2H), 3.96 (s, 3H), 3.42 (q, J=7.3 Hz, 2H), 3.29-3.18 (m, 2H), 3.16-2.74 (m, 6H), 2.70-2.55 (m, 3H), 2.50-2.38 (m, 2H), 2.12-1.67 (m, 7H), 1.34 (t, J=7.4 Hz, 3H).

### Embodiment 20

### Preparation of (Z)-4⁶-ethoxy-1¹-ethylsulfonyl-4⁴-morpholino-1¹H,5¹H-9-oxa-3-aza-1(3,6)-indole-2(4,2)-pyrimidine-5(4,1)-pyrazole-4(1,3)-benzenacyclononaphane (HC9596)

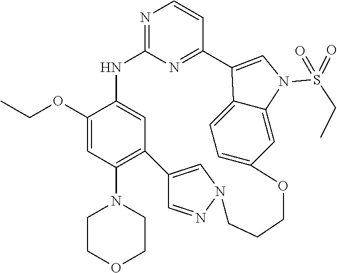 [SMILES: CCOc1cc(N2CCOCC2)c2cc1Nc1nccc(n1)-c1cn(S(=O)(=O)CC)c3cc(ccc13)OCCCn1cc-2cn1]

The synthesis method followed Embodiment 1, wherein Intermediate 1 in Step 1 was replaced with 2,4-dichloropyrimidine, and Intermediate 6 in Step 4 is replaced with 1-bromo-2-fluoro-4-ethoxy-5-nitrobenzene.

¹H NMR (500 MHz, CDCl₃) δ 8.81 (s, 1H), 8.44 (d, J=5.1 Hz, 1H), 8.42 (s, 1H), 8.35 (d, J=8.9 Hz, 1H), 7.92 (s, 1H), 7.84 (s, 1H), 7.54 (d, J=2.2 Hz, 1H), 7.01 (d, J=5.1 Hz, 1H), 6.82 (s, 1H), 6.74 (s, 1H), 6.23 (dd, J=8.9, 2.3 Hz, 1H), 4.43 (t, J=5.5 Hz, 2H), 4.21 (q, J=7.0 Hz, 2H), 3.94-3.87 (m, 2H), 3.86-3.72 (m, 4H), 3.42 (q, J=7.4 Hz, 2H), 2.99-2.81 (m, 4H), 2.47 (p, J=5.7 Hz, 2H), 1.55 (t, J=6.9 Hz, 3H), 1.34 (t, J=7.4 Hz, 3H). ¹³C NMR (126 MHz, CDCl₃) δ 160.5, 159.8, 158.5, 157.5, 146.2, 144.1, 139.4, 136.9, 129.4, 126.1, 125.8, 124.3, 122.2, 120.7, 120.3, 119.5, 118.6, 110.9, 108.2, 103.1, 102.0, 67.3 (2C), 64.6, 64.1, 52.2 (2C), 48.8, 47.2, 30.0, 15.0, 8.1. [M+H]⁺ calculated for C₃₁H₃₄N₇O₄S, 630.2493. found, 630.2492. HPLC purity: 98.87%.

### Embodiment 21

### Preparation of 2⁵-Chloro-1¹-Ethylsulfonyl-4⁴-morpholino-1¹H,5¹H-10-oxa-3-aza-1(3,6)-Indole-2(4,2)-Pyrimidin-5(4,1)-Pyrazol-4(1,3)-Phenyldecane (HC8714)

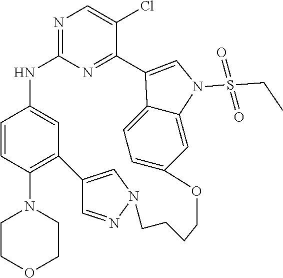 [SMILES: CCS(=O)(=O)n1cc2c3ccc(cc31)OCCCCn1cc(cn1)-c1cc(ccc1N1CCOCC1)Nc1ncc(Cl)c-2n1]

The synthesis method followed Embodiment 1, wherein Intermediate 11 in step 6 was replaced with 4-bromo-1-butanol.

¹H NMR (500 MHz, CDCl₃) δ 8.43-8.37 (m, 2H), 8.18 (d, J=8.8 Hz, 1H), 8.06 (s, 1H), 7.62-7.57 (m, 1H), 7.51-7.48 (m, 1H), 7.08 (d, J=8.5 Hz, 1H), 6.89 (dd, J=8.5, 2.7 Hz, 1H), 6.38 (dd, J=8.8, 2.3 Hz, 1H), 5.40-5.33 (m, 1H), 4.23 (t, J=6.3 Hz, 2H), 4.19 (t, J=5.8 Hz, 2H), 3.79 (t, J=4.4 Hz, 4H), 3.40 (q, J=7.4 Hz, 2H), 2.89 (t, J=4.5 Hz, 4H), 2.08-2.02 (m, 2H), 1.66 (dt, J=15.2, 6.8 Hz, 2H), 1.31 (t, J=6.3 Hz, 3H).

### Embodiment 22

### Preparation of (Z)-1¹-(ethylsulfonyl)-4⁶-Methoxy-7-methyl-4⁴-morpholino-1¹H,5¹H-9-oxa-3-aza-1(3,6)-indole-2(4,2)-pyrimidin-5(4,1)-pyrazol-4(1,3)-benzenacyclononaphane (WQX-1-80)

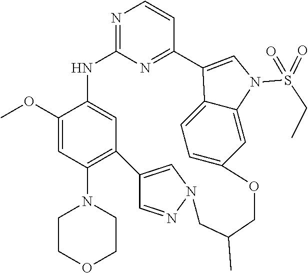 [SMILES: CCS(=O)(=O)n1cc2c3ccc(cc31)OCC(C)Cn1cc(cn1)-c1cc(c(OC)cc1N1CCOCC1)Nc1nccc-2n1]

The synthesis method referred to Embodiment 1. Wherein in Step 1 Intermediate 1 was replaced with 2,4-dichloropyrimidine, in step 4 Intermediate 6 was replaced with 1-bromo-2-fluoro-4-methoxy-5-nitrobenzene, and in step 6 Intermediate 11 was replaced with 3-bromo-2-methylpropanol.

¹H NMR (600 MHz, CDCl₃) δ 8.70 (s, 1H), 8.42 (d, J=5.1 Hz, 1H), 8.37 (d, J=8.9 Hz, 1H), 8.34 (s, 1H), 7.86 (s, 1H), 7.82 (s, 1H), 7.51 (d, J=2.2 Hz, 1H), 7.00 (d, J=5.0 Hz, 1H), 6.89 (s, 1H), 6.73 (s, 1H), 6.16 (dd, J=9.0, 2.3 Hz, 1H), 4.42-4.25 (m, 2H), 3.96 (s, 3H), 3.93-3.86 (m, 1H), 3.78 (m, 4H), 3.62 (m, 1H), 3.39 (q, J=7.3 Hz, 2H), 2.96 (m, 2H), 2.84 (m, 2H), 2.02 (m, 1H), 1.32 (t, J=7.4 Hz, 3H), 1.27 (d, 3H). MS (ESI): m/z 630 [M+H]⁺

### Embodiment 23

### Preparation of (Z)-1¹-(ethylsulfonyl)-4⁶-methoxy-8-methyl-4⁴-morpholino-1¹H,5¹H-9-oxa-3-aza-1(3,6)-indole-2(4,2)-pyrimidin-5(4,1)-pyrazol-4(1,3)-benzenacyclononaphane (WQX-1-86)

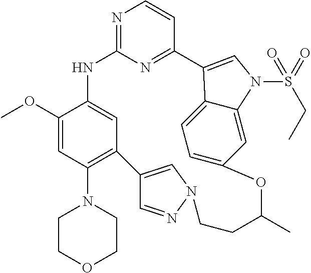 [SMILES: CCS(=O)(=O)n1cc2c3ccc(cc31)OC(C)CCn1cc(cn1)-c1cc(c(OC)cc1N1CCOCC1)Nc1nccc-2n1]

The synthesis method referred to Embodiment 1. Wherein in step 1, Intermediate 1 was replaced with 2,4-dichloropyrimidine, in step 4, Intermediate 6 was replaced with 1-bromo-2-fluoro-4-methoxy-5-nitrobenzene, and in step 6: Intermediate 11 was replaced with 4-bromobut-2-ol.

¹H NMR (600 MHz, CDCl₃) δ 8.71 (s, 1H), 8.41 (d, J=5.1 Hz, 1H), 8.33 (t, J=4.4 Hz, 2H), 7.86 (s, 1H), 7.82 (s, 1H), 7.48 (d, J=2.1 Hz, 1H), 7.00 (d, J=5.1 Hz, 1H), 6.92 (s, 1H), 6.73 (s, 1H), 6.10 (dd, J=9.2, 2.2 Hz, 1H), 4.79 (q, J=6.1 Hz, 1H), 4.04 (m, J=14.0, 6.7, 3.4 Hz, 1H), 3.96 (s, 3H), 3.91 (m, 1H), 3.85-3.75 (m, 4H), 3.39 (q, J=7.4 Hz, 2H), 3.02 (m, 2H), 2.80 (m, 2H), 2.03 (m, 2H), 1.52 (d, J=6.1 Hz, 3H), 1.31 (t, J=7.4 Hz, 3H). MS (ESI): m/z 630 [M+H]⁺

### Embodiment 24

### Preparation of (Z)-1′-(ethylsulfonyl)-6′-methoxy-4′-morpholino spiro[cyclopropane-7′-9-oxa-3-aza-1(3,6)-indole-2(4,2)-pyrimidin-5(4,1)-pyrazol-4(1,3)-benzenacyclononaphane] (WQX-1-87)

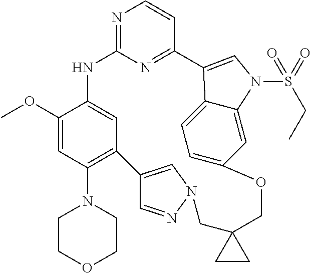 [SMILES: CCS(=O)(=O)n1cc2c3ccc(cc31)OCC1(CC1)Cn1cc(cn1)-c1cc(c(OC)cc1N1CCOCC1)Nc1nccc-2n1]

The synthesis method referred to Embodiment 1. Wherein in Step 1 Intermediate 1 was replaced with 2,4-dichloropyrimidine, in step 4 Intermediate 6 was replaced with 1-bromo-2-fluoro-4-methoxy-5-nitrobenzene, and in step 6 Intermediate 11 was replaced with (1-(bromomethyl)cyclopropyl) methanol.

¹H NMR (600 MHz, CDCl₃) δ 8.82 (s, 1H), 8.49 (d, J=0.7 Hz, 1H), 8.43 (d, J=5.0 Hz, 1H), 8.36 (d, J=8.9 Hz, 1H), 7.89 (s, 1H), 7.83 (s, 1H), 7.58 (d, J=2.2 Hz, 1H), 7.00 (d, J=5.1 Hz, 1H), 6.76 (d, J=0.7 Hz, 1H), 6.75 (s, 1H), 6.33 (dd, J=8.9, 2.3 Hz, 1H), 4.18 (s, 2H), 3.96 (s, 3H), 3.84-3.80 (m, 4H), 3.65 (s, 2H), 3.41 (q, J=7.4 Hz, 2H), 2.94-2.90 (m, 4H), 1.33 (t, J=7.3 Hz, 3H), 0.88 (d, 2H), 0.84 (d, J=4.7 Hz, 2H). MS (ESI): m/z 642 [M+H]⁺

### Embodiment 25

### Preparation of (Z)-1¹-(ethylsulfonyl)-4⁶-methoxy-4⁴-(morpholinomethyl)-1¹H,5¹H-9-oxa-3-aza-1(3,6)-indole-2(4,2)-pyrimidine-5(4,1)-pyrazole-4(1,3)-benzocyclononane (WQX-2-35)

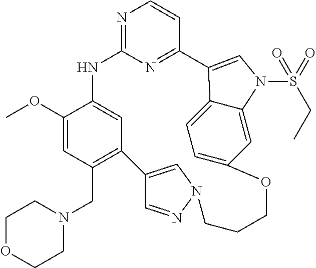 [SMILES: CCS(=O)(=O)n1cc2c3ccc(cc31)OCCCn1cc(cn1)-c1cc(c(OC)cc1CN1CCOCC1)Nc1nccc-2n1]

The synthesis method followed Embodiment 1. Wherein, in Step 1, Intermediate 1 was replaced with 2,4-dichloropyrimidine; in Step 4, Intermediate 6 was replaced with 1-bromo-2-fluoro-4-methoxy-5-nitrobenzene; and Intermediate 8 was replaced with Intermediate 21.

¹H NMR (600 MHz, CDCl₃) δ 8.83 (s, 1H), 8.44 (d, J=5.1 Hz, 1H), 8.31 (d, J=8.9 Hz, 1H), 8.03 (s, 2H), 7.80 (s, 1H), 7.49 (d, J=2.2 Hz, 1H), 7.02 (d, J=5.0 Hz, 1H), 7.00-6.95 (m, 1H), 6.91 (s, 1H), 6.13 (d, J=8.9 Hz, 1H), 4.50 (t, J=5.9 Hz, 2H), 3.98 (m, 5H), 3.73 (s, 4H), 3.43 (s, 2H), 3.39 (q, J=7.4 Hz, 2H), 2.50 (s, 4H), 2.44-2.36 (m, 2H), 1.33 (d, J=7.4 Hz, 3H). MS (ESI): m/z 630 [M+H]⁺

The preparation method of 4-(2-bromo-5-methoxy-4-nitrobenzyl) morpholine (Intermediate 21) was as follows:

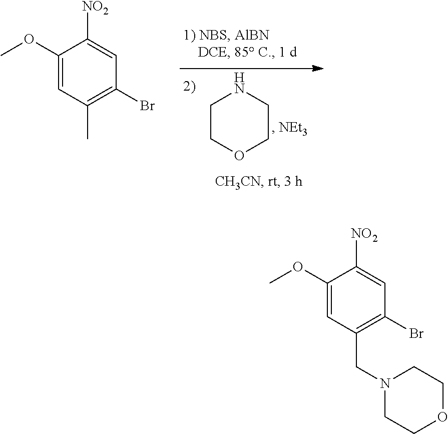 [SMILES: COc1cc(C)c(Br)cc1[N+](=O)[O-] ; C1COCCN1 ; COc1cc(CN2CCOCC2)c(Br)cc1[N+](=O)[O-]]

The oil bath was preheated to 85° C. 1.014 g of 1-bromo-4-methoxy-2-methyl-5-nitrobenzene, 0.8233 g of NBS, and 0.1479 g of AIBN were added to a 50 mL dried florence flask. The flask was purged with Ar gas three times, and under Ar gas protection, 15 mL of anhydrous DCE was added. The mixture was reacted at 85° C. for 1 day, then heating was stopped. After the mixture cooled naturally to room temperature, a saturated aqueous solution of sodium bicarbonate was added. The mixture was extracted with ethyl acetate, washed twice with saturated sodium bicarbonate solution and once with saturated sodium chloride solution. The organic layers were combined and dried with anhydrous sodium sulfate. After 2 hours, the organic layer was filtered and spin-dried. The product obtained was purged with Ar gas three times, and under Ar gas protection, the system was added with 15 mL of acetonitrile, 0.5 mL of morpholine, and 1.8 mL of triethylamine, and the mixture was stirred at room temperature. After 3 hours, the reaction progress was monitored by TLC (developing solvent was petroleum ether:ethyl acetate=5:1, v/v). The system was directly spin-dried, the product was purified by column chromatography (petroleum ether:ethyl acetate=3:1, v/v), to obtain Intermediate 21.

¹H NMR (500 MHz, CDCl₃) δ 8.06 (s, 1H), 7.39 (s, 1H), 3.97 (s, 3H), 3.78-3.74 (m, 4H), 3.59 (s, 2H), 2.55 (dd, J=5.6, 3.7 Hz, 4H).

### Embodiment 26

### Preparation of (Z)-1¹-(Ethylsulfonyl)-4⁶-methoxy-4⁴-(2-methylmorpholinyl)-1¹H,5¹H-9-oxa-3-aza-1(3,6)-indol-2(4,2)-pyrimidin-5(4,1)-pyrazol-4(1,3)-benzocyclononane (WQX-2-43)

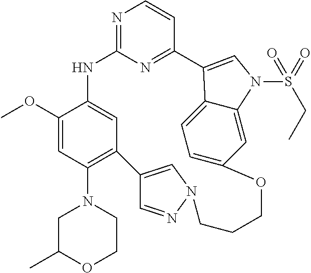 [SMILES: CCS(=O)(=O)n1cc2c3ccc(cc31)OCCCn1cc(cn1)-c1cc(c(OC)cc1N1CCOC(C)C1)Nc1nccc-2n1]

The synthesis method followed Embodiment 1. Wherein, in Step 1, Intermediate 1 was replaced with 2,4-dichloropyrimidine; in Step 4, Intermediate 6 was replaced with 1-bromo-2-fluoro-4-methoxy-5-nitrobenzene; and Intermediate 7 was replaced with 2-methylmorpholine.

¹H NMR (600 MHz, CDCl₃) δ 8.78 (s, 1H), 8.42 (d, J=5.1 Hz, 1H), 8.38 (d, J=0.7 Hz, 1H), 8.34 (d, J=8.9 Hz, 1H), 7.89 (s, 1H), 7.82 (s, 1H), 7.52 (d, J=2.2 Hz, 1H), 7.00 (d, J=5.0 Hz, 1H), 6.81 (d, J=0.7 Hz, 1H), 6.73 (s, 1H), 6.21 (dd, J=9.0, 2.3 Hz, 1H), 4.41 (m, J=5.9, 4.3 Hz, 2H), 3.97 (s, 3H), 3.94-3.85 (m, 3H), 3.82-3.76 (m, 2H), 3.40 (q, J=7.4 Hz, 2H), 2.97 (m, 2H), 2.77 (m, 1H), 2.51-2.42 (m, 3H), 1.33 (d, J=7.4 Hz, 3H), 1.16 (d, J=6.3 Hz, 3H). MS (ESI): m/z 630 [M+H]⁺.

### Embodiment 27

### Preparation of (Z)-1¹-(ethylsulfonyl)-2⁵-fluoro-4⁶-methoxy-4⁴-morpholinyl-1¹H,5¹H-9-oxa-3-aza-1(3,6)-indole-2(4,2)-pyrimidin-5(4,1)-pyrazol-4(1,3)-benzenacyclononaphane (WQX-2-79)

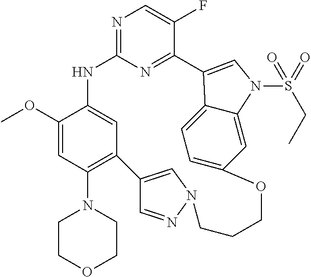 [SMILES: CCS(=O)(=O)n1cc2c3ccc(cc31)OCCCn1cc(cn1)-c1cc(c(OC)cc1N1CCOCC1)Nc1ncc(F)c-2n1]

The synthesis method followed Embodiment 1. Wherein in Step 1, Intermediate 1 was replaced with 2,4-dichloro-5-fluoropyrimidine, and in Step 4, Intermediate 6 is replaced with 1-bromo-2-fluoro-4-methoxy-5-nitrobenzene.

¹H NMR (600 MHz, CDCl₃) δ 8.66 (s, 1H), 8.40-8.38 (m, 2H), 8.35 (d, J=2.8 Hz, 1H), 8.07 (d, J=2.3 Hz, 1H), 7.85 (s, 1H), 7.55 (d, J=2.4 Hz, 1H), 6.88-6.85 (m, 1H), 6.74 (s, 1H), 6.16 (dd, J=9.0, 2.3 Hz, 1H), 4.42 (t, J=5.6 Hz, 2H), 3.97 (s, 3H), 3.94-3.90 (m, 2H), 3.80 (t, J=4.6 Hz, 4H), 3.41 (q, J=7.4 Hz, 2H), 2.94-2.90 (m, 4H), 2.45 (p, J=5.6 Hz, 2H), 1.33 (d, J=7.4 Hz, 3H). MS (ESI): m/z 634 [M+H]⁺.

### Embodiment 28

### Preparation of (E)-1¹-(ethylsulfonyl)-4⁶-methoxy-4⁴-morpholinyl-1¹H,5¹H-9-oxa-3-aza-1(3,6)-indole-2(4,2)-pyrimidine-5(3,1)-pyrazole-4(1,3)-benzocyclononaphthalene (FBH-4-6)

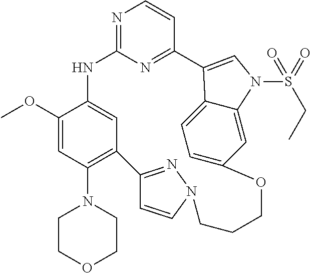 [SMILES: CCS(=O)(=O)n1cc2c3ccc(cc31)OCCCn1ccc(n1)-c1cc(c(OC)cc1N1CCOCC1)Nc1nccc-2n1]

The synthesis method followed Embodiment 1. Wherein, in Step 1, Intermediate 1 was replaced with 2,4-dichloropyrimidine; in Step 4, Intermediate 6 was replaced with 1-bromo-2-fluoro-4-methoxy-5-nitrobenzene; and in Step 5, Intermediate 9 was replaced with 1-(tetrahydro-2H-pyran-2-yl)-3-(4,4,5,5-tetramethyl-1,3,2-dioxaborolan-2-yl)-1H-pyrazole.

¹H NMR (600 MHz, DMSO-d₆) δ 8.45-8.42 (m, 2H), 8.27 (d, J=1.9 Hz, 1H), 7.92 (d, J=2.2 Hz, 1H), 7.62 (ddt, J=6.5, 2.9, 1.3 Hz, 1H), 7.57-7.54 (m, 1H), 7.39 (d, J=5.1 Hz, 1H), 7.28 (d, J=2.2 Hz, 1H), 6.81-6.77 (m, 2H), 5.79 (dd, J=9.1, 2.3 Hz, 1H), 4.32 (t, J=6.3 Hz, 2H), 3.97 (t, J=5.8 Hz, 2H), 3.94 (s, 3H), 3.67 (d, J=7.3 Hz, 2H), 3.62 (t, J=4.5 Hz, 4H), 2.83 (t, J=4.5 Hz, 4H), 2.21 (d, J=5.6 Hz, 2H), 1.12 (t, J=7.2 Hz, 3H). MS (ESI): m/z 615.23 [M+H]⁺.

### Embodiment 29

### Preparation of (Z)-4⁶-cyclopropoxy-1¹-(ethylsulfonyl)-4⁴-morpholinyl-1¹H,5¹H-9-oxa-3-aza-1(3,6)-indole-2(4,2)-pyrimidine-5(4,1)-pyrazole-4(1,3)-benzocyclononaphthalene (FBH-4-24)

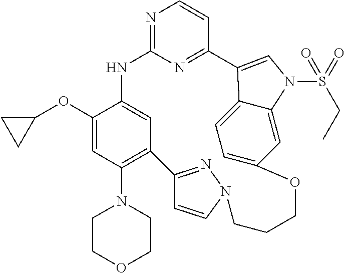 [SMILES: CCS(=O)(=O)n1cc2c3ccc(cc31)OCCCn1ccc(n1)-c1cc(c(OC3CC3)cc1N1CCOCC1)Nc1nccc-2n1]

The synthesis method followed Embodiment 1. Wherein, in Step 1, Intermediate 1 was replaced with 2,4-dichloropyrimidine, and in Step 4, Intermediate 6 was replaced with 1-bromo-2-fluoro-4-cyclopropoxy-5-nitrobenzene.

¹H NMR (600 MHz, CDCl₃) δ 8.78 (s, 1H), 8.41 (s, 1H), 8.33 (d, J=8.8 Hz, 1H), 7.82 (s, 1H), 7.74 (s, 1H), 7.70-7.63 (m, 2H), 7.52 (d, J=2.2 Hz, 1H), 7.10 (s, 1H), 6.99 (d, J=5.0 Hz, 1H), 6.81 (s, 1H), 6.20 (dd, J=8.9, 2.3 Hz, 1H), 4.42 (t, J=5.6 Hz, 2H), 3.94-3.88 (m, 2H), 3.81 (t, J=4.5 Hz, 4H), 3.40 (q, J=7.4 Hz, 2H), 2.99-2.85 (m, 4H), 2.52-2.39 (m, 2H), 1.32 (t, J=7.4 Hz, 3H), 0.90 (m, 2H), 0.87 (t, J=4.4 Hz, 2H). MS (ESI): m/z 641.24 [M+H]⁺.

### Embodiment 30

### Preparation of (Z)-1¹-(ethylsulfonyl)-4⁴-morpholinyl-4⁶-(trifluoromethoxy)-1¹H,5¹H-9-oxa-3-aza-1(3,6)-indole-2(4,2)-pyrimidine-5(4,1)-pyrazole-4(1,3)-benzocyclononaphthalene (FBH-4-48)

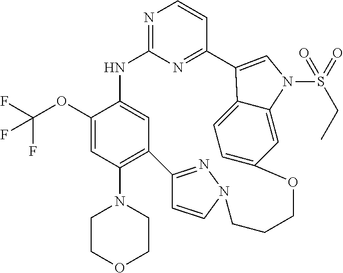 [SMILES: CCS(=O)(=O)n1cc2c3ccc(cc31)OCCCn1ccc(n1)-c1cc(c(OC(F)(F)F)cc1N1CCOCC1)Nc1nccc-2n1]

The synthesis method followed Embodiment 1. Wherein, in Step 1, Intermediate 1 was replaced with 2,4-dichloropyrimidine, and in Step 4, Intermediate 6 was replaced with 1-bromo-2-fluoro-4-trifluoromethoxy-5-nitrobenzene.

¹H NMR (600 MHz, CDCl₃) δ 8.91 (s, 1H), 8.46 (d, J=5.1 Hz, 1H), 8.42-8.38 (m, 1H), 8.26 (d, J=8.9 Hz, 1H), 7.84 (s, 1H), 7.68-7.66 (m, 1H), 7.53 (d, J=2.1 Hz, 1H), 7.46 (d, J=3.0 Hz, 1H), 7.07 (d, J=5.1 Hz, 1H), 7.03 (t, J=1.6 Hz, 1H), 6.87 (s, 1H), 4.43 (t, J=5.5 Hz, 2H), 3.94-3.88 (m, 2H), 3.79 (t, J=4.5 Hz, 4H), 3.41 (q, J=7.4 Hz, 2H), 2.88 (dd, J=5.7, 3.3 Hz, 4H), 2.49-2.44 (m, 2H), 1.33 (t, J=7.4 Hz, 3H). MS (ESI): m/z 669.20 [M+H]⁺.

### Embodiment 31

### Preparation of (Z)-1¹-(ethylsulfonyl)-4⁴-morpholinyl-4⁶-(trifluoromethyl)-1¹H,5¹H-9-oxa-3-aza-1(3,6)-indole-2(4,2)-pyrimidine-5(4,1)-pyrazole-4(1,3)-benzenacyclononaphane (FBH-3-92)

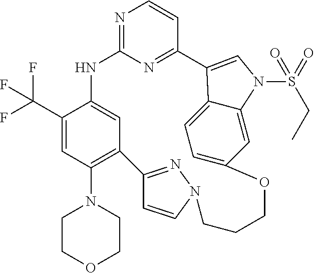 [SMILES: CCS(=O)(=O)n1cc2c3ccc(cc31)OCCCn1ccc(n1)-c1cc(c(C(F)(F)F)cc1N1CCOCC1)Nc1nccc-2n1]

The synthesis method followed Embodiment 1. Wherein, in Step 1, Intermediate 1 was replaced with 2,4-dichloropyrimidine, and in Step 4, Intermediate 6 was replaced with 1-bromo-2-fluoro-4-trifluoromethyl-5-nitrobenzene.

¹H NMR (600 MHz, DMSO-d₆) δ 8.71 (s, 1H), 8.48 (s, 1H), 8.44 (d, J=5.2 Hz, 1H), 8.26 (s, 1H), 8.13 (s, 1H), 8.07 (s, 1H), 7.60 (d, J=1.6 Hz, 1H), 7.56 (d, J=3.1 Hz, 1H), 7.37 (d, J=5.1 Hz, 1H), 7.25 (s, 1H), 7.14 (d, J=2.3 Hz, 1H), 4.32 (t, J=7.2 Hz, 2H), 3.75 (t, J=4.5 Hz, 4H), 3.61 (q, J=7.3 Hz, 2H), 2.90 (d, J=4.7 Hz, 4H), 2.08 (d, J=7.9 Hz, 2H), 1.23 (s, 2H), 1.06 (t, J=7.2 Hz, 3H). MS (ESI): m/z 653.20 [M+H]⁺.

### Embodiment 32

### Preparation of (Z)-1¹-(ethylsulfonyl)-4⁶-isopropoxy-4⁴-morpholinyl-1¹H,5¹H-9-oxa-3-aza-1(3,6)-indole-2(4,2)-pyrimidin-5(4,1)-pyrazol-4(1,3)-benzocyclononaphthalene (FBH-3-84)

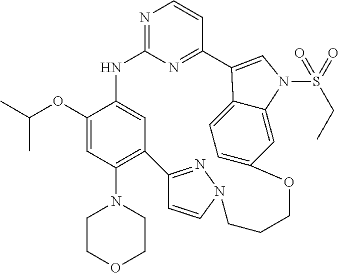 [SMILES: CCS(=O)(=O)n1cc2c3ccc(cc31)OCCCn1ccc(n1)-c1cc(c(OC(C)C)cc1N1CCOCC1)Nc1nccc-2n1]

The synthesis method followed Embodiment 1. Wherein, in Step 1, Intermediate 1 was replaced with 2,4-dichloropyrimidine, and in Step 4, Intermediate 6 was replaced with 1-bromo-2-fluoro-4-isopropoxy-5-nitrobenzene.

¹H NMR (600 MHz, DMSO-d₆) δ 8.45 (d, J=5.1 Hz, 1H), 8.32 (d, J=1.8 Hz, 2H), 8.30 (d, J=8.9 Hz, 1H), 8.00 (d, J=0.7 Hz, 1H), 7.81 (s, 1H), 7.76 (s, 1H), 7.44 (d, J=5.1 Hz, 1H), 7.27 (d, J=2.3 Hz, 1H), 6.82 (s, 1H), 4.71 (p, J=6.0 Hz, 1H), 4.36 (t, J=6.5 Hz, 2H), 4.07 (dd, J=11.3, 6.3 Hz, 2H), 3.68 (dd, J=6.0, 3.3 Hz, 5H), 2.82 (t, J=4.6 Hz, 4H), 2.17 (q, J=9.4, 8.4 Hz, 2H), 2.04-1.94 (m, 2H), 1.34 (d, J=6.1 Hz, 6H), 1.12 (t, J=7.3 Hz, 3H). MS (ESI): m/z 643.26 [M+H]⁺.

### Embodiment 33

### Preparation of (Z)-4⁶-cyclopropyl-1¹-(ethylsulfonyl)-4⁴-morpholino-1¹H,5¹H-9-oxa-3-aza-1(3,6)-indole-2(4,2)-pyrimidine-5(4,1)-pyrazol-4(1,3)-benzenacyclononaphane (FBH-3-66)

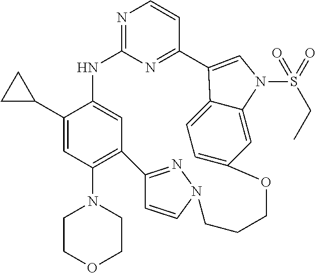 [SMILES: CCS(=O)(=O)n1cc2c3ccc(cc31)OCCCn1ccc(n1)-c1cc(c(C3CC3)cc1N1CCOCC1)Nc1nccc-2n1]

The synthesis method followed Embodiment 1. Wherein, in Step 1, Intermediate 1 was replaced with 2,4-dichloropyrimidine, and in Step 4: Intermediate 6 was replaced with 1-bromo-2-fluoro-4-cyclopropyl-5-nitrobenzene.

¹H NMR (600 MHz, DMF-d₇) δ 8.55 (s, 1H), 8.42 (d, J=5.1 Hz, 1H), 8.33-8.29 (m, 1H), 8.27 (s, 1H), 8.00 (s, 1H), 7.93 (d, J=0.7 Hz, 1H), 7.60 (d, J=1.5 Hz, 1H), 7.57-7.56 (m, 1H), 7.35 (d, J=5.1 Hz, 1H), 7.17 (d, J=2.3 Hz, 1H), 6.68 (s, 1H), 5.43 (dd, J=9.2, 2.4 Hz, 1H), 4.33 (t, J=7.0 Hz, 2H), 4.23-4.15 (m, 2H), 3.70 (t, J=4.5 Hz, 4H), 3.63 (q, J=7.3 Hz, 2H), 2.82 (t, J=4.4 Hz, 4H), 2.19-2.06 (m, 3H), 1.23 (s, 2H), 0.95-0.90 (m, 2H), 0.61-0.57 (m, 2H). MS (ESI): m/z 625.25 [M+H]⁺.

### Embodiment 34

### Preparation of (Z)-1¹-(ethylsulfonyl)-4⁶-isopropyl-4⁴-morpholino-1¹H,5¹H-9-oxa-3-aza-1(3,6)-indole-2(4,2)-pyrimidine-5(4,1)-pyrazole-4(1,3)-benzenacyclononaphane (FBH-3-48)

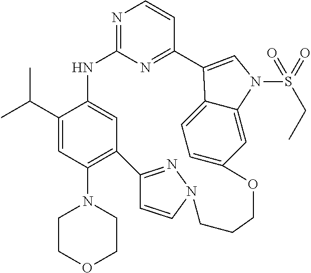 [SMILES: CCS(=O)(=O)n1cc2c3ccc(cc31)OCCCn1ccc(n1)-c1cc(c(C(C)C)cc1N1CCOCC1)Nc1nccc-2n1]

The synthesis method followed Embodiment 1. Wherein, in Step 1, Intermediate 1 was replaced with 2,4-dichloropyrimidine, and in Step 4, Intermediate 6 was replaced with 1-bromo-2-fluoro-4-isopropyl-5-nitrobenzene.

¹H NMR (600 MHz, DMSO-d₆) δ 8.69 (s, 1H), 8.56 (s, 1H), 8.38 (d, J=5.1 Hz, 1H), 8.20 (d, J=2.6 Hz, 1H), 7.91 (d, J=0.7 Hz, 1H), 7.79 (s, 1H), 7.64-7.61 (m, 2H), 7.58-7.54 (m, 1H), 7.25 (d, J=5.1 Hz, 1H), 7.12 (d, J=2.3 Hz, 1H), 6.92 (s, 1H), 4.36-4.24 (m, 4H), 3.72 (t, J=4.5 Hz, 4H), 3.60 (q, J=7.2 Hz, 2H), 2.86 (t, J=4.5 Hz, 4H), 2.07 (d, J=8.1 Hz, 2H), 1.24 (t, J=5.5 Hz, 3H), 1.12 (d, J=6.8 Hz, 6H). MS (ESI): m/z 627.26 [M+H]⁺.

### Embodiment 35

### Preparation of (Z)-4⁶-ethyl-1¹-(ethylsulfonyl)-4⁴-morpholino-1¹H,5¹H-9-oxa-3-aza-1(3,6)-indole-2(4,2)-pyrimidine-5(4,1)-pyrazol-4(1,3)-benzenacyclononaphane (FBH-3-47)

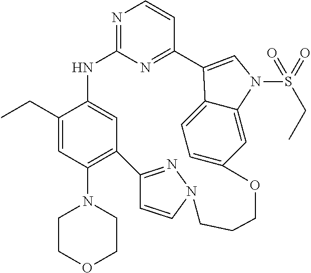 [SMILES: CCc1cc(N2CCOCC2)c2cc1Nc1nccc(n1)-c1cn(S(=O)(=O)CC)c3cc(ccc13)OCCCn1ccc-2n1]

The synthesis method followed Embodiment 1. Wherein, in Step 1, Intermediate 1 was replaced with 2,4-dichloropyrimidine, and in Step 4, Intermediate 6 was replaced with 1-bromo-2-fluoro-4-ethyl-5-nitrobenzene.

¹H NMR (600 MHz, DMSO-d₆) δ 8.64 (s, 1H), 8.51 (s, 1H), 8.39 (d, J=5.1 Hz, 1H), 8.23 (s, 1H), 7.90 (d, J=0.7 Hz, 1H), 7.84 (s, 1H), 7.63 (dtd, J=8.4, 2.9, 1.4 Hz, 1H), 7.58-7.53 (m, 1H), 7.28 (d, J=5.1 Hz, 1H), 7.13 (d, J=2.3 Hz, 1H), 6.90 (s, 1H), 4.29 (m, 4H), 3.72 (t, J=4.5 Hz, 4H), 3.61 (q, J=7.3 Hz, 2H), 2.85 (d, J=5.2 Hz, 4H), 2.71 (q, J=7.5 Hz, 2H), 2.07 (d, J=8.1 Hz, 2H), 1.05 (m, 6H). MS (ESI): m/z 613.25 [M+H]⁺.

### Embodiment 36 Activity Inhibition Experiments of Cyclic 2-Aminopyrimidine Compounds Against Wild-Type EGFR and Mutant EGFR Kinase

EGFR (WT) is a wild-type epidermal growth factor receptor. EGFR^{(19del/T790M/C797S)} is a triple mutant epidermal growth factor receptor with partial deletion of exon 19, mutation of amino acid residue 790 from threonine to methionine, and mutation of amino acid residue 797 from cysteine to serine.

Enzyme-Linked Immunosorbent Assay (ELISA) was used to detect the inhibitory effect of compounds on kinase activity. EGFRWT was purchased from Eurofins company, and EGFR^{19del/T790M/C797S} kinase was purchased from BPS Bioscience company.

The main experimental steps were as follows: Enzyme reaction substrate Poly (Glu, Tyr)_{4:1} was diluted with potassium free PBS (10 mM sodium phosphate buffer, 150 mM NaCl, pH 7.2-7.4) to 20 μg/mL, and reacted at 37° C. for 12 hours to 16 hours to coat the enzyme-linked immunosorbent assay plate. ATP solution (final concentration 5 μM) diluted with reaction buffer (50 mM HEPES pH 7.4, 50 mM MgCl₂, 0.5 mM MnCl₂, 0.2 mM Na₃VO₄, 1 mM DTT) was added to each well, followed by the test compound or solvent control, then the kinase reaction was initiated and the plate was shaken at 37° C. for 1 hour. The plate was washed three times with T-PBS, then added antibody PY99. The plate was shaken at 37° C. for 0.5 hours. After washing the plate with T-PBS, added horseradish peroxidase-labeled IgG and shaken at 37° C. for 0.5 hours. After washing the plate again, added 2 mg/mL of OPD colorimetric solution and incubated at 25° C. in the dark for 1 minutes to 10 minutes. 2 M H₂SO₄ was added to terminate the reaction, and an adjustable wavelength microplate reader SPECTRA MAX 190 was used to detect the reaction at 492 nm. The IC₅₀ values were obtained from the inhibition curve analysis.

The compound numbers and the detection results of the inhibitory activity of the compounds against various kinase were listed in Table 1 (corresponding to the compound numbers in the above embodiments).

TABLE 1
Test results of inhibitory activity of cyclic 2-aminopyrimidine compounds against kinases

| | | | The average IC₅₀ value (nM) for inhibiting the |
|---|---|---|---|
| NO. Of the | | | activity of different tyrosine kinases |
| compounds | EGFR^{WT} | EGFR^{L858R/T790M/C797S} | EGFR^{19del/T790M/C797S} |
| HC871 | >1000 | 4.9 | 6.3 |
| HC8990 | 342.2 | 1 | 3.4 |
| HC8991 | >1000 | / | 6.8 |
| HC9001 | >1000 | 183.3 | 82.3 |
| HC9002 | >1000 | 59.0 | 24.7 |
| HC9003 | >1000 | 69.7 | 29.0 |
| HC90032 | >1000 | 342.7 | 191.9 |
| HC9004 | >1000 | 383.5 | 195.1 |
| HC90042 | >1000 | >1000 | >1000 |
| HC916 | >1000 | 167.5 | 128.7 |
| HC9281 | >1000 | 30.5 | 30.5 |
| HC9282 | >1000 | 45.8 | 45.8 |
| HC9284 | 153.5 | 35.1 | 35.1 |
| HC9452 | 44.7 | 4.3 | 4.3 |
| HC950 | 111.5 | 0.4 | 4.3 |
| HC950M | 37.6 | 1.9 | 7.3 |
| HC9591 | 632.1 | 2.0 | 1.7 |
| HC9592 | 898.2 | 1.4 | 2.9 |
| HC9594 | 112.4 | 2.2 | 1.7 |
| HC9596 | 186.2 | 11.8 | 7.4 |
| HC8714 | 802.2 | 9.3 | 2.7 |
| WQX-1-80 | >1000 | 53.2 | 23.3 |
| WQX-1-86 | >1000 | 12.6 | 8.2 |
| WQX-1-87 | >1000 | 63.3 | 33.0 |
| WQX-2-35 | 140.6 | 14.1 | 1 |
| WQX-2-43 | 146.1 | 57.8 | / |
| WQX-2-79 | 254.9 | 102.4 | / |
| FBH-4-6 | >1000 | 146.5 | / |
| FBH-4-24 | 188.6 | 98.5 | / |
| FBH-4-48 | >1000 | 134.7 | / |
| FBH-3-92 | >1000 | 159.1 | / |
| FBH-3-84 | >1000 | / | 266.6 |
| FBH-3-66 | >1000 | / | 33.0 |
| FBH-3-48 | >1000 | / | 8.2 |
| FBH-3-47 | >1000 | 54.8 | 23.3 |
| Brigatinib | 209.8 | 6.0 | 24.3 |
| Osimertinib | 190.9 | 207.3 | 271.1 |
| 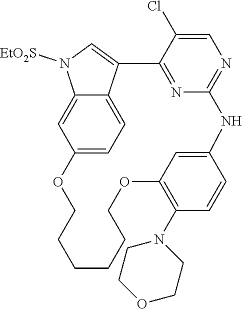 | 189.8 | 14.4 | 9.0 |

Note:
“/” in the table indicates that the data was not tested.

As shown in Table 1, the cyclic 2-aminopyrimidine compounds of the present disclosure effectively and selectively inhibited the activity of EGFR protein kinase resistant mutants (EGFRL858R/T790M/C797S and EGFR19del/T790M/C797S), overcoming the clinical resistance of non-small cell lung cancer patients induced by existing third-generation selective EGFR^{T790M} small molecule inhibitors such as Osimertinib (AZD9291), Olmutinib (HM6171), Rociletinib (CO-1686), etc.

### Embodiment 37 Experiment of the Compound Inhibition on EGFR Mutant Cell Proliferation

Ba/F3 cells were obtained from Deutsche Sammlung von Mikroorganismen und Zellkulturen GmbH (DSMZ). Ba/F3 cells stably expressing EGFR19del/T790M/C797S or EGFR L858R/T790M/C797S were generated by infecting parental cells with corresponding retroviruses. PC-9-OR was a tumor cell model obtained by simultaneously knocking in T790M and C797S mutations into 19del-activating mutation-dependent PC-9 tumor cells using CRISPR technology. This model, which proliferated dependent on EGFR19del/T790M/C797S resistance mutation, was used to evaluate the inhibitory activity of compounds against EGFR19del/T790M/C797S resistance mutants (Cancer Science. 2022; 113:709-720). All cells used in the study were verified by short tandem repeat (STR) analysis conducted by Genesky.

Tumor cell viability was assessed using a CCK-8 cell counting kit (A311-01/02, Vazyme, Nanjing, China). Cells were seeded at a density of 3000 cells per well into 96-well plates and cultured overnight. Subsequently, the cells were treated with compounds of different concentrations and incubated for 72 hours. After 72 hours, 10 μL of CCK-8 solution was added to each well. The plates were then returned to the incubator and incubated at 37° C. for 2 hours. The absorbance was measured at 450 nm using a microplate reader. A calibration curve was constructed using data obtained from wells containing known numbers of viable cells.

The test results were presented in Table 2: the cyclic 2-aminopyrimidine compounds of the present disclosure effectively and selectively inhibited the growth and proliferation of EGFR mutant cells.

TABLE 2
Inhibitory of compounds on the proliferation
activity of EGFR-mutant cells

| | Average value of IC₅₀ of inhibition | | | |
|---|---|---|---|---|
| | against EGFR-mutant cells growth (μM) | | | |
| | | BaF3-EGFR | BaF3-EGFR | PC-9-OR |
| | BaF3 | (19del/ | (L858R/ | (19del/ |
| NO. of the | parent | T790M/ | T790M/ | T790M/ |
| compound | cell | C797S) | C797S) | C797S) |
| HC871 | 0.217 | 0.070 | 0.074 | 0.085 |
| HC8990 | 3.289 | 0.429 | 0.325 | 0.531 |
| HC9452 | 1.794 | 0.472 | 0.591 | 0.081 |
| HC950 | 0.95 | 0.170 | 0.336 | 0.149 |
| HC950M | 0.445 | 0.086 | 0.108 | 0.149 |
| HC9591 | 1.815 | 0.189 | 0.301 | 0.269 |
| HC9594 | >10 | 1.223 | 0.735 | 0.925 |
| HC9596 | 3.185 | 0.371 | 0.455 | / |
| HC8714 | 0.274 | 0.091 | 0.075 | 0.155 |
| WQX-1-80 | 1.872 | 0.311 | 0.734 | / |
| WQX-1-86 | 1.995 | 0.465 | / | / |
| FBH-3-47 | 2.233 | 0.229 | 0.327 | / |
| Brigatinib | 1.476 | 0.221 | 0.777 | 1.454 |
| Osimertinib | 2.370 | 2.092 | 2.304 | 2.103 |

Note:
“/” in the table indicated that the data was not tested.

### Embodiment 38 Experiment of Inhibition of the Compound Against EGFR Phosphorylation Level in Tumor Cells

After overnight starvation, cells were treated with the test compound for 2 hours, followed by stimulation with 50 ng/mL EGF for 15 minutes. Subsequently, cells were collected with trypsin and then lysed with SDS lysis buffer, followed by centrifugation at 12000 g for 30 minutes. Protein concentration was measured using a BCA protein assay kit (Catalog No. 23227, Thermo Fisher Scientific). After heated at 100° C. for 15 minutes, protein samples were separated on an 8% to 12% SDS-PAGE gel and then transferred to a nitrocellulose membrane (Life Technologies). The membrane was blocked in 5% skim milk in TBST solution and incubated overnight at 4° C. with primary antibodies against phosphorylated EGFR (Tyr1068; dilution, 1:500; catalog number 2234L, CST) and β-actin (dilution, 1:20000; catalog number 60008-1-Ig, Proteintech), followed by incubation with secondary antibodies (catalog number 111-035-003, Jackson) for one hour.

The experimental results were shown in FIGURE: The cyclic 2-aminopyrimidine compounds of the present disclosure effectively inhibited the EGFR phosphorylation levels in EGFR19del/T790M/C797S mutant cells and EGFR L858R/T790M/C797S mutant cells.

### Embodiment 39 Experiment to Determine Pharmacokinetic Parameters of the Compounds in Rats

Sprague-Dawley rats (males, 3 per group), weighing 180 g to 220 g, were administered test compounds intravenously and orally at doses of 1 mg/kg and 5 mg/kg, respectively. The compounds were dissolved in a mixed solvent (5% DMSO+10% Solutol+85% PBS) for intravenous injection. 0.5% CMC-Na solution (800 cps to 1200 cps) was used as a suspension agent for oral administration. At designated time points, 0.2 mL of blood samples were collected via the jugular vein and placed into tubes containing K2-EDTA, stored on ice, and then centrifuged. Sampling intervals for the injection group were 0.083, 0.25, 0.5, 1, 2, 4, 8, and 24 hours, while for the oral group, sampling intervals were 0.25, 0.5, 1, 2, 4, 6, 8, and 24 hours. The blood samples were centrifuged at 6800 g for 6 minutes at 2° C. to 8° C. within 1 hour after collection and stored frozen at approximately −80° C. Subsequently, 200 μL of MeOH was used to precipitate 20 μL of plasma samples containing 100 ng/mL IS. The mixture was vortexed for 1 minute and then centrifuged at 18000 g for 7 minutes. 200 μL of the supernatant was transferred to a 96-well plate, and 1 μL of the supernatant sample was injected for LC-MS/MS analysis. Finally, Phoenix WinNonlin7.0 software was used to calculate the pharmacokinetic (PK) parameters.

TABLE 3
Pharmacokinetic parameters of the compounds in rats

| | Route and | | | | |
|---|---|---|---|---|---|
| | dosage of | | | CL(mL/ | |
| Compound | administration | T_{1/2} (h) | Cₘₐₓ (ng/mL) | min/kg) | F (%) |
| 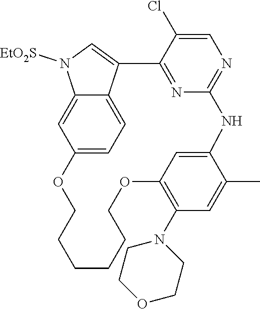 | IV(1 mg/kg) PO(5 mg/kg) | 1.35 ± 0.18 2.57 ± 2.01 | 2422.9 ± 165.1 134.6 ± 10.7 | 16.2 ± 1.4 \ | \ 11.9 ± 1.8 |
| HC9591 | IV(1 mg/kg | 7.10 ± 0.95 | 1572.9 ± 219.5 | 9.2 ± 2.1 | \ |
| | PO(5 mg/kg) | 8.13 ± 0.08 | 329.6 ± 56.5 | \ | 39.6 ± 9.0 |

As shown in Table 3, the pharmacokinetic properties of the cyclic 2-aminopyrimidine compounds of the present disclosure in rats were significantly superior to those reported in CN116102575A. The compounds of the present disclosure exhibited higher oral bioavailability (F %=39.6%), lower clearance (CL=9.2 mL/min/kg), and a longer half-life (T½=7.10 h).

The embodiments above merely express several implementations of the present disclosure. The descriptions of the embodiments are relatively specific and detailed, but may not therefore be construed as the limitation on the patent scope of the present disclosure. It should be noted that a person of ordinary skill in the art may further make several variations and improvements without departing from the concept of the present disclosure. These variations and improvements all fall within the protection scope of the present disclosure. Therefore, the patent protection scope of the present disclosure shall be defined by the appended claims.

What is claimed is:

1. A Cyclic 2-aminopyrimidine compound, or its pharmaceutically acceptable salt, stereoisomer, or prodrug molecule, having a structure as shown in formula (I): 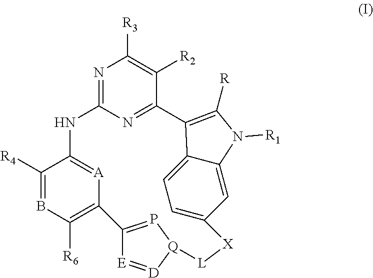
wherein A is selected from: CR₅, N;
B is selected from: CR₇, N;
D. E, Q, and P are independently selected from CR₈ and N, respectively;
X is selected from: —O—, —O(═O)C—, —S—, —S(═O)₂—, —S(═O)—, —C(═O)O—, —C(═O)NH—, —CH₂—, —C═C—, —C≡C—, —NH—, —NH(C═O)—;
R₁ is selected from: H, hydroxyl, C₆˜C₁₀ aryl, 5-10-membered heteroaryl, —C(═O)R₉, —S(═O)₂R₉, —S(═O)R₉, —P(═O)(R₉)₂, R₁₀ substituted or unsubstituted C₁˜C₈ alkyl, R₁₀ substituted or unsubstituted C₃˜C₈ cycloalkyl, R₁₀ substituted or unsubstituted 3-8-membered heterocyclic group; each R₉ is independently selected from: R₁₀ substituted or unsubstituted C₁˜C₈ alkyl, R₁₀ substituted or unsubstituted C₁˜C₈ alkoxy, R₁₀ substituted or unsubstituted C₃˜C₈ cycloalkyl, R₁₀ substituted or unsubstituted C₃˜C₈ cycloalkoxy, amino group, C₁˜C₈ alkylamino, (C₁˜C₈ alkyl)₂ amino; each R₁₀ is independently selected from: H, deuterium, C₁˜C₈ alkyl, C₃˜C₈ cycloalkyl, C₁˜C₈ alkoxy, C₁˜C₈ alkylthio, C₁˜C₈ alkylamino, 3-8-membered heterocyclic group, halogen, hydroxyl, cyano, amino group, —C(═O)R₁₁, —S(═O)₂R₁₁, —S(═O)R₁₁, C₆˜C₁₀ aryl, 5-10-membered heteroaryl; each R₁₁ is independently selected from: C₁˜C₈ alkyl, halogen substituted C₁˜C₈ alkyl, C₁˜C₈ alkoxy, C₂˜C₈ alkenyl;
R₂, R₃, R₄, R₅, R₇, R₈ are independently selected from H, halogen, cyano, nitro, amino group, 5-10-membered heteroaryl, C₆˜C₁₀ aryl, R₁₀ substituted or unsubstituted C₁˜C₈ alkyl, R₁₀ substituted or unsubstituted C₂˜C₈ alkenyl, R₁₀ substituted or unsubstituted C₃˜C₈ cycloalkyl, R₁₀ substituted or unsubstituted C₁˜C₈ alkoxy, R₁₀ substituted or unsubstituted C₃˜C₈ cycloalkoxy, —NR₁₂C(═O)R₁₁, —C(═O)R₁₁, —C(═O)N(R₁₂)₂; each R₁₂ is independently selected from: H, C₁˜C₈ alkyl;
or, R₂, R₃, together with the attached carbon atoms form: a R₁₀ substituted or unsubstituted benzene ring, R₁₀ substituted or unsubstituted C₃˜C₈ cycloalkyl, R₁₀ substituted or unsubstituted 5-8-membered heterocyclic ring, R₁₀ substituted or unsubstituted 5-10-membered heteroaromatic ring;
or, R₄ and R₇, together with the attached carbon atoms form: R₁₀ substituted or unsubstituted benzene ring, R₁₀ substituted or unsubstituted C₃-C₈ cycloalky, R₁₀ substituted or unsubstituted 5-8-membered heterocyclic ring, R₁₀ substituted or unsubstituted 5-10-membered heteroaromatic ring;
R₆ is selected from: —NR₁₃R₁₄, —CR₁₃R₁₄R₁₅, —(CH₂)ₙR₁₃R₁₄R₁₅, —(CH₂)ₙNR₁₃R₁₄; each R₁₃ and each R₁₄ is independently selected from: H, R₁₀ substituted or unsubstituted C₁˜C₈ alkyl, or R₁₃, R₁₄ together with the attached N or C form a substituted or unsubstituted 3 to 12 membered heterocyclic group; R₁₅ is selected from: H, C₁˜C₈ alkyl; N is selected from: 1, 2, 3;
R is selected from H, C₁˜C₈ alkyl, —C(═O)R₁₁, —C(═O)N(R₁₂)₂, or R together with the attached carbon form —C(═O);
L is selected from R₁₆ substituted or unsubstituted C₁˜C₈ alkylene, R₁₆ substituted or unsubstituted C₂˜C₈ alkenediyl;
R₁₆ is selected from: H, deuterium, C₁˜C₈ alkyl, halogen substituted C₁˜C₈ alkyl, C₃˜C₈ cycloalkyl, amino, halogen, hydroxyl, cyano, C₁˜C₈ alkoxy, C₁˜C₈ alkylthio, C₁˜C₈ alkylamino, 3-8-membered heterocyclic group; alternatively, two R₁₆ connected to the same carbon atom or two R₁₆ connected to adjacent carbon atoms to form a C₃˜C₈ cyclic alkyl group.

2. The cyclic 2-aminopyrimidine compound or its pharmaceutically acceptable salt or stereoisomer or prodrug molecule according to claim 1, wherein the cyclic 2-aminopyrimidine compound has the structure as shown in formula (II)

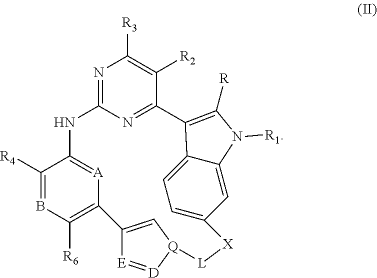

3. The cyclic 2-aminopyrimidine compound or its pharmaceutically acceptable salt or stereoisomer or prodrug molecule according to claim 1, wherein A is selected from CH, N.

4. The cyclic 2-aminopyrimidine compound or its pharmaceutically acceptable salt or stereoisomer or prodrug molecule according to claim 1, wherein B is selected from CR₇, N; R₇ is selected from: H, C₅˜C₆ cycloalkyl, C₂˜C₆ alkenyl, and C₁˜C₅ alkyl.

5. The cyclic 2-aminopyrimidine compound or its pharmaceutically acceptable salt or stereoisomer or prodrug molecule according to claim 4, wherein B is selected from CR₇, N; R₇ is selected from: H, cyclopentyl, methyl vinyl, 2-methylpropenyl, isobutyl, 3,3-dimethylbutenyl, 3,3-dimethylbutyl.

6. The cyclic 2-aminopyrimidine compound or its pharmaceutically acceptable salt or stereoisomer or prodrug molecule according to claim 2, wherein E is CH; D is CH or N; Q is N.

7. The cyclic 2-aminopyrimidine compound or its pharmaceutically acceptable salt or stereoisomer or prodrug molecule according to claim 1, wherein E is CH; D is CH or N; Q is N; P is CH or N.

8. The cyclic 2-aminopyrimidine compound or its pharmaceutically acceptable salt or stereoisomer or prodrug molecule according to claim 1, wherein R₁ is selected from: H, —C(═O)R₉, —S(═O)₂R₉, —S(═O)R₉, —P(═O)(R₉)₂, R₁₀ substituted or unsubstituted C₁˜C₄ alkyl; each R₉ is independently selected from: C₁˜C₄ alkyl, C₁˜C₄ alkoxy, amino, C₁˜C₄ alkylamino, (C₁˜C₄ alkyl)₂-amino, halogen substituted C₁˜C₄ alkyl; R₁₀ in R₁ is selected from H, deuterium, C₁˜C₄ alkyl, C₁˜C₄ alkoxy, halogen, hydroxyl, cyano, and amino.

9. The cyclic 2-aminopyrimidine compound or its pharmaceutically acceptable salt or stereoisomer or prodrug molecule according to claim 8, wherein R₁ is selected from: H, ethanesulfonyl, methylsulfonyl, propylsulfonyl, methyl, ethyl, propyl, acetyl, trifluoroacetyl, aminoacyl, methylaminoacyl, dimethylaminoacyl, aminosulfonyl, methylaminosulfonyl, dimethylaminosulfonyl, —P(═O)(OCH₃)₂, —P(═O)(CH₃)₂.

10. The cyclic 2-aminopyrimidine compound or its pharmaceutically acceptable salt or stereoisomer or prodrug molecule according to claim 1, wherein R₂ and R₃ are independently selected from: H, halogen, cyano, nitro, amino, R₁₀ substituted or unsubstituted C₁˜C₄ alkyl, R₁₀ substituted or unsubstituted C₂˜C₄ alkenyl, R₁₀ substituted or unsubstituted C₃˜C₆ cycloalkyl, R₁₀ substituted or unsubstituted C₁˜C₄ alkoxy, R₁₀ substituted or unsubstituted C₃˜C₆ cycloalkoxy, —NR₁₂C(═O)R₁₁, —C(═O)R₁₁, —C(═O)N(R₁₂)₂;
each R₁₀ in R₂ and R₃ is independently selected from H, deuterium, C₁˜C₄ alkyl, C₁˜C₄ alkoxy, halogen, hydroxyl, cyano, and amino;
each R₁₁ in R₂ and R₃ is independently selected from: C₁˜C₄ alkyl, halogen substituted C₁˜C₄ alkyl, C₁˜C₄ alkoxy, C₂˜C₄ alkenyl;
each R₁₂ in R₂ and R₃ is independently selected from: H, C₁˜C₄ alkyl;
or, R₂, R₃, together with the attached carbon atoms form R₁₀ substituted or unsubstituted benzene ring, R₁₀ substituted or unsubstituted C₅˜C₆ cycloalkyl, R₁₀ substituted or unsubstituted 5-6-membered heterocyclic ring, or R₁₀ substituted or unsubstituted 5-6 membered heteroaromatic ring.

11. The cyclic 2-aminopyrimidine compound or its pharmaceutically acceptable salt or stereoisomer or prodrug molecule according to claim 10, wherein R₂ is selected from —H, chlorine, bromine, fluorine, iodine, methyl, methoxy, methoxycarbonyl, ethoxycarbonyl, isopropoxycarbonyl, nitro, amino, trifluoromethyl, acetamido-, acrylamido-, isobutyramido-, propionamido-, and cyano; R₃ is H;
or, R₂, R₃, together with the attached carbon atoms form: benzene ring, pyrrole ring, thiophene ring, pyrazole ring, pyridine ring, furan ring.

12. The cyclic 2-aminopyrimidine compound or its pharmaceutically acceptable salt or stereoisomer or prodrug molecule according to claim 1, wherein R₂ is not H when R₄ is selected from H or methyl.

13. The cyclic 2-aminopyrimidine compound or its pharmaceutically acceptable salt or stereoisomer or prodrug molecule according to claim 1, wherein R₄ is selected from: H, halogen, cyano, nitro, amino, R₁₀ substituted or unsubstituted C₁˜C₄ alkyl, R₁₀ substituted or unsubstituted C₂˜C₄ alkenyl, R₁₀ substituted or unsubstituted C₃˜C₆ cycloalkyl, R₁₀ substituted or unsubstituted C₁˜C₄ alkoxy, R₁₀ substituted or unsubstituted C₃˜C₆ cycloalkoxy, —NR₁₂C(═O)R₁₁, —C(═O)R₁₁, —C(═O)N(R₁₂)₂;
each R₁₀ in R₄ is independently selected from: H, deuterium, C₁˜C₄ alkyl, C₁˜C₄ alkoxy, halogen, hydroxyl, cyano, and amino;
each R₁₁ in R₄ is independently selected from: C₁˜C₄ alkyl, halogen substituted C₁˜C₄ alkyl, C₁˜C₄ alkoxy, C₂˜C₄ alkenyl;
each R₁₂ in R₄ is independently selected from: H and C₁˜C₄ alkyl.

14. The cyclic 2-aminopyrimidine compound or its pharmaceutically acceptable salt or stereoisomer or prodrug molecule according to claim 13, wherein R₄ is selected from: H, methyl, ethyl, isopropyl, methoxy, ethoxy, isopropoxy, n-propoxy, isobutyloxy, deuterated methoxy, trifluoromethoxy, cyclopropoxy, trifluoromethyl, and cyclopropyl.

15. The cyclic 2-aminopyrimidine compound or its pharmaceutically acceptable salt or stereoisomer or prodrug molecule according to claim 1, wherein R₄ is selected from H, methyl; R₂ is selected from: chlorine, bromine, fluorine, iodine, methyl, methoxy, methoxycarbonyl, ethoxycarbonyl, isopropoxycarbonyl, nitro, amino, trifluoromethyl, acetamido-, acrylamido-, isobutyramido-, propionamido-, cyano; R₃ is H.

16. The cyclic 2-aminopyrimidine compound or its pharmaceutically acceptable salt or stereoisomer or prodrug molecule according to claim 1, wherein R₄ is selected from: methoxy, ethoxy, isopropoxy, n-propoxy, isobutyloxy, deuterated methoxy, trifluoromethoxy, cyclopropoxy, trifluoromethyl, cyclopropyl, isopropyl, and ethyl; R₂ is selected from: H, chlorine, bromine, fluorine, iodine, methyl, methoxy, methoxycarbonyl, ethoxycarbonyl, isopropoxycarbonyl, nitro, amino, trifluoromethyl, acetamido-, acrylamido-, isobutyramido-, propionamido-, cyano; R₃ is H.

17. The cyclic 2-aminopyrimidine compound or its pharmaceutically acceptable salt or stereoisomer or prodrug molecule according to claim 1, wherein R₁ is selected from: ethanesulfonyl, methylsulfonyl, and propanesulfonyl; one of R₂ and R₄ is H, and the other is not H; R₃ is H.

18. The cyclic 2-aminopyrimidine compound or its pharmaceutically acceptable salt or stereoisomer or prodrug molecule according to claim 1, wherein R₆ is selected from: —NR₁₃R₁₄, —CR₁₃R₁₄R₁₅, —(CH₂)ₙR₁₃R₁₅, —(CH₂)ₙNR₁₃R₁₄; each R₁₃ and R₁₄ is independently selected from H, C₁˜C₄ alkyl, dimethylamino substituted C₁˜C₄ alkyl, or R₁₃, R₁₄ together with the attached N or C form R₁₇ substituted or unsubstituted 3-12-membered heterocyclic groups; R₁₅ is selected from: H, C₁˜C₄ alkyl;
each R₁₇ is independently selected from: H, deuterium, C₁˜C₈ alkyl, hydroxyl substituted C₁˜C₈ alkyl, C₃˜C₈ cycloalkyl, C₁˜C₈ alkoxy, C₁˜C₈ alkylthio, C₁˜C₈ alkylamino, halogen, hydroxyl, cyano, amino, R₁₈ substituted or unsubstituted 3-8-membered heterocyclic group, —C(═O)R₁₁, —S(═O)₂R₁₁, —S(═O)R₁₁, or R₁₇ together with the attached carbon atom form C(═O); R₁₈ is selected from: H, C₁˜C₈ alkyl.

19. The cyclic 2-aminopyrimidine compound or its pharmaceutically acceptable salt or stereoisomer or prodrug molecule according to claim 18, wherein each R₁₇ is independently selected from: H, deuterium, C₁˜C₄ alkyl, hydroxyl substituted C₁˜C₄ alkyl, C₅˜C₆ cycloalkyl, C₁˜C₄ alkoxy, C₁˜C₄ alkylthio, dimethylamino, halogen, hydroxyl, cyano, amino, R₁₈ substituted or unsubstituted 3-8-membered heterocyclic group, —C(═O)R₁₁, —S(═O)₂R₁₁, —S(═O)R₁₁, or R₁₇ together with the attached carbon atom form C(═O); R₁₈ is selected from: H, C₁˜C₄ alkyl;
each R₁₁ in R₁₇ is independently selected from: C₁˜C₄ alkyl, C₁˜C₄ alkoxy.

20. The cyclic 2-aminopyrimidine compound or its pharmaceutically acceptable salt or stereoisomer or prodrug molecule according to claim 19, wherein R₆ is selected from:

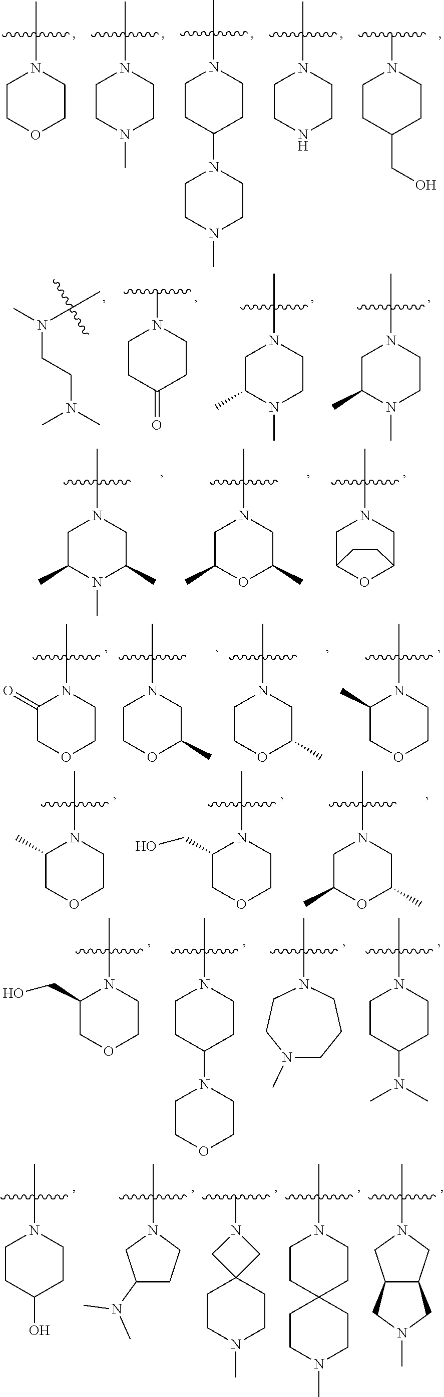

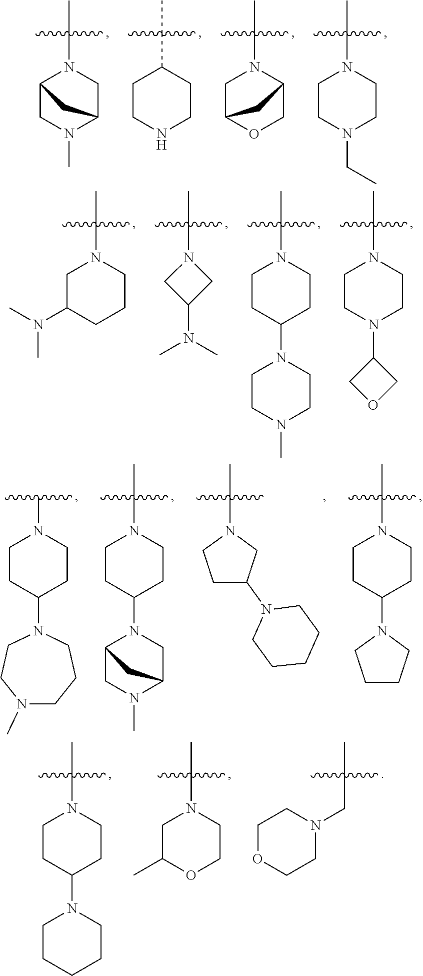

21. A cyclic 2-aminopyrimidine compound or its pharmaceutically acceptable salt or stereoisomer or prodrug molecule according to claim 12, wherein R is selected from H, methoxycarbonyl, isopropoxycarbonyl, aminoformyl, methyl, ethoxycarbonyl, or R together with the attached carbon atom form C(═O).

22. The cyclic 2-aminopyrimidine compound or its pharmaceutically acceptable salt or stereoisomer or prodrug molecule according to claim 1, wherein L is selected from R₁₆ substituted or unsubstituted C₂˜C₆ alkylene, R₁₆ substituted or unsubstituted C₂˜C₆ alkenediyl;
R₁₆ is selected from: H, deuterium, C₁˜C₄ alkyl, halogen substituted C₁˜C₄ alkyl, amino, halogen, hydroxyl, cyano, C₁˜C₄ alkoxy, C₁˜C₄ alkylthio, C₁˜C₄ alkylamino; alternatively, a cyclopropyl group formed by two R₁₆ groups connected to the same carbon atom.

23. The cyclic 2-aminopyrimidine compound or its pharmaceutically acceptable salt or stereoisomer or prodrug molecule according to claim 22, wherein L is selected from: —(CH₂)₂—, —(CH₂)₃—, —(CH₂)₄—,

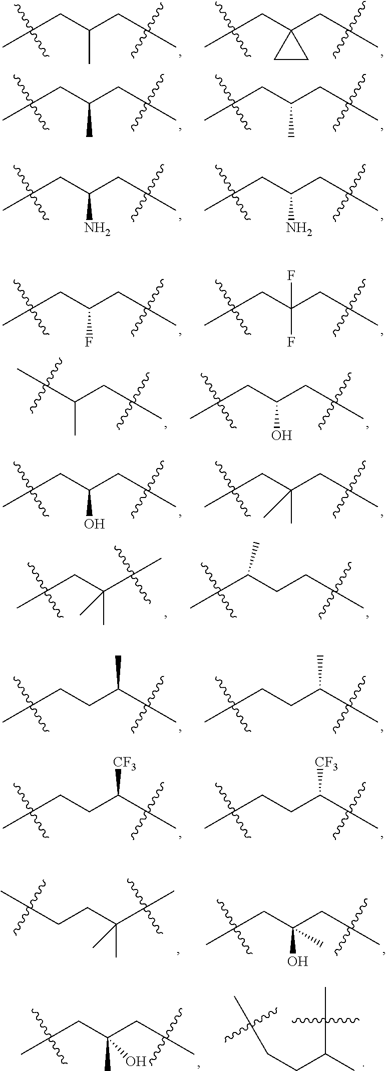

24. The cyclic 2-aminopyrimidine compound or its pharmaceutically acceptable salt or stereoisomer or prodrug molecule according to claim 1, being selected from the following compounds:

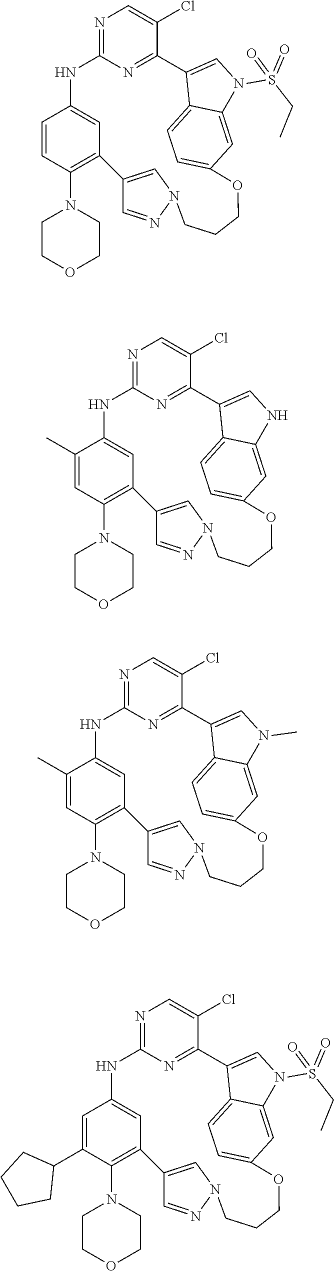

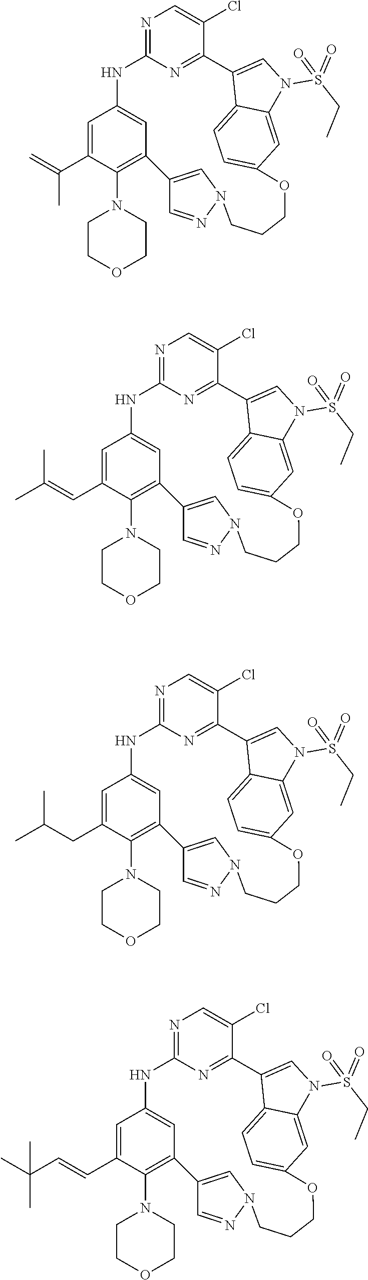 [SMILES: C=C(C)c1cc2cc(c1N1CCOCC1)-c1cnn(c1)CCCOc1ccc3c(cn(S(=O)(=O)CC)c3c1)-c1nc(ncc1Cl)N2 ; CCS(=O)(=O)n1cc2c3ccc(cc31)OCCCn1cc(cn1)-c1cc(cc(C=C(C)C)c1N1CCOCC1)Nc1ncc(Cl)c-2n1 ; CCS(=O)(=O)n1cc2c3ccc(cc31)OCCCn1cc(cn1)-c1cc(cc(CC(C)C)c1N1CCOCC1)Nc1ncc(Cl)c-2n1 ; CCS(=O)(=O)n1cc2c3ccc(cc31)OCCCn1cc(cn1)-c1cc(cc(/C=C/C(C)(C)C)c1N1CCOCC1)Nc1ncc(Cl)c-2n1]

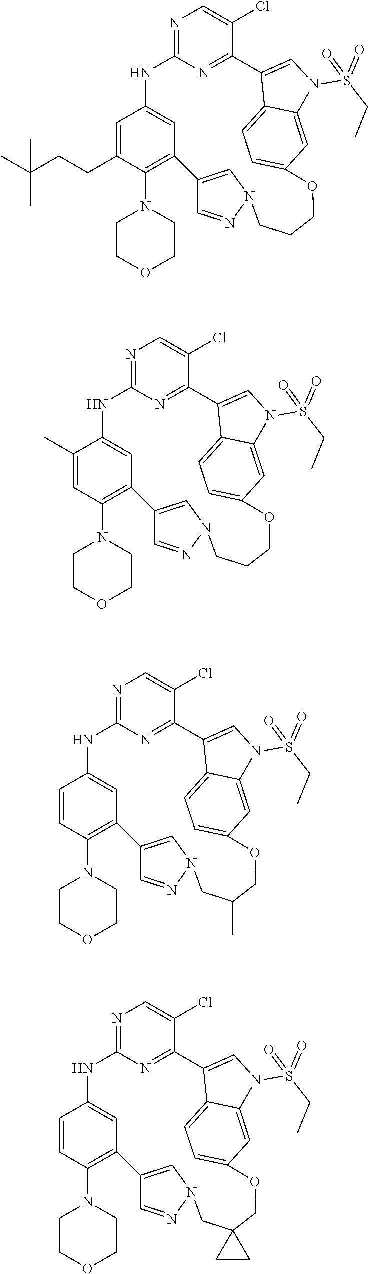 [SMILES: CCS(=O)(=O)n1cc2c3ccc(cc31)OCCCn1cc(cn1)-c1cc(cc(CCC(C)(C)C)c1N1CCOCC1)Nc1ncc(Cl)c-2n1 ; CCS(=O)(=O)n1cc2c3ccc(cc31)OCCCn1cc(cn1)-c1cc(c(C)cc1N1CCOCC1)Nc1ncc(Cl)c-2n1 ; CCS(=O)(=O)n1cc2c3ccc(cc31)OCC(C)Cn1cc(cn1)-c1cc(ccc1N1CCOCC1)Nc1ncc(Cl)c-2n1 ; CCS(=O)(=O)n1cc2c3ccc(cc31)OCC1(CC1)Cn1cc(cn1)-c1cc(ccc1N1CCOCC1)Nc1ncc(Cl)c-2n1]

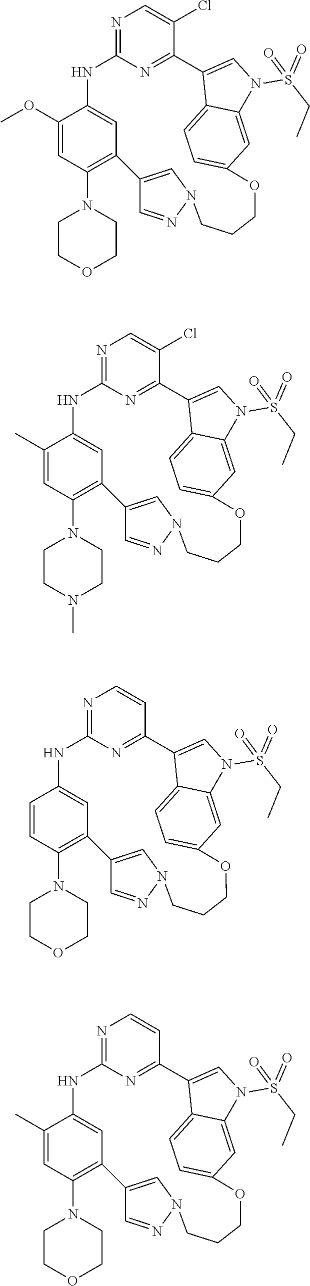 [SMILES: CCS(=O)(=O)n1cc2c3ccc(cc31)OCCCn1cc(cn1)-c1cc(c(OC)cc1N1CCOCC1)Nc1ncc(Cl)c-2n1 ; CCS(=O)(=O)n1cc2c3ccc(cc31)OCCCn1cc(cn1)-c1cc(c(C)cc1N1CCN(C)CC1)Nc1ncc(Cl)c-2n1 ; CCS(=O)(=O)n1cc2c3ccc(cc31)OCCCn1cc(cn1)-c1cc(ccc1N1CCOCC1)Nc1nccc-2n1 ; CCS(=O)(=O)n1cc2c3ccc(cc31)OCCCn1cc(cn1)-c1cc(c(C)cc1N1CCOCC1)Nc1nccc-2n1]

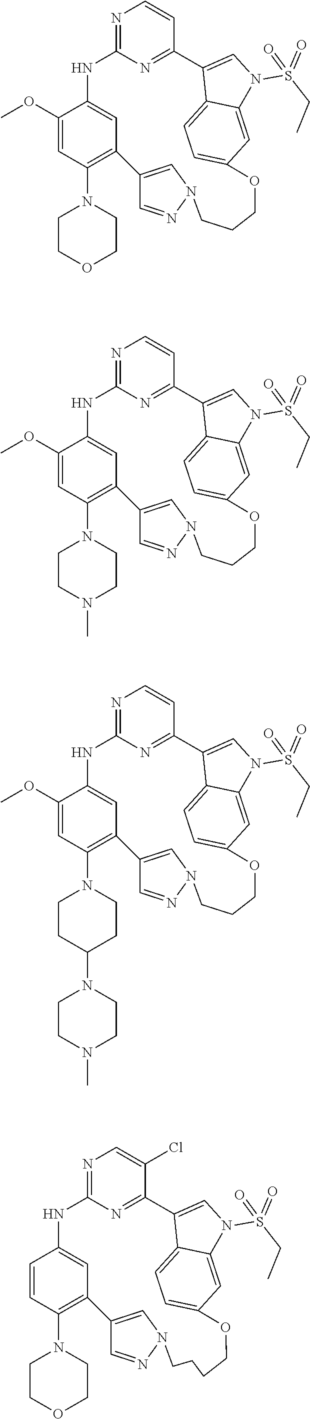 [SMILES: CCS(=O)(=O)n1cc2c3ccc(cc31)OCCCn1cc(cn1)-c1cc(c(OC)cc1N1CCOCC1)Nc1nccc-2n1 ; CCS(=O)(=O)n1cc2c3ccc(cc31)OCCCn1cc(cn1)-c1cc(c(OC)cc1N1CCN(C)CC1)Nc1nccc-2n1 ; CCS(=O)(=O)n1cc2c3ccc(cc31)OCCCn1cc(cn1)-c1cc(c(OC)cc1N1CCC(N3CCN(C)CC3)CC1)Nc1nccc-2n1 ; CCS(=O)(=O)n1cc2c3ccc(cc31)OCCCCn1cc(cn1)-c1cc(ccc1N1CCOCC1)Nc1ncc(Cl)c-2n1]

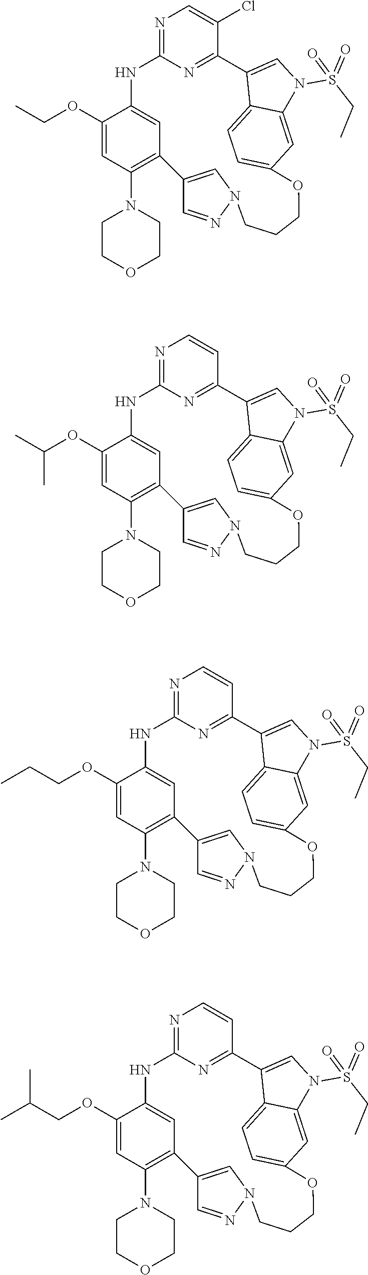 [SMILES: CCOc1cc(N2CCOCC2)c2cc1Nc1ncc(Cl)c(n1)-c1cn(S(=O)(=O)CC)c3cc(ccc13)OCCCn1cc-2cn1 ; CCS(=O)(=O)n1cc2c3ccc(cc31)OCCCn1cc(cn1)-c1cc(c(OC(C)C)cc1N1CCOCC1)Nc1nccc-2n1 ; CCCOc1cc(N2CCOCC2)c2cc1Nc1nccc(n1)-c1cn(S(=O)(=O)CC)c3cc(ccc13)OCCCn1cc-2cn1 ; CCS(=O)(=O)n1cc2c3ccc(cc31)OCCCn1cc(cn1)-c1cc(c(OCC(C)C)cc1N1CCOCC1)Nc1nccc-2n1]

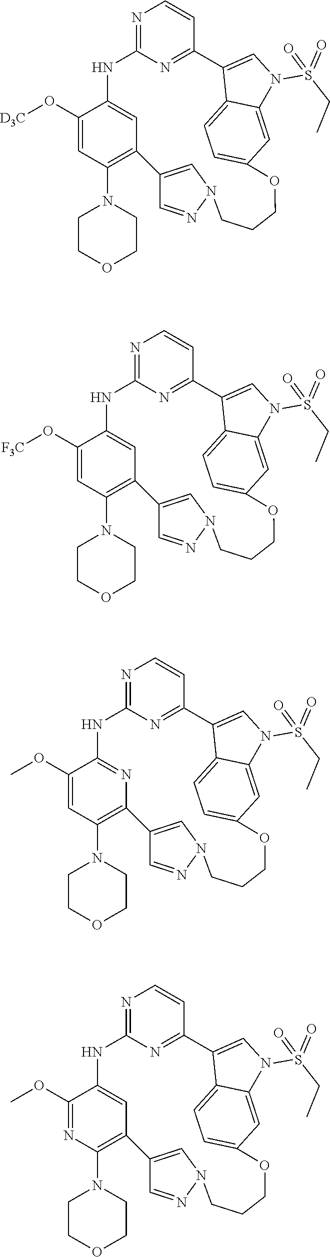 [SMILES: [2H]C([2H])([2H])Oc1cc(N2CCOCC2)c2cc1Nc1nccc(n1)-c1cn(S(=O)(=O)CC)c3cc(ccc13)OCCCn1cc-2cn1 ; CCS(=O)(=O)n1cc2c3ccc(cc31)OCCCn1cc(cn1)-c1cc(c(OC(F)(F)F)cc1N1CCOCC1)Nc1nccc-2n1 ; CCS(=O)(=O)n1cc2c3ccc(cc31)OCCCn1cc(cn1)-c1nc(c(OC)cc1N1CCOCC1)Nc1nccc-2n1 ; CCS(=O)(=O)n1cc2c3ccc(cc31)OCCCn1cc(cn1)-c1cc(c(OC)nc1N1CCOCC1)Nc1nccc-2n1]

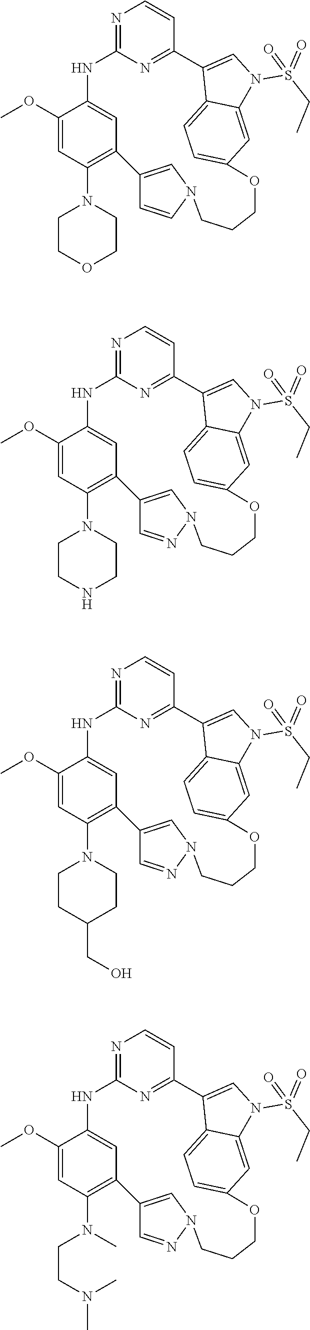 [SMILES: CCS(=O)(=O)n1cc2c3ccc(cc31)OCCCn1ccc(c1)-c1cc(c(OC)cc1N1CCOCC1)Nc1nccc-2n1 ; CCS(=O)(=O)n1cc2c3ccc(cc31)OCCCn1cc(cn1)-c1cc(c(OC)cc1N1CCNCC1)Nc1nccc-2n1 ; CCS(=O)(=O)n1cc2c3ccc(cc31)OCCCn1cc(cn1)-c1cc(c(OC)cc1N1CCC(CO)CC1)Nc1nccc-2n1 ; CCS(=O)(=O)n1cc2c3ccc(cc31)OCCCn1cc(cn1)-c1cc(c(OC)cc1N(C)CCN(C)C)Nc1nccc-2n1]

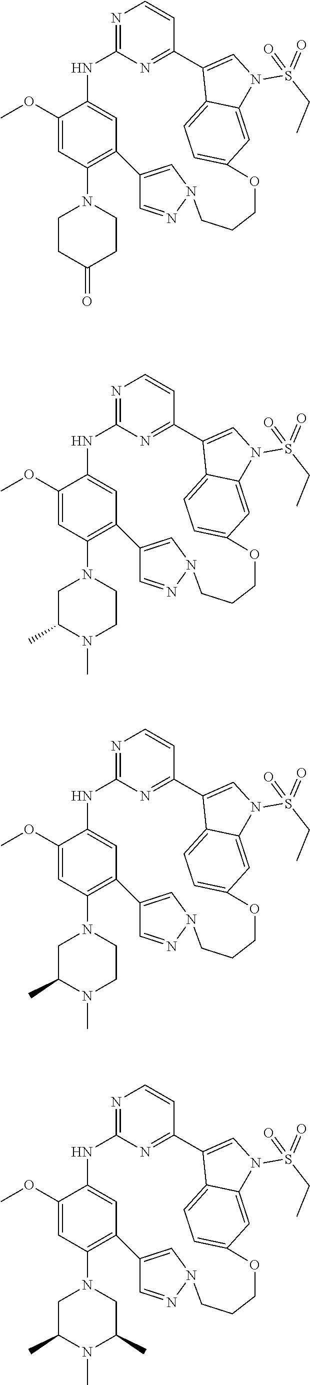 [SMILES: CCS(=O)(=O)n1cc2c3ccc(cc31)OCCCn1cc(cn1)-c1cc(c(OC)cc1N1CCC(=O)CC1)Nc1nccc-2n1 ; CCS(=O)(=O)n1cc2c3ccc(cc31)OCCCn1cc(cn1)-c1cc(c(OC)cc1N1CCN(C)[C@H](C)C1)Nc1nccc-2n1 ; CCS(=O)(=O)n1cc2c3ccc(cc31)OCCCn1cc(cn1)-c1cc(c(OC)cc1N1CCN(C)[C@@H](C)C1)Nc1nccc-2n1 ; CCS(=O)(=O)n1cc2c3ccc(cc31)OCCCn1cc(cn1)-c1cc(c(OC)cc1N1C[C@@H](C)N(C)[C@@H](C)C1)Nc1nccc-2n1]

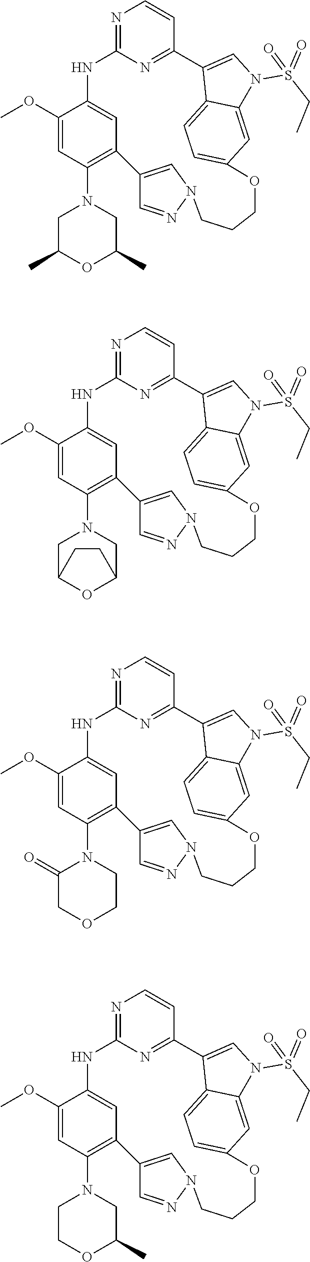 [SMILES: CCS(=O)(=O)n1cc2c3ccc(cc31)OCCCn1cc(cn1)-c1cc(c(OC)cc1N1C[C@@H](C)O[C@@H](C)C1)Nc1nccc-2n1 ; CCS(=O)(=O)n1cc2c3ccc(cc31)OCCCn1cc(cn1)-c1cc(c(OC)cc1N1CC3CCC(C1)O3)Nc1nccc-2n1 ; CCS(=O)(=O)n1cc2c3ccc(cc31)OCCCn1cc(cn1)-c1cc(c(OC)cc1N1CCOCC1=O)Nc1nccc-2n1 ; CCS(=O)(=O)n1cc2c3ccc(cc31)OCCCn1cc(cn1)-c1cc(c(OC)cc1N1CCO[C@H](C)C1)Nc1nccc-2n1]

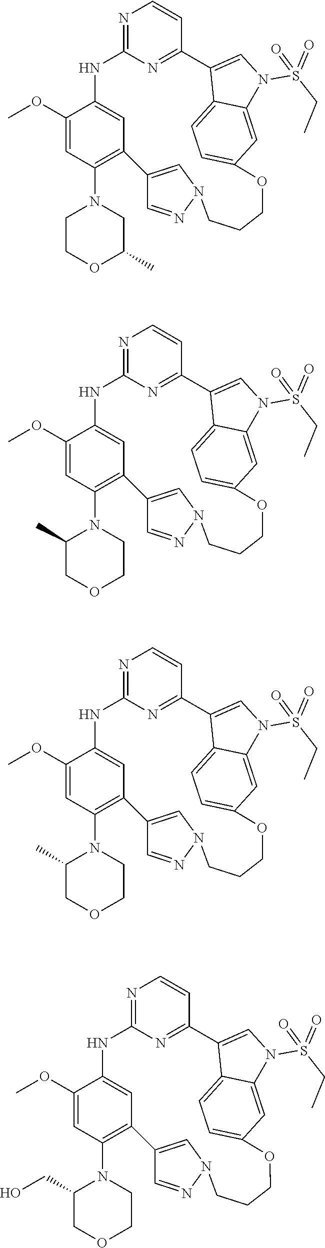 [SMILES: CCS(=O)(=O)n1cc2c3ccc(cc31)OCCCn1cc(cn1)-c1cc(c(OC)cc1N1CCO[C@@H](C)C1)Nc1nccc-2n1 ; CCS(=O)(=O)n1cc2c3ccc(cc31)OCCCn1cc(cn1)-c1cc(c(OC)cc1N1CCOC[C@H]1C)Nc1nccc-2n1 ; CCS(=O)(=O)n1cc2c3ccc(cc31)OCCCn1cc(cn1)-c1cc(c(OC)cc1N1CCOC[C@@H]1C)Nc1nccc-2n1 ; CCS(=O)(=O)n1cc2c3ccc(cc31)OCCCn1cc(cn1)-c1cc(c(OC)cc1N1CCOC[C@@H]1CO)Nc1nccc-2n1]

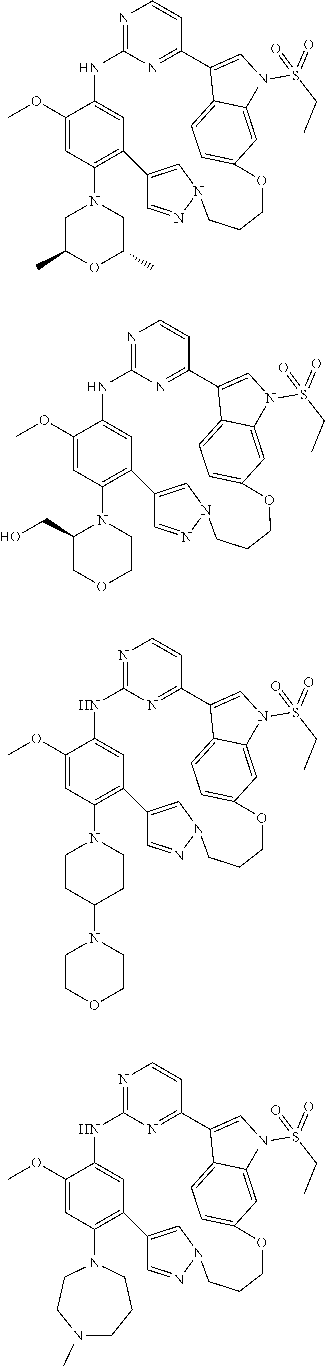 [SMILES: CCS(=O)(=O)n1cc2c3ccc(cc31)OCCCn1cc(cn1)-c1cc(c(OC)cc1N1C[C@H](C)O[C@@H](C)C1)Nc1nccc-2n1 ; CCS(=O)(=O)n1cc2c3ccc(cc31)OCCCn1cc(cn1)-c1cc(c(OC)cc1N1CCOC[C@H]1CO)Nc1nccc-2n1 ; CCS(=O)(=O)n1cc2c3ccc(cc31)OCCCn1cc(cn1)-c1cc(c(OC)cc1N1CCC(N3CCOCC3)CC1)Nc1nccc-2n1 ; CCS(=O)(=O)n1cc2c3ccc(cc31)OCCCn1cc(cn1)-c1cc(c(OC)cc1N1CCCN(C)CC1)Nc1nccc-2n1]

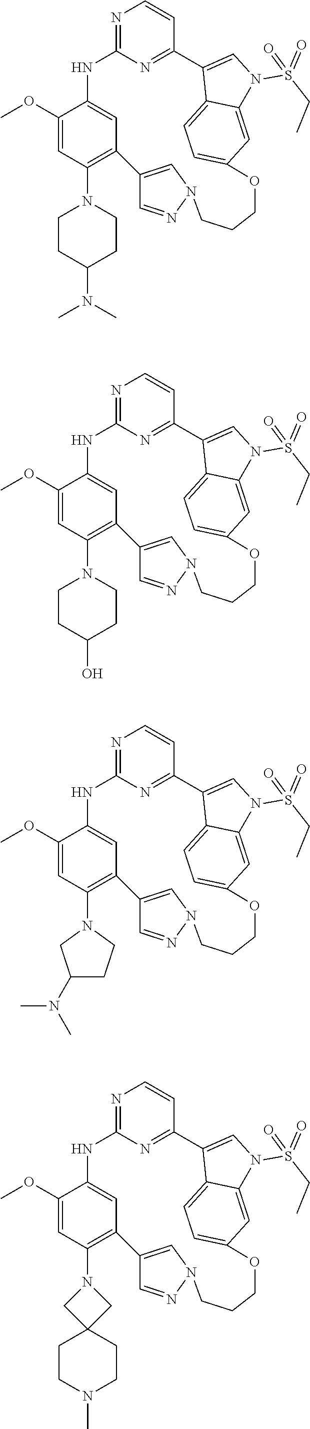 [SMILES: CCS(=O)(=O)n1cc2c3ccc(cc31)OCCCn1cc(cn1)-c1cc(c(OC)cc1N1CCC(N(C)C)CC1)Nc1nccc-2n1 ; CCS(=O)(=O)n1cc2c3ccc(cc31)OCCCn1cc(cn1)-c1cc(c(OC)cc1N1CCC(O)CC1)Nc1nccc-2n1 ; CCS(=O)(=O)n1cc2c3ccc(cc31)OCCCn1cc(cn1)-c1cc(c(OC)cc1N1CCC(N(C)C)C1)Nc1nccc-2n1 ; CCS(=O)(=O)n1cc2c3ccc(cc31)OCCCn1cc(cn1)-c1cc(c(OC)cc1N1CC3(CCN(C)CC3)C1)Nc1nccc-2n1]

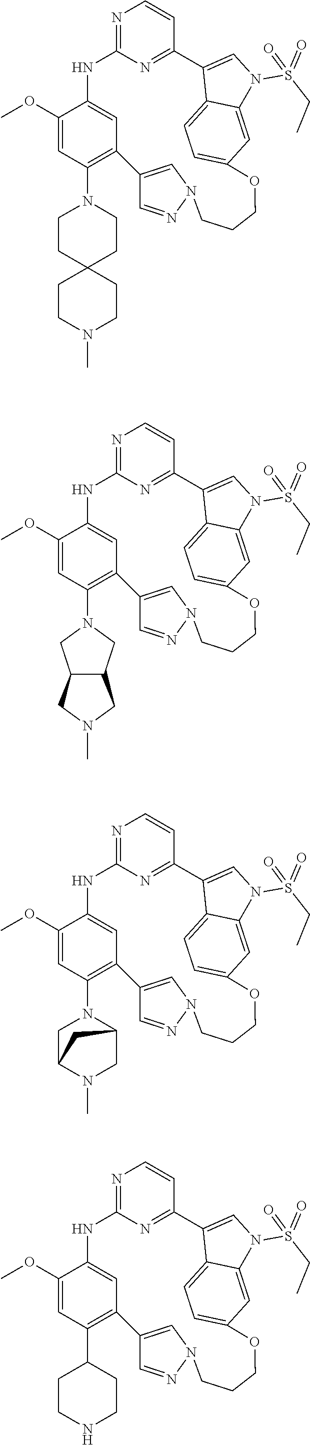

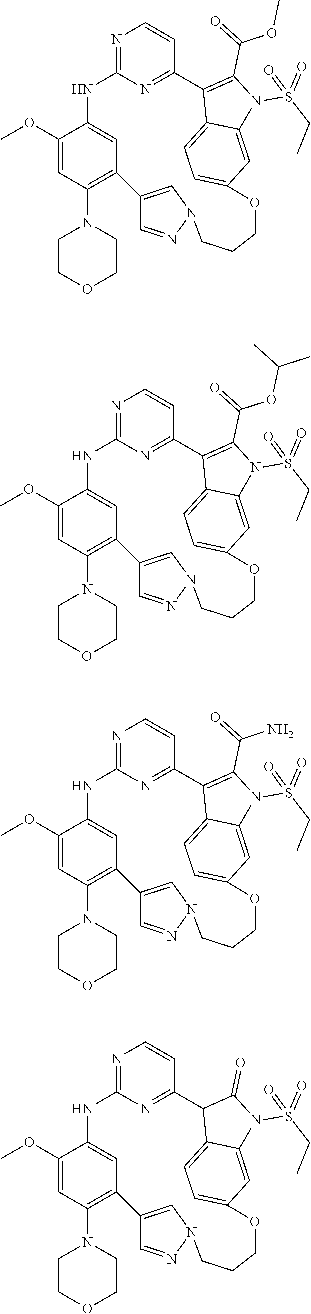

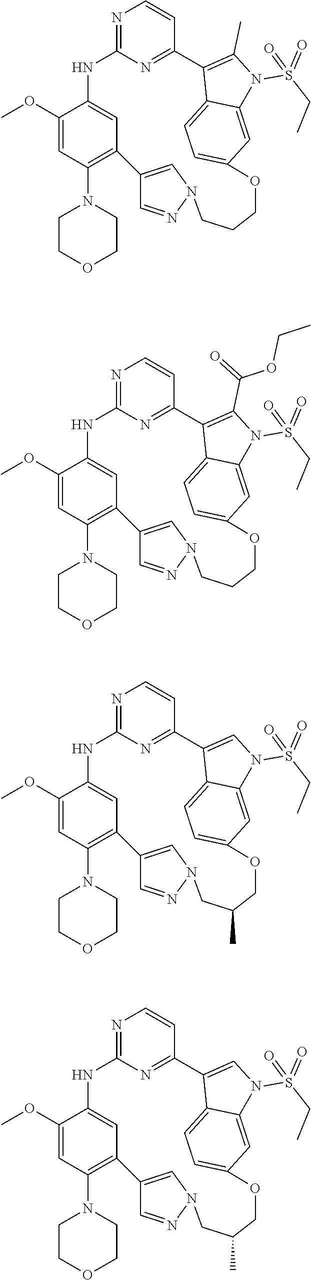

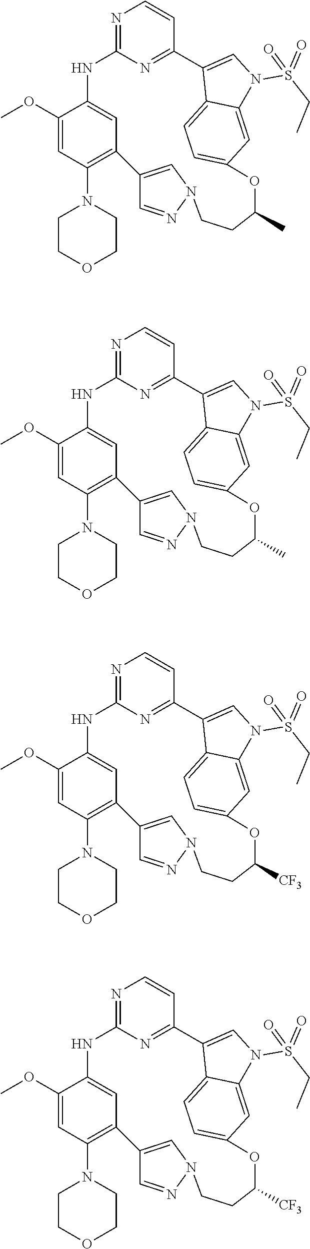 [SMILES: CCS(=O)(=O)n1cc2c3ccc(cc31)O[C@@H](C)CCn1cc(cn1)-c1cc(c(OC)cc1N1CCOCC1)Nc1nccc-2n1 ; CCS(=O)(=O)n1cc2c3ccc(cc31)O[C@H](C)CCn1cc(cn1)-c1cc(c(OC)cc1N1CCOCC1)Nc1nccc-2n1 ; CCS(=O)(=O)n1cc2c3ccc(cc31)O[C@@H](C(F)(F)F)CCn1cc(cn1)-c1cc(c(OC)cc1N1CCOCC1)Nc1nccc-2n1 ; CCS(=O)(=O)n1cc2c3ccc(cc31)O[C@H](C(F)(F)F)CCn1cc(cn1)-c1cc(c(OC)cc1N1CCOCC1)Nc1nccc-2n1]

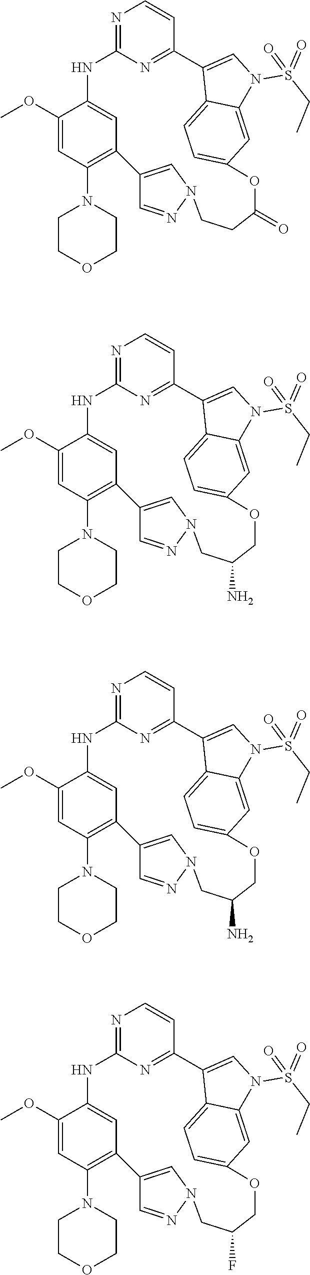 [SMILES: CCS(=O)(=O)n1cc2c3ccc(cc31)OC(=O)CCn1cc(cn1)-c1cc(c(OC)cc1N1CCOCC1)Nc1nccc-2n1 ; CCS(=O)(=O)n1cc2c3ccc(cc31)OC[C@H](N)Cn1cc(cn1)-c1cc(c(OC)cc1N1CCOCC1)Nc1nccc-2n1 ; CCS(=O)(=O)n1cc2c3ccc(cc31)OC[C@@H](N)Cn1cc(cn1)-c1cc(c(OC)cc1N1CCOCC1)Nc1nccc-2n1 ; CCS(=O)(=O)n1cc2c3ccc(cc31)OC[C@H](F)Cn1cc(cn1)-c1cc(c(OC)cc1N1CCOCC1)Nc1nccc-2n1]

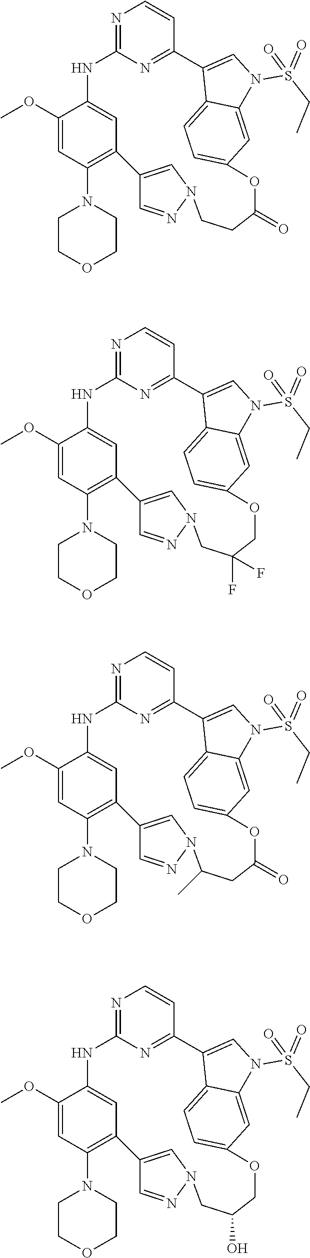 [SMILES: CCS(=O)(=O)n1cc2c3ccc(cc31)OC(=O)CCn1cc(cn1)-c1cc(c(OC)cc1N1CCOCC1)Nc1nccc-2n1 ; CCS(=O)(=O)n1cc2c3ccc(cc31)OCC(F)(F)Cn1cc(cn1)-c1cc(c(OC)cc1N1CCOCC1)Nc1nccc-2n1 ; CCS(=O)(=O)n1cc2c3ccc(cc31)OC(=O)CC(C)n1cc(cn1)-c1cc(c(OC)cc1N1CCOCC1)Nc1nccc-2n1 ; CCS(=O)(=O)n1cc2c3ccc(cc31)OC[C@H](O)Cn1cc(cn1)-c1cc(c(OC)cc1N1CCOCC1)Nc1nccc-2n1]

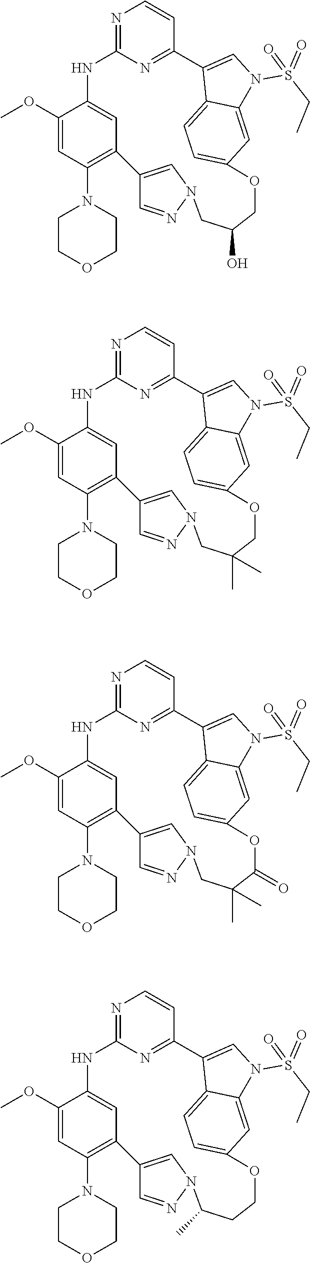 [SMILES: CCS(=O)(=O)n1cc2c3ccc(cc31)OC[C@@H](O)Cn1cc(cn1)-c1cc(c(OC)cc1N1CCOCC1)Nc1nccc-2n1 ; CCS(=O)(=O)n1cc2c3ccc(cc31)OCC(C)(C)Cn1cc(cn1)-c1cc(c(OC)cc1N1CCOCC1)Nc1nccc-2n1 ; CCS(=O)(=O)n1cc2c3ccc(cc31)OC(=O)C(C)(C)Cn1cc(cn1)-c1cc(c(OC)cc1N1CCOCC1)Nc1nccc-2n1 ; CCS(=O)(=O)n1cc2c3ccc(cc31)OCC[C@H](C)n1cc(cn1)-c1cc(c(OC)cc1N1CCOCC1)Nc1nccc-2n1]

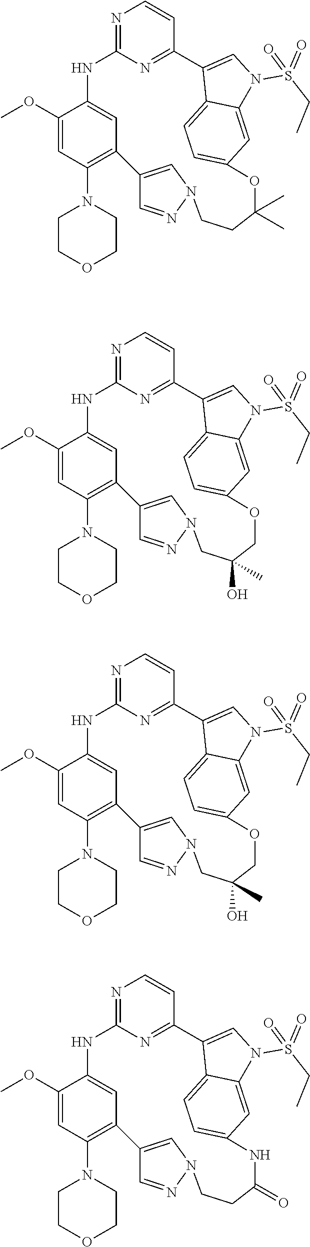 [SMILES: CCS(=O)(=O)n1cc2c3ccc(cc31)OC(C)(C)CCn1cc(cn1)-c1cc(c(OC)cc1N1CCOCC1)Nc1nccc-2n1 ; CCS(=O)(=O)n1cc2c3ccc(cc31)OC[C@@](C)(O)Cn1cc(cn1)-c1cc(c(OC)cc1N1CCOCC1)Nc1nccc-2n1 ; CCS(=O)(=O)n1cc2c3ccc(cc31)OC[C@](C)(O)Cn1cc(cn1)-c1cc(c(OC)cc1N1CCOCC1)Nc1nccc-2n1 ; CCS(=O)(=O)n1cc2c3ccc(cc31)NC(=O)CCn1cc(cn1)-c1cc(c(OC)cc1N1CCOCC1)Nc1nccc-2n1]

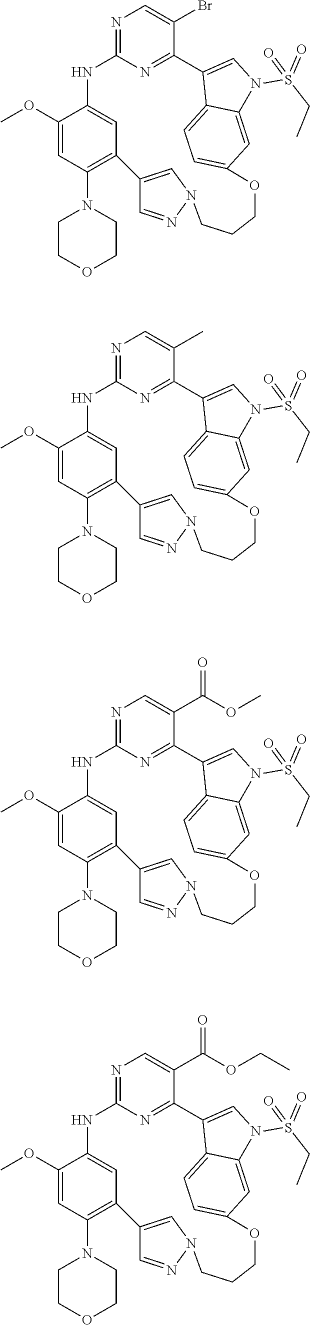 [SMILES: CCS(=O)(=O)n1cc2c3ccc(cc31)OCCCn1cc(cn1)-c1cc(c(OC)cc1N1CCOCC1)Nc1ncc(Br)c-2n1 ; CCS(=O)(=O)n1cc2c3ccc(cc31)OCCCn1cc(cn1)-c1cc(c(OC)cc1N1CCOCC1)Nc1ncc(C)c-2n1 ; CCS(=O)(=O)n1cc2c3ccc(cc31)OCCCn1cc(cn1)-c1cc(c(OC)cc1N1CCOCC1)Nc1ncc(C(=O)OC)c-2n1 ; CCOC(=O)c1cnc2nc1-c1cn(S(=O)(=O)CC)c3cc(ccc13)OCCCn1cc(cn1)-c1cc(c(OC)cc1N1CCOCC1)N2]

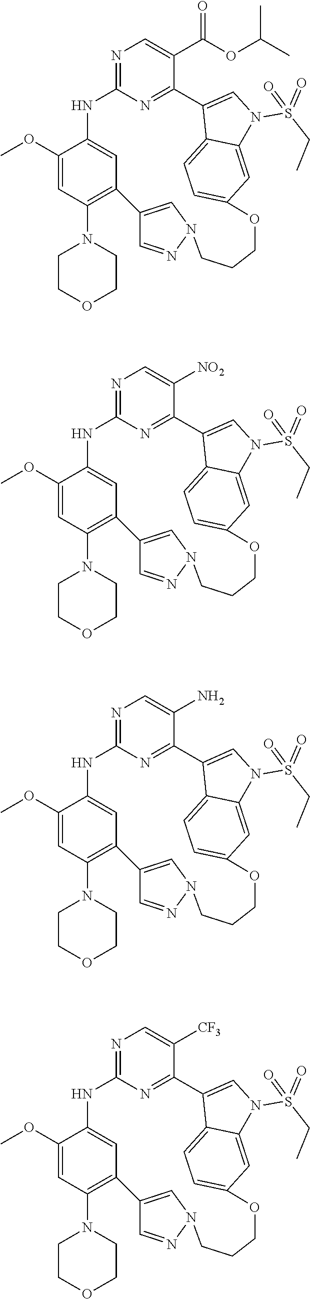 [SMILES: CCS(=O)(=O)n1cc2c3ccc(cc31)OCCCn1cc(cn1)-c1cc(c(OC)cc1N1CCOCC1)Nc1ncc(C(=O)OC(C)C)c-2n1 ; CCS(=O)(=O)n1cc2c3ccc(cc31)OCCCn1cc(cn1)-c1cc(c(OC)cc1N1CCOCC1)Nc1ncc([N+](=O)[O-])c-2n1 ; CCS(=O)(=O)n1cc2c3ccc(cc31)OCCCn1cc(cn1)-c1cc(c(OC)cc1N1CCOCC1)Nc1ncc(N)c-2n1 ; CCS(=O)(=O)n1cc2c3ccc(cc31)OCCCn1cc(cn1)-c1cc(c(OC)cc1N1CCOCC1)Nc1ncc(C(F)(F)F)c-2n1]

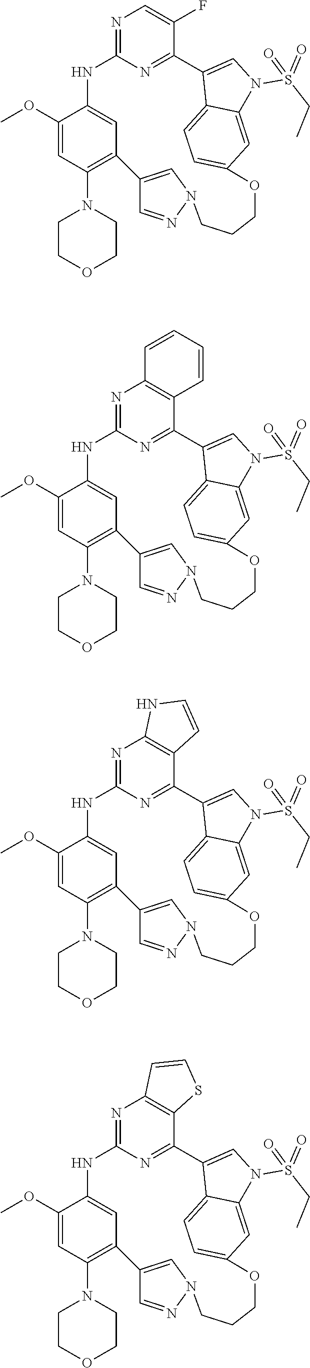 [SMILES: CCS(=O)(=O)n1cc2c3ccc(cc31)OCCCn1cc(cn1)-c1cc(c(OC)cc1N1CCOCC1)Nc1ncc(F)c-2n1 ; CCS(=O)(=O)n1cc2c3ccc(cc31)OCCCn1cc(cn1)-c1cc(c(OC)cc1N1CCOCC1)Nc1nc-2c2ccccc2n1 ; CCS(=O)(=O)n1cc2c3ccc(cc31)OCCCn1cc(cn1)-c1cc(c(OC)cc1N1CCOCC1)Nc1nc-2c2cc[nH]c2n1 ; CCS(=O)(=O)n1cc2c3ccc(cc31)OCCCn1cc(cn1)-c1cc(c(OC)cc1N1CCOCC1)Nc1nc-2c2sccc2n1]

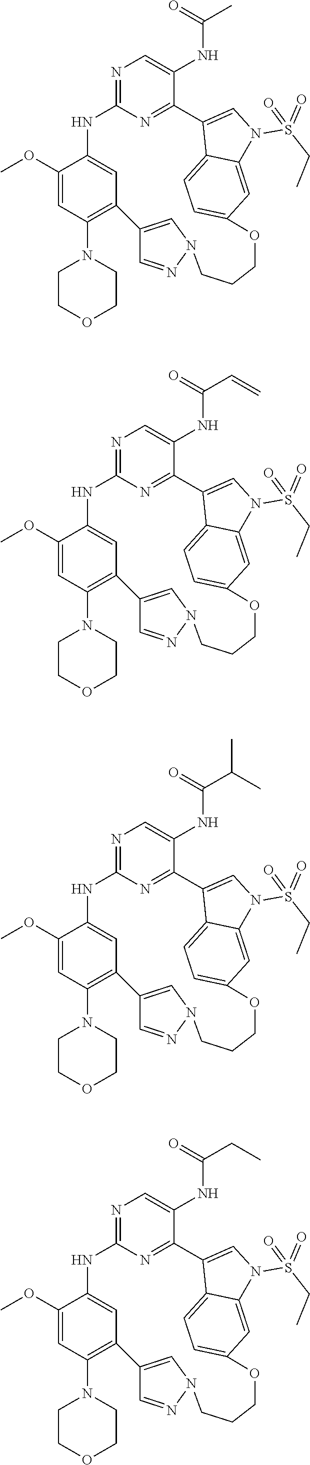 [SMILES: CCS(=O)(=O)n1cc2c3ccc(cc31)OCCCn1cc(cn1)-c1cc(c(OC)cc1N1CCOCC1)Nc1ncc(NC(C)=O)c-2n1 ; C=CC(=O)Nc1cnc2nc1-c1cn(S(=O)(=O)CC)c3cc(ccc13)OCCCn1cc(cn1)-c1cc(c(OC)cc1N1CCOCC1)N2 ; CCS(=O)(=O)n1cc2c3ccc(cc31)OCCCn1cc(cn1)-c1cc(c(OC)cc1N1CCOCC1)Nc1ncc(NC(=O)C(C)C)c-2n1 ; CCC(=O)Nc1cnc2nc1-c1cn(S(=O)(=O)CC)c3cc(ccc13)OCCCn1cc(cn1)-c1cc(c(OC)cc1N1CCOCC1)N2]

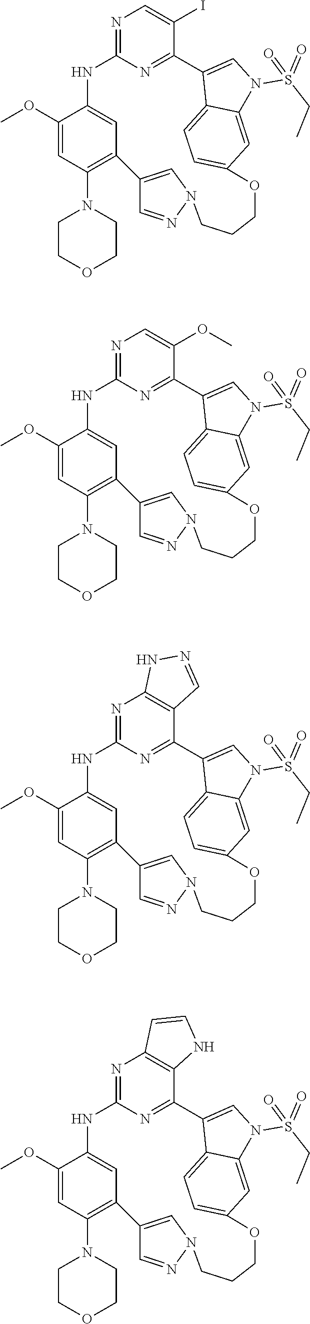 [SMILES: CCS(=O)(=O)n1cc2c3ccc(cc31)OCCCn1cc(cn1)-c1cc(c(OC)cc1N1CCOCC1)Nc1ncc(I)c-2n1 ; CCS(=O)(=O)n1cc2c3ccc(cc31)OCCCn1cc(cn1)-c1cc(c(OC)cc1N1CCOCC1)Nc1ncc(OC)c-2n1 ; CCS(=O)(=O)n1cc2c3ccc(cc31)OCCCn1cc(cn1)-c1cc(c(OC)cc1N1CCOCC1)Nc1nc-2c2cn[nH]c2n1 ; CCS(=O)(=O)n1cc2c3ccc(cc31)OCCCn1cc(cn1)-c1cc(c(OC)cc1N1CCOCC1)Nc1nc-2c2[nH]ccc2n1]

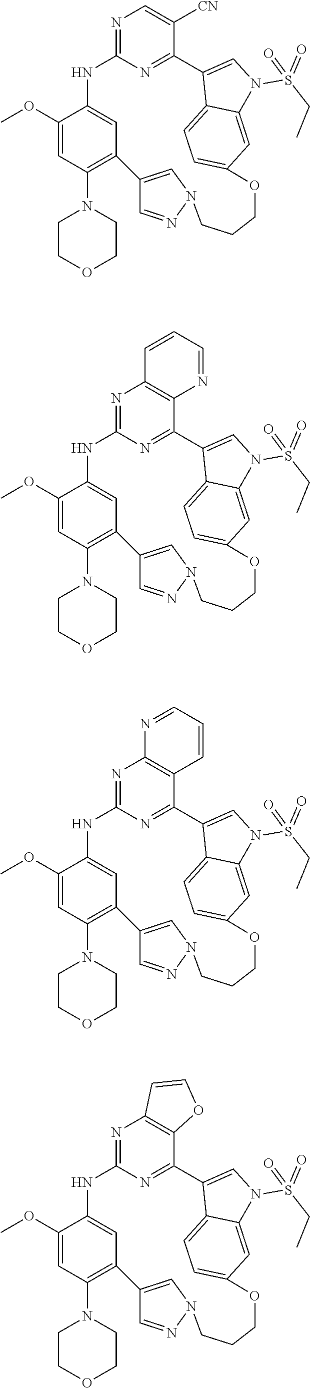 [SMILES: CCS(=O)(=O)n1cc2c3ccc(cc31)OCCCn1cc(cn1)-c1cc(c(OC)cc1N1CCOCC1)Nc1ncc(C#N)c-2n1 ; CCS(=O)(=O)n1cc2c3ccc(cc31)OCCCn1cc(cn1)-c1cc(c(OC)cc1N1CCOCC1)Nc1nc-2c2ncccc2n1 ; CCS(=O)(=O)n1cc2c3ccc(cc31)OCCCn1cc(cn1)-c1cc(c(OC)cc1N1CCOCC1)Nc1nc-2c2cccnc2n1 ; CCS(=O)(=O)n1cc2c3ccc(cc31)OCCCn1cc(cn1)-c1cc(c(OC)cc1N1CCOCC1)Nc1nc-2c2occc2n1]

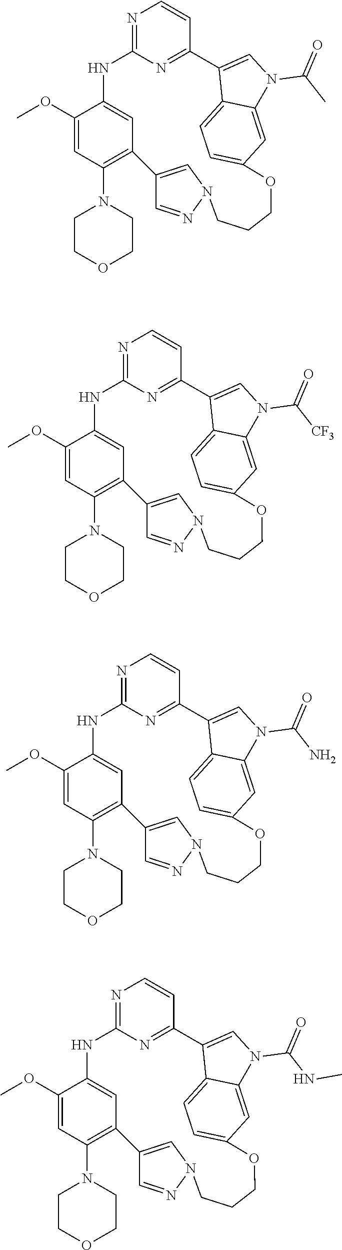

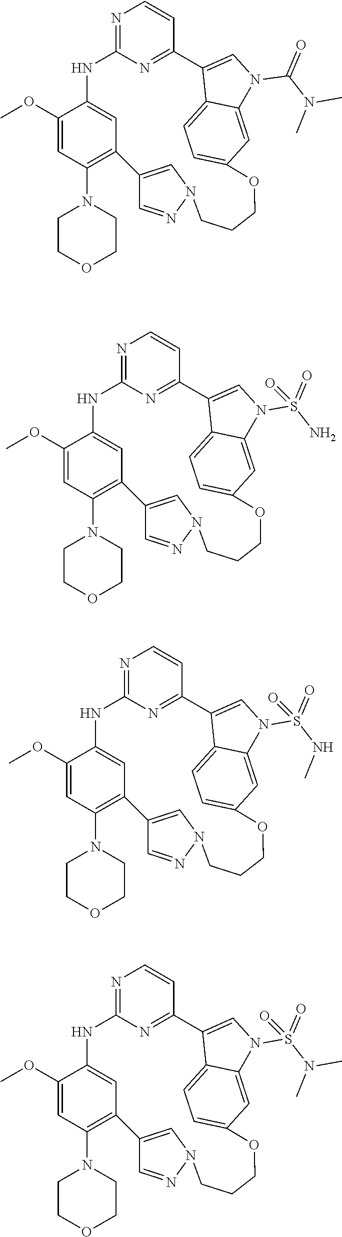

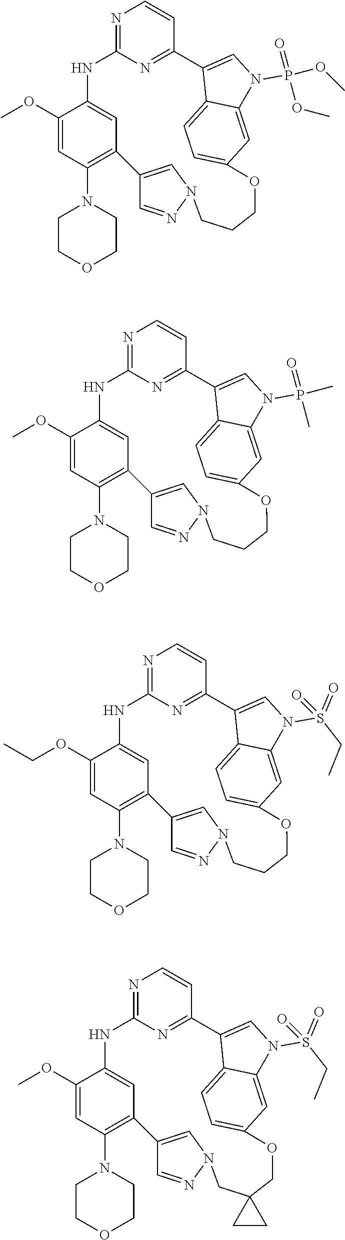

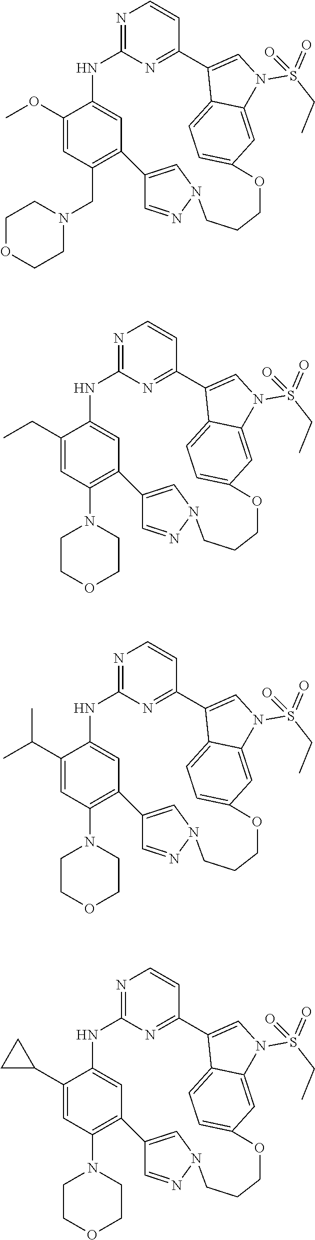 [SMILES: CCS(=O)(=O)n1cc2c3ccc(cc31)OCCCn1cc(cn1)-c1cc(c(OC)cc1CN1CCOCC1)Nc1nccc-2n1 ; CCc1cc(N2CCOCC2)c2cc1Nc1nccc(n1)-c1cn(S(=O)(=O)CC)c3cc(ccc13)OCCCn1cc-2cn1 ; CCS(=O)(=O)n1cc2c3ccc(cc31)OCCCn1cc(cn1)-c1cc(c(C(C)C)cc1N1CCOCC1)Nc1nccc-2n1 ; CCS(=O)(=O)n1cc2c3ccc(cc31)OCCCn1cc(cn1)-c1cc(c(C3CC3)cc1N1CCOCC1)Nc1nccc-2n1]

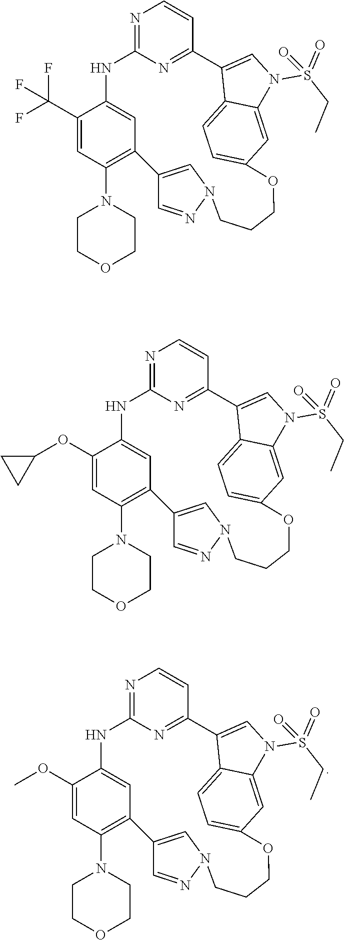 [SMILES: CCS(=O)(=O)n1cc2c3ccc(cc31)OCCCn1cc(cn1)-c1cc(c(C(F)(F)F)cc1N1CCOCC1)Nc1nccc-2n1 ; CCS(=O)(=O)n1cc2c3ccc(cc31)OCCCn1cc(cn1)-c1cc(c(OC3CC3)cc1N1CCOCC1)Nc1nccc-2n1 ; CCS(=O)(=O)n1cc2c3ccc(cc31)OCCCn1cc(cn1)-c1cc(c(OC)cc1N1CCOCC1)Nc1nccc-2n1]

25. The cyclic 2-aminopyrimidine compound or its pharmaceutically acceptable salt or stereoisomer or prodrug molecule according to claim 1, being selected from the following compounds:

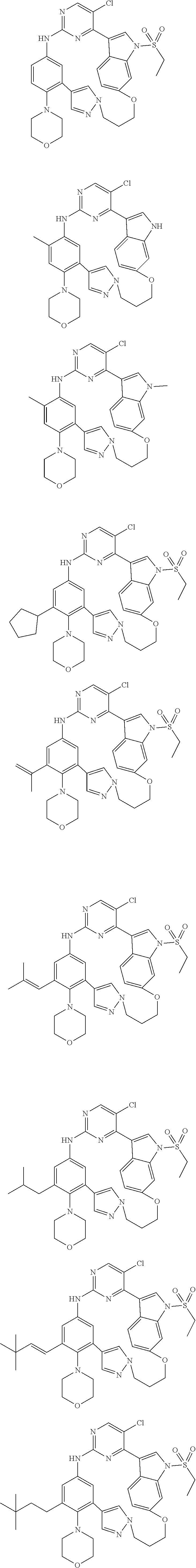

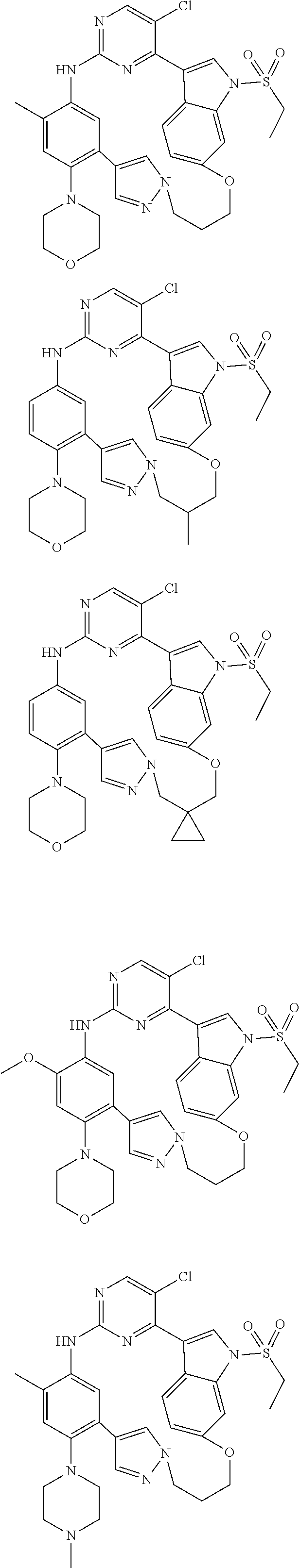

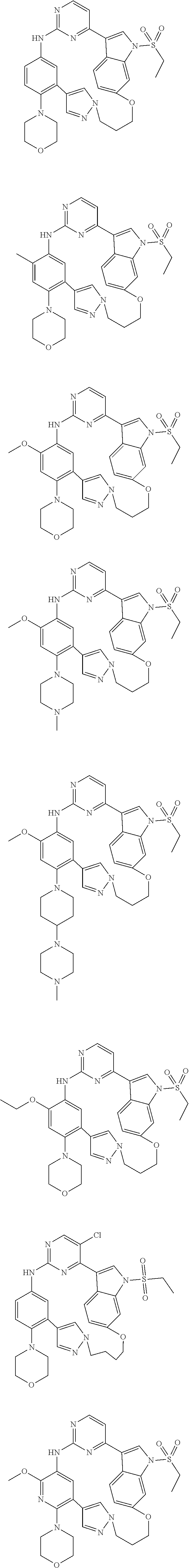

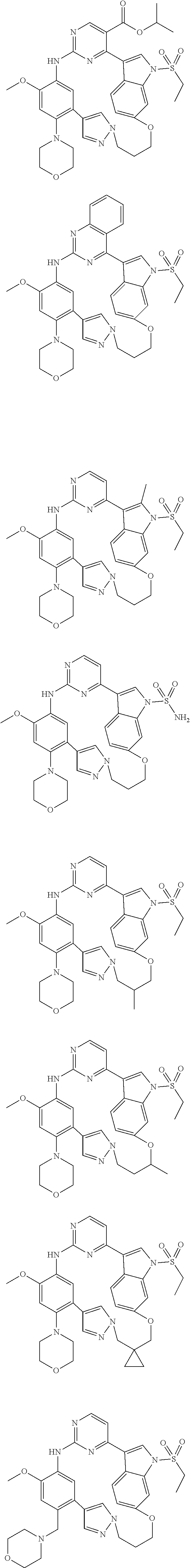

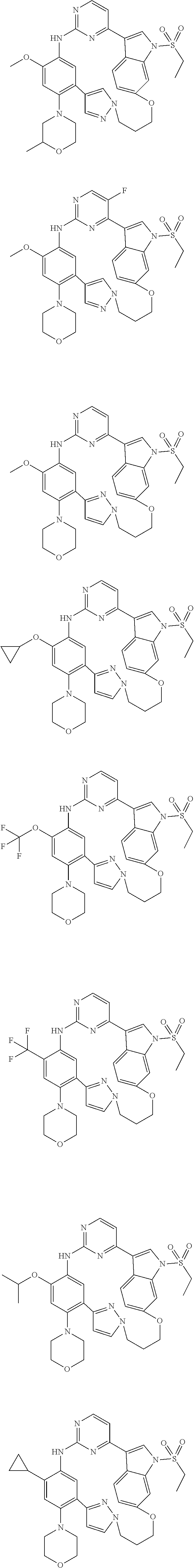

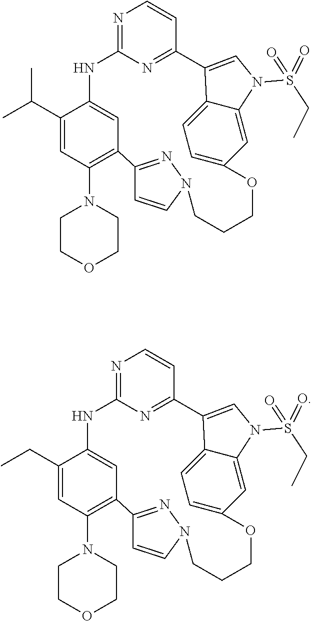 [SMILES: CCS(=O)(=O)n1cc2c3ccc(cc31)OCCCn1ccc(n1)-c1cc(c(C(C)C)cc1N1CCOCC1)Nc1nccc-2n1 ; CCc1cc(N2CCOCC2)c2cc1Nc1nccc(n1)-c1cn(S(=O)(=O)CC)c3cc(ccc13)OCCCn1ccc-2n1]

26. A pharmaceutical composition for preventing and/or treating tumors, being prepared from an active ingredient and a pharmaceutically acceptable excipient, wherein the active ingredient comprises a cyclic 2-aminopyrimidine compound or its pharmaceutically acceptable salt or stereoisomer or a prodrug molecule according to claim 1.

27. A method for preventing and/or treating tumors, comprising: administering to a patient a safe and effective amount of a cyclic 2-aminopyrimidine compound or its pharmaceutically acceptable salt or stereoisomer or prodrug molecule according to claim 1.

28. The method according to claim 27, wherein the tumor is: non-small cell lung cancer, small cell lung cancer, lung adenocarcinoma, lung squamous cell cancer, pancreatic cancer, breast cancer, prostate cancer, liver cancer, skin cancer, epithelial cell cancer, gastrointestinal stromal tumor, leukemia, histiocytic lymphoma, nasopharyngeal cancer, head and neck tumor, colon cancer, rectal cancer or glioma.

29. A method for selectively inhibiting mutant EGFR kinase activity, comprising:
administering to a patient a safe and effective amount of a cyclic 2-aminopyrimidine compound or its pharmaceutically acceptable salt or stereoisomer or prodrug molecule according to claim 1.

30. The method according to claim 29, wherein the mutant EGFR is EGFR^{T790M}, EGFR^{T790M/C797S}, EGFR^{19del/T790M/C797S}, EGFR^{L858R/T790M}, or EGFR^{L858R/C797S}, EGFR^{L858R/T790M/C797S}.
